(19) United States
(12) Patent Application Publication
(10) Pub. No.: US 2009/0226904 A1
(43) Pub. Date: Sep. 10, 2009

(54) DIAGNOSIS AND TREATMENT OF EXOCRINE PANCREATIC DYSFUNCTION AND DIABETES

(75) Inventors: Pål R. Njolstad, Paradis (NO); Helge Raeder, Bergen (NO); Stefan Johansson, Godvik (NO); Pål Ivar Holm, Soereidgrend (NO); Anders Molven, Nesttum (NO)

Correspondence Address: MANELLI DENISON & SELTER, 2000 M STREET NW SUITE 700, WASHINGTON, DC, 20036-3307, US

(73) Assignee: University of Bergen, Bergen (NO)

(21) Appl. No.: 12/095,564

(22) PCT Filed: Dec. 1, 2006
(86) PCT No.: PCT/IB2006/003437

§ 371 Date: Dec. 9, 2008

Related U.S. Application Data
(60) Provisional application No. 60/741,761, filed on Dec. 2, 2005.

(30) Foreign Application Priority Data
Dec. 1, 2005 (DK) PA 2005 01700

(51) Int. Cl. C12Q 1/68

(52) U.S. Cl. 435/6

(57) ABSTRACT

The present invention relates to association of one or more polymorphisms located in the human CEL gene to the occurrence of exocrine pancreatic dysfunction and a endocrine pancreatic dysfunction in the form of diabetes. The invention relates both to methods for diagnosing a predisposition to said diseases, classifying said diseases and to methods and compositions for treating subjects with said diseases. Furthermore the invention relates to screens for identifying compounds effective in treating said diseases. The invention describes specific single nucleotide polymorphisms the presence of which in the genome of an individual is strongly associated with the predisposition of said individual to exocrine pancreatic dysfunction and diabetes.

This application is a non-provisional of U.S. provisional application Ser. No. 60/741,761 filed on Dec. 2, 2005, which is hereby incorporated by reference in its entirety. All patent and non-patent references cited in U.S. provisional application Ser. No. 60/741,761, or in the present application, are incorporated by reference in their entirety.

### FIELD OF INVENTION

The present invention relates to an association of one or more polymorphisms located in the human CEL gene to the occurrence of exocrine pancreatic dysfunction and/or diabetes and to methods and compositions for treating subjects with said diseases. Furthermore the invention relates to screens for identifying compounds effective in treating said diseases.

### BACKGROUND OF INVENTION

### Pancreas

The pancreas serves both endocrine and exocrine functions. The endocrine cells are found in the islets of Langerhans, they synthesize insulin and other hormones, and are involved in the pathogenesis of diabetes mellitus. The exocrine cells produce bicarbonate and digestive enzymes and are involved in the pathogenesis of pancreatic malabsorption. The localization of the islets within exocrine pancreatic tissue is suggestive of an interdependency and cross-talk between these two cell populations in their normal as well as in their abnormal function[1]. Diabetes has been proposed to be both the cause[2] and the consequence of exocrine pancreatic disease[3-5]. In other cases a common factor has been considered to lead to both exocrine pancreas dysfunction and diabetes[6-9], reflecting that exocrine and endocrine pancreatic cells originate from the same pool of endodermal cell[10].

### Polymorphisms

DNA polymorphisms provide an efficient way to study the association of genes and diseases by analysis of linkage and linkage disequilibrium. With the sequencing of the human genome a myriad of hitherto unknown genetic polymorphisms among people have been detected. Most common among these are the single nucleotide polymorphisms, also called SNPs, of which there are known several millions. Other examples are variable number of tandem repeat polymorphisms, insertions, deletions and block modifications. Tandem repeats (VNTR) often have multiple different alleles (variants), whereas the other groups of polymorphisms usually have just two alleles. Some of these genetic polymorphisms probably play a direct role in the biology of the individuals, including their risk of developing disease, but the virtue of the majority is that they can serve as markers for the surrounding DNA. The association of an allele of one sequence polymorphism with particular alleles of other sequence polymorphisms in the surrounding DNA has two origins, known in the genetic field as linkage and linkage disequilibrium, respectively. Linkage arises because large parts of chromosomes are passed unchanged from parents to offspring, so that minor regions of a chromosome tend to flow unchanged from one generation to the next and also to be similar in different branches of the same family. Linkage is gradually eroded by recombination occurring in the cells of the germline, but typically operates over multiple generations and distances of a number of million bases in the DNA.

Linkage disequilibrium deals with whole populations and has its origin in the (distant) forefather in whose DNA a new sequence polymorphism arose. The immediate surroundings in the DNA of the forefather will tend to stay with the new allele for many generations. Recombination and changes in the composition of the population will again erode the association, but the new allele and the alleles of any other polymorphism nearby will often be partly associated among unrelated humans even today. A crude estimate suggests that alleles of sequence polymorphisms with distances less than 10000 bases in the DNA will have tended to stay together since modern man arose. Linkage disequilbrium in limited populations, for instance Europeans, often extends over longer distances, e.g. over more than 1,000,000 bases. This can be the result of newer mutations, but can also be a consequence of one or more “bottlenecks” with small population sizes and considerable inbreeding in the history of the current population. Two obvious possibilities for “bottlenecks” in Europeans are the exodus from Africa and the repopulation of Europe after the last ice age.

A number of polymorphisms have been associated with induction of exocrine pancreatic dysfunction and/or diabetes. Some of the identified polymorphisms have been suggested in patent literature as useful in diagnosis of diabetes (see for example WO9321343 related to polymorphisms in GCK gene, and WO0023591 related to polymorphism in zsig49 gene).

Reference is also made to WO 2005/095986, directed to methods for screening test compounds for their ability to reduce retention of atherogenic lipoproteins in atherogenesis by measuring their activity as a modulator of the binding affinity of CEL (Carboxyl Ester Lipase) to various receptors, and to WO 91/18923 and U.S. Pat. No. 5,716,817 directed to DNA molecules encoding, depending on the site of action, proteins termed Bile Salt-Stimulated Lipase (BSSL) or Carboxyl Ester Lipase (CEL).

### SUMMARY OF INVENTION

### CEL Gene

The human CEL (carboxyl ester lipase) gene has been mapped to chromosome 9q34. The gene is highly polymorphic with the exon 11 VNTR varying between 3 and 28 repeats [11-13]. This exon encodes the O-glycosylation domain and the proline-rich repeats of the C-terminus. The most common allele contains 16 repeats and encodes a 745-amino acid polypeptide including a signal peptide. A polymorphism affecting the number of VNTR repeats in this exon was shown to be associated with lower low-density lipoprotein (LDL) cholesterol levels and alcohol-induced pancreatitis [14, 15]. CEL is a nonspecific lipolytic enzyme which can both hydrolyze cholesteryl esters, tri-, di-, and mono-acylglycerols as well as ceramides, phospholipids and lysophospholipids [16-18]. The enzyme may also have a role in atheromatosis[19]. Furthermore a role of CEL in the uptake of fat-soluble vitamins is being discussed. CEL is a major component of the pancreatic juice and is mainly expressed in pancreatic acinar tissue and lactating mammary glands. The gene is to a lesser extent expressed in eosinophils and endothelial cells whereas it seems not to be expressed in beta-cells.

### Exocrine Pancreatic Dysfunction:

Exocrine pancreatic dysfunction can be observed in a number of pancreatic diseases such as cystic fibrosis, pancreatitis, fibrocalculus pancreatic diabetes and hereditary pancreatitis. It is characterized by a number of symptoms including but not limited to those of or related to fecal elastase deficiency (FED), recurrent abdominal pain, pancreatic atrophy, fibrosis, fatty infiltration and lower levels of fat-soluable vitamins. A recent report indicates a prevalence of 15-30% of severe exocrine pancreas dysfunction in patients with type 1 (T1D) and type 2 diabetes (T2D)[20].

### Diabetes:

Diabetes mellitus is a heterogeneous group of metabolic diseases which is characterized by elevated blood glucose levels and increased morbidity. The endocrine cells of the pancreas which synthesize insulin and other hormones are involved in the pathogenesis of diabetes. Both genetic and environmental factors contribute to its development. The most common form is type 2 diabetes (T2D) which is characterized by defects in both insulin secretion and insulin action. In contrast type one diabetes (T1D) results from autoimmune destruction of the insulin-producing beta cells of the pancreas. Monogenic forms of diabetes account for less than 5% of the cases and are usually caused by mutations in genes associated with maturity-onset diabetes of the young (MODY).

The present invention in one aspect is directed to
1) an association of a polymorphism affecting the number of VNTR repeats of the CEL gene with a predisposition to exocrine pancreatic dysfunction and/or diabetes;
2) an association of specific haplotypes of the identified polymorphisms with a predisposition to exocrine pancreatic dysfunction and/or diabetes;
3) novel polymorphisms of the CEL gene associated with a predisposition to exocrine pancreatic dysfunction and/or diabetes;
4) a method of determining a predisposition to exocrine pancreatic dysfunction and/or diabetes comprising determining a polymorphism of the CEL gene;
5) a method of treating an individual having a predisposition to exocrine pancreatic dysfunction and/or diabetes comprising inhibiting expression of the CEL gene, said gene comprising a polymorphism described herein.

Accordingly, in the first aspect the invention relates to a method for determining a predisposition to exocrine pancreatic dysfunction and/or diabetes in a subject comprising determining in a biological sample isolated from said subject one or more polymorphisms in the CEL gene or in chromosome regions comprising the CEL gene, or in a translational or transcriptional product from said regions, said polymorphism being indicative of said predisposition.

The inventors of the present application have discovered that polymorphisms identified in the coding regions of the CEL gene are strongly associated to the presence or absence of exocrine pancreatic dysfunction and/or diabetes. Thus, detecting the presence or absence of the polymorphisms of the present invention amounts to determining a predisposition for having or not having an exocrine pancreatic dysfunction and/or diabetes. It thus follows that determining the presence of the wild-type allele amounts to determining a predisposition for not having exocrine pancreatic dysfunction and/or diabetes. The association between the presence/absence of at least one polymorphism in the above gene and the diseases is very strong.

Diagnosis of individuals for genetic predisposition to exocrine pancreatic dysfunction and/or diabetes is important so that they can be given the best treatment and adapt their lifestyle according to their genetic predisposition.

Moreover, it is expected that with the information made available by the inventors, more polymorphisms in the CEL gene will be found predisposing to exocrine pancreatic dysfunction and/or diabetes. Therefore, all polymorphisms being in linkage disequilibrium with the polymorphisms identified in the application in the chromosome regions as defined in the present application are included in the scope of the protection as diagnostic markers of the predisposition for exocrine pancreatic dysfunction and/or diabetes.

In a further aspect the invention relates to isolated oligonucleotide sequences comprising at least 10 contiguous nucleotides being 100% identical to a subsequence of the CEL gene comprising, or being adjacent to, a polymorphism of the invention, said polymorphism being associated to exocrine pancreatic dysfunction and/or diabetes.

As the present inventors have determined that the CEL gene is an etiological factor in exocrine pancreatic dysfunction and/or diabetes it is important to be able to detect and correct or suppress any polymorphism in the gene which is correlated to these diseases. The isolated oligonucleotides may be used as probes for detection of the polymorphisms and/or as primer pairs for amplification of a target nucleotide sequence and/or as part of a gene therapy vector for administration to a patient suffering from exocrine pancreatic dysfunction and/or diabetes.

In a further aspect the invention relates to a kit for predicting an increased risk of a subject of developing exocrine pancreatic dysfunction and/or diabetes or for other diagnostic and classification purposes of exocrine pancreatic dysfunction and/or diabetes comprising at least one probe comprising at least two nucleic acid sequences as defined above.

These kits which may further comprise buffers and primers and reagents can be used for diagnosing the polymorphisms and mutations which correlate to exocrine pancreatic dysfunction and/or diabetes.

The invention also relates to CEL variant proteins comprising mutations which correspond to the polymorphisms identified in the present application. These variant proteins may also be used for diagnosis of exocrine pancreatic dysfunction and/or diabetes.

According to a further aspect the invention relates to antibodies capable of selectively binding to the variant proteins as defined above with a different (such as lower or higher) binding affinity than when binding to the polypeptide having the amino acid sequence of wild type protein.

These antibodies may be used in diagnosing individuals with the polymorphisms. It is also envisaged that such specific antibodies may be used for treating patients carrying the mutated protein.

In principle, it is possible in accordance with the present invention to diagnose one or more of a) exocrine pancreatic dysfunction, b) diabetes, and c) exocrine pancreatic dysfunction and diabetes. The diagnosis can be made on the basis of a determination in a gene of the invention of one or more polymorphisms according to the present invention.

When diagnosing in a subject exocrine pancreatic dysfunction it is possible to initiate in said subject a treatment of either d) exocrine pancreatic dysfunction, e) diabetes, including a prophylactic treatment of diabetes, or f) exocrine pancreatic dysfunction and diabetes, including a prophylactic treatment of diabetes. Alternatives e) and f) can be persued when the prognostic value associated with the polymorphism indicate that the subject in question either has, or is likely to acquire, diabetes or diabetes in combination with exocrine pancreatic dysfunction.

When diagnosing in a subject diabetes it is possible to initiate in said subject a treatment of either e) diabetes, or f) exocrine pancreatic dysfunction and diabetes. Alternatives f) can be persued when the subject has developed diabetes in combination with exocrine pancreatic dysfunction.

When diagnosing in a subject both exocrine pancreatic dysfunction and diabetes it is possible to initiate in said subject a treatment of either d) exocrine pancreatic dysfunction, e) diabetes, or f) exocrine pancreatic dysfunction and diabetes.

Accordingly in further aspects the present invention relates to methods of treating patients suffering from exocrine pancreatic dysfunction and/or diabetes. Among the therapeutic methods, one method relates to a method of treating exocrine pancreatic dysfunction and/or diabetes in a subject being diagnosed as having a predisposition according to the invention, said method comprising administering to said subject a therapeutically effective amount of a gene therapy vector, comprising a nucleotide sequence capable of correcting the polymorphism or capable of suppressing the transcription and/or translation from the gene. The invention also relates to a gene therapy vector itself, said vector being capable of correcting the polymorphism in cells of a subject being diagnosed as having a predisposition according to the invention, or being capable of correcting, suppressing, supporting or changing the expression of the CEL gene in cells of a subject suffering from said diseases.

With the advent of gene therapy it has become possible to suppress and/or to eliminate the effects of a polymorphism by administering to a subject a gene therapy vector which either alters the polymorphism or suppresses the transcription and/or translation from the gene. Such gene therapy vectors have the advantage of being highly specific.

The present invention also relates to
a compound capable of inhibiting expression of the CEL gene, wherein said gene comprises a polymorphism indicative of a predisposition to exocrine pancreatic dysfunction and/or diabetes, and/or capable of inhibiting the activity of a product of said gene;
use of a compound as described above for the manufacture of a medicament for treatment of exocrine pancreatic dysfunction and/or diabetes in an individual in need thereof.
use of a compound capable of substituting for a deficiency of pancreatic enzymes and/or fat-soluble vitamins for the preparation of a medicament for the treatment of exocrine pancreatic dysfunction in a subject being diagnosed as having a predisposition to exocrine pancreatic dysfunction.
use of a compound capable of substituting for a deficiency of pancreatic enzymes and/or fat-soluble vitamins for the preparation of a medicament for the treatment of exocrine pancreatic dysfunction in a subject being diagnosed as having a predisposition to exocrine pancreatic dysfunction, wherein the compound comprises a fat-soluble vitamin selected from a group of vitamins comprising vitamins A, D, E and K.
a pharmaceutical composition for the treatment of exocrine pancreatic dysfunction and/or diabetes, comprising a compound as described above.
a method of treatment of exocrine pancreatic dysfunction and/or diabetes, comprising administering a compound or a pharmaceutical composition as above.
a method of screening for a candidate compound for therapeutic treatment of exocrine pancreatic dysfunction and/or diabetes, said method comprising an in vitro or in vivo model system comprising an exocrine pancreatic dysfunction and/or diabetes related gene wherein the gene is comprising a polymorphism associated with exocrine pancreatic dysfunction and/or diabetes,
a method for prognosis of the likelihood of development of a exocrine pancreatic dysfunction and/or diabetes comprising determining a polymorphism associated with predisposition to exocrine pancreatic dysfunction and/or diabetes,
a method of predicting the likelihood of a subject to respond to a therapeutic treatment of exocrine pancreatic dysfunction and/or diabetes, said method comprising determining the genotype of said subject in the chromosome areas comprising the CEL gene.

### FIGURE LEGENDS

FIG. 1 Pedigree of studied branches of Family 1. Severe (middle grey) and moderate (light grey) exocrine pancreatic dysfunction, and diabetes and impaired glucose tolerance (dark grey) are defined in Methods. The proband is indicated by an arrow. Abbreviations: NN, no mutation; NM, mutation.

FIG. 2 Identification of the chromosomal region and gene causing autosomal dominant diabetes and pancreatic exocrine deficiency in Family 1. (a) The results of multipoint analysis with saturation markers on chromosome 9q34 using diabetes (upper line) or elastase deficiency (lower line) as phenotype. (b) Haplotype analysis of recombinant chromosomes for individuals in Family 1 is outlined. Critical recombinant chromosomes in affected family members are presented with the borders of the disease-associated (elastase deficiency) haplotype segments indicated with arrows. (c) A genetic map of the interval on chromosome 9q34, showing the set of microsatellite markers used in refining the disease interval. (d) A physical map of the interval between D9S179 and D9S164 including known genes and their direction of transcription. The map is based on the MapViewer software. (e) The intron-exon structure of CEL. The eleven exons are shown as vertical bars. The variable number of tandem DNA repeats (VNTR) in the last exon and the patient mutations are indicated in grey colors and by the DNA sequence denominations, respectively. (f) The heterozygous frameshift mutations 1686delT in Family 1 and 1785delC in Family 2.

FIG. 3 Structure and variants of the CEL protein. (a) The full-length CEL protein with domains indicated by arrows. The numbers above the boxes denote corresponding exon numbers. The amino acids encoded by the wild-type repeat region or predicted for the mutated alleles are listed below. (b) The VNTR encoded by exon 11. Middle gray boxes illustrate the repeats of normal gene products, which are denoted “Nor” plus a suffix digit showing the number of repeats. Middle gray boxes also illustrate the upstream normal segments of deletion and insertion gene products. The latter are encoded by single-base deletions and denoted Dell (Family 1) and Del4 (Family 2), or they are encoded by single-base insertions and denoted Ins4, Ins9 and Ins11. The digits refer to the repeat number, in which the frameshifts have occurred. Dark grey boxes illustrate the novel downstream protein segments resulting from the single-base deletions. Note that the single-base insertions result in only short downstream segments (light grey boxes) before the predicted protein is truncated. The dotted boxes indicate the segments of the normal background allele, on which the single-base alterations occurred. Boxes are shown only for variants identified in Family 1 and 2 (FIG. 1 and FIG. 7, respectively). Nor4, Nor19, Nor23, Ins10 and Ins12 were identified in control subjects only. Corresponding allele frequencies of 370 control chromosomes are listed.

FIG. 4 Defining pancreatic function in affected subjects in Family 1. (a) The results from an IVGTT performed in CEL mutation carriers and controls. Open circles denote glucose-tolerant family controls (spouses) (N=9), filled circles denote glucose-tolerant mutation carriers (N=9), while filled triangles denote mutation carriers with diabetes and using only oral hypoglycemic agents and not insulin (N=5). Subjects with IGT were excluded from the analysis. Median values are shown, with bars limited by the 25^{th} and 75^{th} percentiles. The P-values given are based on calculations of the acute insulin response as described in Methods, and with values taken from Table 10. (b) Levels of fecal elastase in CEL mutation carriers and family controls (relatives without mutation) according to their age at examination of elastase levels. Mutation carriers are shown as filled circles and controls as open circles.

FIG. 5 Pancreas structure and morphology. (a) A representative tissue section from the pancreas of the deceased subject II-1. The section was stained with AB-PAS. Pronounced fibrosis was observed throughout the whole organ, surrounding regions of metaplastic, PAS-positive cells. No islet or acinar cells could be recognized. Scale bar is 200 μm. (b) Comparisons of abdominal CT scans. The pancreatic structure at the level of the splenic vein in typical representatives of a CEL mutation carrier with diabetes and pancreatic exocrine deficiency (bottom) and a mutation carrier without diabetes but with pancreatic exocrine deficiency (middle); is compared to a non-related, age- and sex-matched control (top). The pancreas is delineated by white arrows. A, G, L and S are aorta, gallbladder, liver and spleen, respectively. (c) Pancreas volume indices, i.e. estimated pancreatic volumes adjusted for the subjects' body surface area. Pooled data from Families 1 and 2 are used. Also shown is mean x-ray attenuation measured in Hounsfield Units (HU), with values equaling −1000 in air, −100 in fat, 0 in water and +1000 in bone.

FIG. 6 Studies of the mutant CEL protein. (a) A Western blot of urine specimens from subjects of Family 1. The pAbL64 antibody was used to detect CEL, the immunoreactive doublet probably representing two β-glycosylated maturation states of the same CEL gene product. Lanes: 1, a molecular weight marker; 2, two major bands corresponding to the two gene products from the alleles with Nor17 and Ins9; 3, one major band, representing two identical Nor16 alleles; 4, one major band of a diabetic mutation carrier corresponding to the normal gene product from the allele with Nor16; 5, one major band of a diabetic mutation carrier corresponding to the gene product of the allele with Ins9 (in lanes 3 and 4 no band corresponding to the mutant gene product of the Dell allele can be seen); 6, one major band corresponding to the gene product from the allele with Ins11 (no band corresponding to the mutant gene product of allele with Ins4 can be seen); 7, one major band, originating from two identical Nor14 alleles. (b) A Western blot of total protein extracted from CHO cells stably transfected with CEL constructs. The pAbL64 antibody was used to detect CEL in cell medium and cell lysate representing extracellular and intracellular fractions, respectively, of the cells. A band is visible in the lanes from wild-type, but not mutant, cells. (c) CEL stability in terms of enzyme activity as a function of time and measured at 37° C. The activity in the culture medium of non-transfected cells was negligible (not shown). (d) CEL stability in terms of enzyme activity as a function of time and measured at 4° C. (e) CEL secretion rates. Standard deviation bars and regression lines are indicated.

FIG. 7 Severe (middle grey) and moderate (light grey) exocrine pancreatic dysfunction, and diabetes or impaired glucose tolerance (dark grey) are defined in Methods. The proband is indicated by an arrow. Abbreviations: NN, no mutation; NM, mutation.

FIG. 8 The panel shows a CT scan at the level of the splenic vein in a NGT subject with FED (IV-1 of Family 1) with the normal variant Ins4 on one CEL allele and the normal variant Ins11 on the other allele (top), a diabetic subject with FED but no VNTR mutation (II-1 of Family 2; middle) and a diabetic CELmutation carrier of Family 2 (III-2; bottom). IV-1 of Family 1 had a normal pancreatic volume index of 46.1 ml/m² and a normal x-ray attenuation of 63.2 HU, II-1 of Family 2 had a normal pancreas volume index of 58.9 ml/m² and a normal x-ray attenuation of 63.2 HU, while III-2 of Family 2 had a pathological pancreas volume index of 16.0 ml/m² and a pathological x-ray attenuation of 27.6 HU. Radiological signs such as calcifications and dilation of ducts, typical findings in acute and chronic pancreatitis, are not seen. The pancreas is delineated by white arrows. A, G, L and S are aorta, gallbladder, liver and spleen, respectively.

FIG. 9 The figure shows the sequence around the polymorphism c.1175C>T in exon 9 in CEL cDNA from cultured fibroblasts from a patient (III-9) in Family 1. From genetic studies of genomic DNA in Family 1, the C allele was known to be linked to 1686delT, and subject III-9 was heterozygous for the polymorphism. Note that in the mutation carrier the two peaks at c1175C>T are of similar heights. This indicates that the mutant and normal allele are expressed at similar levels.

FIG. 10 The figure illustrates primers capable of being used in assays, as disclosed in further detail in Example 2, which provide a simple way to test for insertion or deletion variation in the first six repeats of the CEL VNTR in exon 11. The number of CEL VNTR repeats can also be determined. The method is based on duplex PCR using fluorescent primers followed by fragment analysis on a high-resolution capillary machine, for example the ABI-3730 sequencer. Insertions and/or deletions will be detected by a shift in the migration length.

FIG. 11 Illustration of FAM-blue and NED-green labelled primers binding to specific sequences within the CEL VNTR. Blue fragments (FAM-labelled primers) are illustrated in the far left hand side of the figure, while black fragments relate to NED-labelled primers (indicated by Rep 15 and Rep16 in the right hand side of the figure).

FIG. 12 A “zoom-in”-illustration of FAM-primer generated sequence data. Two narrow peaks indicates heterozygosity for 1 bp insertion or deletion. Each peak represents one repeat bound by the primer. Indications are (from left to hand as illustrated): Rep 5, 6, 7 and 8.

### DEFINITIONS

### Gene/Gene Sequence:

A compilation of:
the genomic sequences which are transcribed into a transcriptional entity
the genomic sequences in between sequences which are transcribed into a transcriptional entity (extragenic DNA)
the genomic sequences involved in regulation of expression and splicing of the gene comprising at least 2000 bp upstream and downstream from the transcribed entity.

“gene related to exocrine pancreatic dysfunction and/or diabetes” is in the present context a gene the expression of which is associated with normal and/or pathologic activity of the exocrine pancreatic system and with diabetes.

The present invention relates to the gene identified in the NCBI database (http://www.ncbi.nlm.nih.gov) as GeneID: 1056 (CEL)

Accordingly the genomic sequence of the above gene (http://genome.ucsc.edu/) is identified in the present invention as CEL gene (SEQ ID NO: 1)

“Gene of the invention” as used herein is in one embodiment the CEL gene (GeneID: 1056 (CEL)) denoted SEQ ID NO:1. Hence, a polymorphism in a “gene of the invention” can be a polymorphism in the CEL gene. In some embodiments, the polymorphism is a SNP or a DNP.

In yet another embodiment, the “gene of the invention” is a gene related to exocrine pancreatic dysfunction and/or diabetes. Hence, a polymorphism in a “gene of the invention” can be a polymorphism in any gene related to exocrine pancreatic dysfunction and/or diabetes as defined herein.

The polymorphisms according to the present invention can also be located in a chromosome region adjacent to the CEL gene. Accordingly, in one embodiment, the chromosome region extends up to 2.5 Mb on either side of the CEL gene. Hence, a “chromosome region comprising the CEL gene” can be a chromosome region extending up to 2.5 Mb on either side of the CEL gene, such as a chromosome region extending up to 2.0 Mb on either side of the CEL gene, for example a chromosome region extending up to 1.8 Mb on either side of the CEL gene, such as a chromosome region extending up to 1.6 Mb on either side of the CEL gene, for example a chromosome region extending up to 1.4 Mb on either side of the CEL gene, such as a chromosome region extending up to 1.2 Mb on either side of the CEL gene, for example a chromosome region extending up to 1.0 Mb on either side of the CEL gene, such as a chromosome region extending up to 0.75 Mb on either side of the CEL gene, for example a chromosome region extending up to 0.5 Mb on either side of the CEL gene, such as a chromosome region extending up to 0.25 Mb on either side of the CEL gene, for example a chromosome region extending up to 0.20 Mb on either side of the CEL gene, such as a chromosome region extending up to 0.10 Mb (100 kb) on either side of the CEL gene, for example a chromosome region extending up to 0.05 Mb (50 kb) on either side of the CEL gene, such as a chromosome region extending up to 0.01 Mb (10 kb) on either side of the CEL gene.

In a still further embodiment, the polymorphism according to the invention is in linkage disequilibrium with a polymorphism in the CEL gene associated with exocrine pancreatic dysfunction and/or diabetes.

The term “chromosome region containing a gene” means a part of a human chromosome containing a gene of the invention and the nucleotide sequences adjacent to both ends of the gene, i.e. SEQ ID NO: 1, wherein one end of the gene corresponds to the first nucleotide of the gene sequence, and another end corresponds to the last nucleotide of the gene sequence.

The term “adjacent” is used in connection with
(i) a gene sequence to indicate a nucleotide sequence/chromosome region that is located sufficiently close to said gene sequence in a chromosome, such as for instance less then 10 000, e.g. less than 9 000, such as less then 8 000, e.g. less than 7 000, such as less than 6 000, e.g. from 1000 to 5 000, e.g. 2 000 or 1 000 nucleotide positions. It is preferred that the adjacent region is in linkage disequilibrium with said gene sequence;
(ii) an oligonucleotide sequence to indicate that the oligonucleotide recognises a sequence that is sufficiently closely located to a specific nucleotide of interest for the oligonucleotide to be suitable for the desired detection technique, such as for instance as a primer for amplification of a target nucleotide sequence. Preferably, adjacent means less than 500, such as less than 400, e.g. less than 300, such as less than 200, e.g. less than 100, such as less than 50 nucleotide positions away from the nucleotide or nucleotide sequence of interest.

As used herein, the term “coding sequence” refers to that portion of a gene that encodes an amino acid sequence of a protein. Exons constitute the coding sequence of the gene.

Coding sequence of the above gene is identified in the present invention as SEQ ID NO: 2 (CEL)

The promoter and intron regions referred herein as the “non-coding region(s)/sequence(s)” of the given genes. As used herein, “intron” refers to a DNA sequence present in a given gene that is spliced out during mRNA maturation. The term “promoter region” refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase.

The term “fragment” when used in connection with nucleotide sequences means any fragment of the nucleotide sequence consisting of at least 20 consecutive nucleotides of that sequence.

As used herein, the term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. Such polymorphism is referred herein as “single nucleotide polymorphism” or SNP. The present invention relates to SNPs which may be an insertion, deletion and/or substitution of one nucleotide in the sequence of a gene. A polymorphic region can also be two nucleotides in length, the identity of which differs in different alleles. Such polymorphism is referred herein as “double nucleotide polymorphism” or DNP. The present invention relates to DNPs which may be an insertion, deletion and/or substitution of two nucleotides in the sequence of a gene. The present invention relates to all SNPs and DNPs which lead to a frame-shift and/or a novel or truncated protein as well as to all variations wherein the number of variant nucleotides is different from multiples of three (i.e. not insertions/deletions of 3, 6, 9, 12, 15, 21 etc. base pairs). A polymorphic region also can be several nucleotides in length. The present invention relates to polymorphisms which may be an insertion, deletion and/or substitution of one or more additional nucleotides in the sequence of a gene. A gene having at least one polymorphic region is referred as “polymorphic gene”.

Polymorphisms, which are not described in the art and do not have refSNP ID NOs in the NCBI database, are identified herein with the names indicating their location in the gene structure, for example “ex 3-1”, “prom 2” or “ex 3-3”, wherein “ex” or “prom” means the exon or promoter correspondingly, “3-1”, “2” or “3-3” indicates a particular exon or promoter of the gene. It is to be understood that the polymorphisms identified herein with the latter names are described herein for the first time,

As used herein, “allele”, which is used interchangeably herein with “allelic variant” refers to alternative forms of a gene or portions thereof. Alleles occupy the same locus or position on homologous chromosomes. When an individual has two identical alleles of a gene, the individual is said to be homozygous for the gene or allele. When an individual has two different alleles of a gene, the individual is said to be heterozygous for the gene or alleles. Alleles of a specific gene can differ from each other in a single nucleotide, or several nucleotides, and can include substitutions, deletions, and insertions of nucleotides. An allele of a gene also can be a form of a gene comprising a mutation.

As used herein, “predisposition” means that an individual having a particular genotype and/or haplotype has a higher likelihood than one not having such a genotype and/or haplotype for a particular condition/disease as one of the described herein.

As used herein, “family predisposition” means that the likelihood of having a particular disease is higher than the average in the individual's family.

As used herein, the term “haplotype” refers to a set of closely linked genetic markers present on one chromosome which tend to be inherited together (not easily separable by recombination). Some haplotypes may be in linkage disequilibrium.

As used herein, the term “genetic marker” refers to an identifiable physical location on a chromosome ((e.g., polymorphism), restriction enzyme cutting site) whose inheritance can be monitored. Markers can be expressed regions of DNA (genes) or some segment of DNA with no known coding function but whose pattern of inheritance can be determined.

As used herein, the term “linkage” refers to an association in inheritance between genetic markers such that the parental genetic marker combinations appear among the progeny more often than the non-parental.

As used herein, the term “linkage disequilibrium” (LD) means that the observed frequencies of haplotypes in a population does not agree with haplotype frequencies predicted by multiplying the frequencies of individual genetic markers in each haplotype; LD means that there exist correlations among neighbouring alleles, reflecting ‘haplotypes’ descended from single, ancestral chromosomes.

Target nucleic acid: a nucleic acid isolated from an individual and comprising or being adjacent to at least one polymorphism identified in the present invention as well as further nucleotides upstream or downstream. The target nucleic acid can be used for hybridisation, for sequencing or other analytical purposes.

Alignment: when reference is made to alignment of protein sequences alignment is carried out using the MultAlin algorithm with default settings (“Multiple sequence alignment with hierarchical clustering”, F Corpet, 1988, Nucl. Acids Res., 16 (22), 10881-10890), which is available at the internet address: http:/prodes.toulouse.inra.fr/multalin/multalin.html.

### Amino Acid Substitutions:

Substitutions within the below identified groups of amino acids are considered as conservative amino acid substitutions; substitutions of amino acids between the different groups are considered as non-conservative amino acid substitutions:

P, A, G, S, T (neutral, weakly hydrophobic)
Q, N, E, D, B, Z (hydrophilic, acid amine)
H, K, R (hydrophilic, basic)
F, Y, W (hydrophobic, aromatic)
L, I, V, M (hydrophobic)
C (cross-link forming)

### DETAILED DESCRIPTION OF THE INVENTION

### 1. Gene Polymorphism

The first aspect of the invention relates to a method for determining a predisposition to exocrine pancreatic dysfunction and/or diabetes in a subject comprising determining in a biological sample isolated from said subject two or more SNP(s) and/or DNP(s) and/or polymorphisms and/or other genomic rearrangement(s) in the CEL gene or in chromosome regions comprising the CEL gene, or in a translational or transcriptional product from said regions, said SNP(s) and/or DNP(s) and/or polymorphisms and/or other genomic rearrangement(s) being indicative of said predisposition.

In one embodiment the present invention relates to a method for determining a predisposition to exocrine pancreatic dysfunction.

In another embodiment the present invention relates to a method for determining a predisposition for diabetes.

### 1.1 Position of Polymorphisms

In one embodiment the present invention relates to one or more polymorphisms in the above identified gene, wherein the polymorphisms are located in the non-coding regions of the genes, such as an intron region or a region controlling expression of the genes, e.g. a promoter region. Such polymorphisms according to the invention may influence expression of the gene or affect the splicing or maturation of the gene-transcript, mRNA.

In another embodiment the invention relates to polymorphisms located in the coding regions of the gene, such as an exon. Such polymorphisms according to invention may lead to the production of variant proteins.

In a third embodiment the invention relates to genomic rearrangements. Such polymorphisms may consist of more than one nucleotide originating from genome regions other than the CEL gene itself, which are inserted in or connected to the CEL gene. Such polymorphisms may lead to the production of variant proteins

Variant protein's are the proteins with an amino acid sequence comprising an amino acid change, e.g. an amino acid substitution, insertion and/or deletion, which corresponds to the polymorphism of a gene. A variant protein may have an altered functional activity due to the latter polymorphism.

Thus, in one aspect the present invention relates to a method for determining a predisposition to exocrine pancreatic dysfunction and/or diabetes comprising determining one or more polymorphisms in the chromosome regions comprising the CEL gene and relating said polymorphisms to a predisposition to exocrine pancreatic dysfunction and diabetes. The one polymorphism can be located either in a coding region and/or in a non-coding region of the CEL gene. If more than one polymorphism is determined, the polymorphisms can be located either/both in a coding region and/or in a non-coding region of the CEL gene. According to these embodiments at least one polymorphism in the identified gene is to be determined. The examples of such polymorphisms are discussed below.

Thus, according to the invention determining a predisposition to exocrine pancreatic dysfunction and/or diabetes can comprise determining at least one polymorphism in the CEL gene.

In a further aspect the invention relates to determining a predisposition to exocrine pancreatic dysfunction comprising determining one or more SNP(s) and/or one or more DNP(s) in the CEL gene, or any other polymorphism which leads to a frame-shift and/or a novel or truncated protein, wherein the number of variant nucleotides is different from multiples of three (i.e. not insertion/deletion of 3, 6, 9, 12, 15, 21 etc. base pairs).

The SNP(s) and DNP(s) can be determined both alone and in any combination possible as exemplary described:

| | 1 DNP | 2 DNPs |
|---|---|---|
| 1 SNP | I | II |
| 2 SNPs | III | IV |

I: determination of one SNP or one DNP alone as well as one SNP and one DNP together or
II: one SNP alone or two DNPs together as well as one SNP and two DNPs together or
III: two SNPs together or one DNP alone as well as two SNPs and one DNP together or
IV: two SNPs together with two DNPs

Preferred polymorphisms are

ex11-1 (of the CEL gene),
ex11-2 (of the CEL gene),
ex11-3 (of the CEL gene),
ex11-4 (of the CEL gene),
ex11-5 (of the CEL gene),
ex11-6 (of the CEL gene),
ex11-7 (of the CEL gene),
ex11-8 (of the CEL gene),
ex11-9 (of the CEL gene)
and
ex11-10 (of the CEL gene).

The above polymorphisms are particular preferred when a method for determining a predisposition for exocrine pancreatic dysfunction and/or diabetes comprises determining at least one polymorphism in the CEL gene or in the chromosome regions comprising CEL gene.

Positions of the above identified polymorphisms within the coding sequences of the gene (SEQ ID NO: 2) are identified in Table 1 below:

| | in | | Nucleotide | | | | |
|---|---|---|---|---|---|---|---|
| | SEQ | | No | | Nucleo- | | |
| | ID | | (position of | | tide | SNP | No |
| Gene | NO | SNP ID | polymorphism) | SNP | No | (C/T) | Repeat |
| CEL | 2 | ex11-1 | 1686 | delT | 1719 | C | 14 |
| CEL | 2 | ex11-2 | 1785 | delC | 1719 | C | 16 |
| CEL | 2 | ex11-3 | 1951 | insC | 1719 | T | 13 |
| CEL | 2 | ex11-4 | 1984 | insC | 1719 | T | 14 |
| CEL | 2 | ex11-5 | 1984 | insC | 1719 | T | 15 |
| CEL | 2 | ex11-6 | 2017 | insC | 1719 | T | 15 |
| CEL | 2 | ex11-7 | 2017 | insC | 1719 | C | 16 |
| CEL | 2 | ex11-8 | 2050 | insC | 1719 | C | 16 |
| CEL | 2 | ex11-9 | | | 1719 | C | 16 |
| CEL | 2 | ex11-10 | | | 1719 | C | 12 |

Coding sequence of the above polymorphisms are identified in the present invention as

SEQ ID NO: 3 (ex11-1)
SEQ ID NO: 4 (ex11-2)
SEQ ID NO: 5 (ex11-3)
SEQ ID NO:6 (ex11-4)
SEQ ID NO: 7 (ex11-5)
SEQ ID NO: 8 (ex11-6)
SEQ ID NO: 9 (ex11-7)
SEQ ID NO: 10 (ex11-8)
SEQ ID NO: 11 (ex11-9)
SEQ ID NO: 12 (ex11-10)

According to the invention the above polymorphisms are genetic markers of exocrine pancreatic dysfunction and/or diabetes of the invention described below. The invention also features haplotypes of the above polymorphisms the presence of which is strongly correlated with exocrine pancreatic dysfunction and/or diabetes. Thus, the invention also relates to haplotypes which are in linkage disequilibrium.

In another aspect the invention relates to polymorphisms located in the chromosome regions comprising the above identified gene, wherein said polymorphisms are in linkage disequilibrium with at least one of the above identified polymorphisms. Thus, the invention relates to any polymorphisms in the regions of human chromosomes 9q34, comprising the CEL gene which are in linkage disequilibrium with any of the polymorphisms identified above.

The invention also includes in one embodiment any polymorphism in CEL neighbouring genes located within approximately 2.5 Mb upstream or downstream to said gene. However, the invention relates to any polymorphism of human chromosome 9q within approximately 2.5 Mb upstream or downstream of the CEL gene in case this polymorphism is in linkage disequilibrium with the CEL gene and if the polymorphism correlates with a predisposition to exocrine pancreatic dysfunction and/or diabetes or a protection against exocrine pancreatic dysfunction and/or diabetes as described in the present application. Hence, the polymorphism can be in a chromosome region extending up to 2.5 Mb on either side of the CEL gene, such as a chromosome region extending up to 2.0 Mb on either side of the CEL gene, for example a chromosome region extending up to 1.8 Mb on either side of the CEL gene, such as a chromosome region extending up to 1.6 Mb on either side of the CEL gene, for example a chromosome region extending up to 1.4 Mb on either side of the CEL gene, such as a chromosome region extending up to 1.2 Mb on either side of the CEL gene, for example a chromosome region extending up to 1.0 Mb on either side of the CEL gene, such as a chromosome region extending up to 0.75 Mb on either side of the CEL gene, for example a chromosome region extending up to 0.5 Mb on either side of the CEL gene, such as a chromosome region extending up to 0.25 Mb on either side of the CEL gene, for example a chromosome region extending up to 0.20 Mb on either side of the CEL gene, such as a chromosome region extending up to 0.10 Mb (100 kb) on either side of the CEL gene, for example a chromosome region extending up to 0.05 Mb (50 kb) on either side of the CEL gene, such as a chromosome region extending up to 0.01 Mb (10 kb) on either side of the CEL gene.

Any polymorphism of the gene being adjacent to the gene of the invention, such as polymorphisms located within the distance of 500 to 10 000 nucleotides to/from the CEL gene and which is in linkage disequilibrium with the polymorphisms identified above, is in the scope of the invention.

A polymorphism being a SNP, a DNP or any other polymorphism located within the sequence of 2000-2500 nucleotides juxtaposed to the first and/or to the last nucleotide of a genomic sequence identified herein as SEQ ID NO: 1 are preferred in one embodiment. However, polymorphisms which interact with the CEL gene are also included in the scope of the invention as indicative of the presence of a predisposition to exocrine pancreatic dysfunction and/or diabetes.

By the term “interacting gene” is meant a gene the activity of which or the activity of a product of which is dependent on the activity of the CEL gene; or a gene the activity of which or the activity of a product of which is synergistic or antagonistic with activity of the CEL gene. The interacting gene can be localised on human chromosome 9 or on any other human chromosome. The invention relates to an exocrine pancreatic and/or diabetes related gene activity, such as for example activity associated with production of enzymes for digestion or the production of hormones like insulin.

### 1.2 Products of the Genes

The invention in a further embodiment also relates to a method for determining a predisposition to exocrine pancreatic dysfunction and/or diabetes comprising determining one or more polymorphisms in the above described gene or in transcriptional or translational products of the gene.

As used herein, the term “transcriptional product of the gene” refers to an pre-messenger RNA molecule, pre-mRNA, that contains the same sequence information (albeit that U nucleotides replace T nucleotides) as the gene, or mature messenger RNA molecule, mRNA, which was produced due to splicing of the pre-mRNA, and is a template for translation of genetic information of the gene into a protein.

As used herein, the term “translational product of the gene” refers to a protein, which is encoded by the gene.

Thus, the invention includes in the scope of protection nucleic acids comprising the coding nucleotide sequences of the above gene comprising a polymorphism and proteins comprising a polymorphism corresponding to the polymorphism of the encoding nucleic acid sequence.

In particular, the invention relates to transcriptional products of the above genes being
(i) nucleic acid sequences identified in the invention as SEQ ID NO: 1 and 2, or fragments thereof,
(ii) nucleic acid sequences having at least 90%, 91%, 92%, 93%, 94%, 95%, 96%, 97%, 98% or 99% identity with SEQ ID NO: 1 and 2, or fragments thereof,
(iii) nucleic acid sequences being complementary to any of the sequences of (i) or (ii),
   said nucleic acid sequences comprising the polymorphisms of the genomic sequences described above associated with a predisposition with exocrine pancreatic dysfunction and/or diabetes.

Translational products of the genes of the invention are defined as
(i) variant proteins corresponding to the proteins identified under in the NCBI database under Ass. Nos.: NP_{—}001798 or fragments thereof, said variant proteins, fragments thereof comprising polymorphisms corresponding to the polymorphisms of the corresponding genomic sequences or transcriptional products thereof;
(ii) polypeptide sequences having at least 90%, 91%, 92%, 93%, 94%, 95%, 96%, 97%, 98% or 99% identity with the variant proteins, or fragments thereof, of (i), said polypeptide sequences comprising polymorphisms corresponding to the polymorphisms of the corresponding variant proteins.

Selected, but non-limited examples of variant proteins of the invention are given in Table 2 below:

| Gene | SEQ | SNP ID. | Protein polymorphism |
|---|---|---|---|
| CEL | 2 | ex11-1 | C563fsX673 |
| CEL | 2 | ex11-2 | C596fsX695 |
| CEL | 2 | ex11-3 | R651fsX656 |
| CEL | 2 | ex11-4 | R662fsX667 |
| CEL | 2 | ex11-5 | R662fsX667 |
| CEL | 2 | ex11-6 | R673fsX678 |
| CEL | 2 | ex11-7 | R673fsX678 |
| CEL | 2 | ex11-8 | R684fsX689 |
| CEL | 2 | ex11-9 | 16 repeats |
| CEL | 2 | ex11-10 | 12 repeats |

Amino acid sequences of the above polymorphisms are identified in the present invention as

SEQ ID NO: 13 (ex11-1)
SEQ ID NO: 14 (ex11-2)
SEQ ID NO: 15 (ex11-3)
SEQ ID NO: 16 (ex11-4)
SEQ ID NO: 17 (ex11-5)
SEQ ID NO: 18 (ex11-6)
SEQ ID NO: 19 (ex11-7)
SEQ ID NO: 20 (ex11-8)
SEQ ID NO: 21 (ex11-9)
SEQ ID NO: 22 (ex11-10)

A method for determining a predisposition to exocrine pancreatic dysfunction and/or diabetes according to the invention may include the measuring expression level of the CEL gene, such as measuring the expression level of a transcriptional product of the gene, or it may include measuring activity of another gene which is dependent on activity of a gene of the invention. For example the expression level of the CEL gene and/or the activity of the product of the CEL gene may be measured, e.g. by monitoring the cellular lipid metabolism in erythrocyte membranes or presence of CEL protein variants in urine by antibodies detecting CEL and variants of the CEL protein.

### 2. Methods of Determining Polymorphisms

### 2.1 SNP

Many methods (see Table 3 below) are known in the prior art for determining the presence of particular nucleotide sequences or for determining particular proteins having particular amino acid sequences. All of these methods may be adapted for determining the polymorphisms according to the present invention.

TABLE 3

| Method | Result |
|---|---|
| Restriction fragment length | Cleavage or non-cleavage based on |
| polymorphism | SNP results in difference in length |
| Amplified fragment | Cleavage or non-cleavage based on |
| length polymorphism | SNP results in difference in length |
| Mass spectrometry | Difference in molecular weight of hybrids |
| | between a probe and the different alleles |
| Single strand conformation | Different separation in gel based on |
| polymorphism (SSCP). SSCP | different conformation caused by single |
| heteroduplex. | nucleotide polymorphism. |
| Single nucleotide extension | Difference in signal through incorporation |
| | of differently labelled nucleotide or |
| | labelled/non-labelled nucleotide |
| Sequencing | Difference in sequence |
| Hybridisation | Hybridisation or non-hybridisation at high |
| | stringency. Often detected by using |
| | differently labelled probes. |
| Determination of Tₘ profile | difference in Tₘ profile between target |
| | and homologous vs. non-homologous |
| | probe. |
| Cleavage of single-stranded | |
| DNA | |
| Denaturing HPLC | DHPLC is based on resolving |
| | heteroduplex from homoduplex DNA |
| | fragments produced by PCR |
| | amplification using temperature- |
| | modulated heteroduplex analysis. |
| TAQMAN | PCR based technique. |

One common method for detecting polymorphisms comprises the use of a probe bound to a detectable label. By carrying out hybridisation under conditions of high stringency it is ensured that the probe only hybridises to a sequence which is 100% complementary to the probe. According to the present invention this method comprises hybridising a probe to a target nucleic acid sequence comprising at least one of the polymorphisms at the positions identified in Table 1 (see above). For other polymorphisms or mutations within the defined region, similar probes can be designed by the skilled practitioner and used for hybridisation to a target nucleic acid sequence. The design and optimisation of probes and hybridisation conditions lies within the capabilities of the skilled practitioner.

In the scope of the present invention the term “hybridisation” signifies hybridisation under conventional hybridising conditions, preferably under stringent conditions, as described for example in Sambrook et al., Molecular Cloning, A Laboratory Manual, 2nd Edition (1989) Cold Spring Harbor Laboratory Press, Cold Spring Harbor, N.Y.). The term “stringent” when used in conjunction with hybridisation conditions is as defined in the art, i.e. 15-20° C. under the melting point Tₘ, cf. Sambrook et al, 1989, pages 11.45-11.49. Preferably, the conditions are “highly stringent”, i.e. 5-10° C. under the melting point Tₘ. Under highly stringent conditions hybridisation only occurs if the identity between the oligonucleotide sequence and the locus of interest is 100%, while no hybridisation occurs if there is just one mismatch between oligonucleotide and DNA locus. Such optimised hybridisation results are reached by adjusting the temperature and/or the ionic strength of the hybridisation buffer as described in the art. However, equally high specificity may be obtained using high-affinity DNA analogues. One such high-affinity DNA analogues has been termed “locked nucleic acid” (LNA). LNA is a novel class of bicyclic nucleic acid analogues in which the furanose ring conformation is restricted in by a methylene linker that connects the 2′-O position to the 4′-C position. Common to all of these LNA variants is an affinity toward complementary nucleic acids, which is by far the highest reported for a DNA analogue (Ørum et al. (1999) Clinical Chemistry 45, 1898-1905; WO 99/14226 EXIQON). LNA probes are commercially available from Proligo LLC, Boulder, Colo., USA. Another high-affinity DNA analogue is the so-called protein nucleic acid (PNA). In PNA compounds; the sugar backbone of an oligonucleotide is replaced with an amide containing backbone, in particular an aminoethylglycine backbone. The nucleobases are retained and are bound directly or indirectly to aza nitrogen atoms of the amide portion of the backbone (Science (1991) 254: 1497-1500).

Various different labels can be coupled to the probe. Among these fluorescent reporter groups are preferred because they result in a high signal/noise ratio.

Suitable examples of the fluorescent group include fluorescein, Cy2, Cy3, Cy3.5, Cy5, Cy5.5, Cy7, acridin, Hoechst 33258, Rhodamine, Rhodamine Green, Tetramethylrhodamine, Texas Red, Cascade Blue, Oregon Green, Alexa Fluor, europium and samarium.

Another type of labels are enzyme tags. After hybridisation to the target nucleic acid sequence a substrate for the enzyme is added and the formation of a coloured product is measured. Examples of enzyme tags include a beta-Galactosidase, a peroxidase, horseradish peroxidase, a urease, a glycosidase, alkaline phosphatase, chloramphenicol acetyltransferase and a luciferase.

A further group of labels include chemiluminescent group, such as hydrazides such as luminol and oxalate esters.

A still further possibility is to use a radioisotope and detect the hybrid using scintillation counting. The radioisotope may be selected from the group consisting of ³²P, ³³P, ³⁵S, ¹²⁵I, ⁴⁵Ca, ¹⁴C and ³H.

One particularly preferred embodiment of the probe based detection comprises the use of a capture probe for capturing a target nucleic acid sequence. The capture probe is bound to a solid surface such as a bead, a well or a stick. The captured target nucleic acid sequence can then be contacted with the detection probe under conditions of high stringency and the allele be detected.

One embodiment of the probe based technique based on TAQMAN technique. This is a method for measuring PCR product accumulation using a dual-labeled fluorogenic oligonucleotide probe called a TAQMAN® probe. This probe is composed of a short (ca. 20-25 bases) oligodeoxynucleotide that is labeled with two different fluorescent dyes. On the 5′ terminus is a reporter dye and on the 3′ terminus is a quenching dye. This oligonucleotide probe sequence is homologous to an internal target sequence present in the PCR amplicon. When the probe is intact, energy transfer occurs between the two fluorophors and emission from the reporter is quenched by the quencher. During the extension phase of PCR, the probe is cleaved by 5′ nuclease activity of Taq polymerase thereby releasing the reporter from the oligonucleotide-quencher and producing an increase in reporter emission intensity.

Other suitable methods include using mass spectrometry, single base extension, determining the Tm profile of a hybrid between a probe and a target nucleic acid sequence, using single strand conformation polymorphism, using single strand conformation polymorphism heteroduplex, using RFLP or RAPD, using HPLC, using sequencing of a target nucleic acid sequence from said biological sample.

Denaturing high-performance liquid chromatography (DHPLC) has been proven useful in human and animal genetic studies for detecting single nucleotide polymorphisms (polymorphisms). In contrary to most SNP detection methods that are currently in use, SNP detection by DHPLC is not based on a re-sequencing strategy that is expensive to implement, nor does it require gel-based genotyping procedures. Instead, SNP detection by DHPLC is based on resolving heteroduplex from homoduplex DNA fragments produced by PCR amplification using temperature-modulated heteroduplex analysis.

In connection with several of these methods there is a need for amplifying the amount of target nucleic acid in the biological sample isolated from the subject. Amplification may be performed by any known method including methods selected from the group consisting of polymerase chain reaction (PCR), Ligase Chain Reaction (LCR), Nucleic Acid Sequence-Based Amplification (NASBA), strand displacement amplification, rolling circle amplification, and T7-polymerase amplification.

More particularly, PCR-based amplification can be carried out using for example a primer pair comprising appropriate sequences selected from the sequences identified in Table 4 below:

| | | | Primer |
|---|---|---|---|
| | | | SEQ ID |
| Gene | SNP | Primer | NO |
| CEL | All variants | F GTC CCT CAC TCA TTC TTC | 23 |
| | described | TAT GGC AAC | |
| | | R TCC TGC AGC TTA GCC TTG | 24 |
| | | GG | |
| | All variants | F CAC ACA CTG GGA ACC CT | 25 |
| | described | | |

F-forward PCR primer
R-reversed PCR primer
Primers for PCR: SEQ ID NO 23 and SEQ ID NO 24
Primers for Sequencing: SEQ ID NO 25 and SEQ ID NO 24

One of the primers may comprise a moiety for subsequent immobilisation of the amplified fragments.

It is understood that the primers identified above may also be used as probes for determining the polymorphisms of the invention in a nucleic acid sequence using any of the methods known in the art and featured above.

To the extent that the polymorphisms as defined in the present invention are present in DNA sequences transcribed as mRNA transcripts these transcripts constitute a suitable target sequence for detection of the polymorphisms. Commercial protocols are available for isolation of total mRNA. Through the use of suitable primers the target mRNA can be amplified and the presence or absence of polymorphisms be detected with any of the techniques described above for detection of polymorphisms in a DNA sequence.

### 3.2 Proteins

Genetic polymorphism can also be detected as a polymorphism of a protein product of the gene, or a change in a biological response like exocrine and/or endocrine pancreatic function, where the protein is involved.

For example, the genetic polymorphisms according to the present invention may influence the lipid metabolism or are linked to polymorphisms having this physiological effect, the diagnosis may also be carried out by monitoring the cellular lipid metabolism in erythrocyte membranes in a biological sample from a subject suffering from said diseases.

More particularly the lipid metabolism may be monitored by measuring the relative amount of lipids selected from the group comprising docosahexaenoic acid and arachidonic acid and the ratio between these two. It is expected that the result of a predisposing allele of a polymorphism as defined in the present invention is that the relative amount of docosahexaenoic acid is reduced and the ratio of arachidonic acid to docosahexaenoic acid is increased.

The polymorphism located in the CEL gene may also be detected by isolating a variant protein from a biological sample and determining the presence or absence of the mutated residue (according to Table 2 above) by sequencing said protein, or determining the presence or absence of another polymorphic amino acid of a variant protein by sequencing a transcriptional product of the corresponding gene. The polymorphism of any of the variant proteins of the invention may be detected likewise.

The polymorphism of a gene of the invention may also be identified by using an antibody raised against a variant protein expressed by the polymorphic gene, e.g. a variant protein of Table 2 above. By using an antibody which is able to recognise an epitope comprising a region of the variant protein comprising a polymorphism corresponding to the polymorphism of the gene it is possible to determine a predisposition of an individual to exocrine pancreatic dysfunction and/or diabetes without screening the genetic material. Thus, an antibody which is capable of specifically binding to an epitope comprising a polymorphism of the invention is also in the scope of the invention. Moreover, an antibody detecting the wild-type (normal) protein may not detect the variant protein expressed by the polymorphic gene. Thus, an antibody which is capable of specifically binding to a normal but not an epitope comprising a polymorphism of the invention is also in the scope of the invention.

Antibodies within the invention include polyclonal antibodies, monoclonal antibodies, humanized or chimeric antibodies, single chain antibodies, Fab′ fragments, F(ab′)₂ fragments, and molecules produced using a Fab expression library, and antibodies or fragments produced by phage display techniques.

Polyclonal and/or monoclonal antibodies, which are homogeneous populations of antibodies to a particular antigen, can be prepared using variant proteins (natural or recombinant) or fragment of these proteins which contain the polymorphism by standard technologies.

In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. (In the case of chickens, the immunoglobulin class can also be IgY). The hybridoma producing the mAb of this invention may be cultivated in vitro or in vivo. The ability to produce high titers of mAbs in vivo makes this the presently preferred method of production, but in some cases, in vitro production will be preferred to avoid introducing cancer cells into live animals, for example, in cases where the presence of normal immunoglobulins coming from the acitis fluids are unwanted, or in cases involving ethical considerations.

Once produced, polyclonal, monoclonal, or phage-derived antibodies are tested for specific recognition of the above described epitope by Western blot or immuno-precipitation in samples containing the polypeptides comprising the binding site or fragments thereof, e.g., as described in Ausubel et al., supra. Antibodies that specifically recognise a polymorphism of the variant protein are useful in the invention. Such antibodies can be used in an immunoassay to monitor the spectrum of the expressed protein of interest or a level of expression a variant protein in a sample collected from an individual. An antibody which is capable to inhibit an exocrine pancreatic function related activity of a variant protein is of a particular interest as a candidate compound for the treatment of exocrine pancreatic dysfunction.

The antibody may also be used in a screening assay for measuring activity of a polymorphic gene of the invention, for example as a part of a diagnostic assay. Depending on the detection technique the antibody may be coupled to a compound comprising a detectable marker. The markers or labels may be selected from any markers and labels known in the art. The antibody may also be used for determining the concentration of a substance comprising an epitope or epitope in a solution of said substance or said epitope. A wide spectrum of detection and labelling techniques is available now in the art and the techniques may therefore be selected depending on skills of the artisan practising the antibodies or on the purpose of using thereof.

In addition, techniques developed for the production of “chimeric antibodies” (Morrison et al., Proc. Natl. Aced. Sci. USA, 81:6851, 1984; Neuberger et al., Nature, 312:604, 1984; Takeda et al., Nature, 314:452, 1984) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. Nos. 4,946,778, 4,946,778, and 4,704,692) can be adapted to produce single chain antibodies against a variant protein of the invention or a fragment thereof comprising a polymorphism. Single chain antibodies are formed by linking the heavy and light chain fragments of the Fv region via an amino acid bridge, resulting in a single chain polypeptide.

Antibody fragments that recognise and bind to specific epitopes can be generated by known techniques. For example, such fragments include but are not limited to F(ab′)₂ fragments that can be produced by pepsin digestion of the antibody molecule, and Fab′ fragments that can be generated by reducing the disulfide bridges of F(ab′)₂ fragments. Alternatively, Fab′ expression libraries can be constructed (Huse et al., Science, 246:1275, 1989) to allow rapid and easy identification of monoclonal Fab′ fragments with the desired specificity.

Antibodies can be humanized by methods known in the art. For example, monoclonal antibodies with a desired binding specificity can be commercially humanized (Scotgene, Scotland; Oxford Molecular, Palo Alto, Calif.). Fully human antibodies, such as those expressed in transgenic animals are also features of the invention (Green et al., Nature Genetics 7:13-21, 1994; see also U.S. Pat. Nos. 5,545,806 and 5,569,825, both of which are hereby incorporated by reference).

Thus, isolated/identified variant proteins expressed by any of the other polymorphic genes of the invention may be used as alternative diagnostic markers of the genetic polymorphism associated with a predisposition to exocrine pancreatic dysfunction and/or diabetes.

### 4. Biological Sample

The biological sample used in the present invention may be any suitable biological sample comprising genetic material and/or proteins involved in induction of the diseases as described previously. In a preferred embodiment the sample is a blood sample, a tissue sample, a secretion sample, semen, ovum, hairs, nails, tears, urine and particular stools. The most convenient sample type is a blood sample.

### 5. Isolated Oligonucleotides

In one aspect the invention relates to an isolated oligonucleotide comprising at least 10 contiguous nucleotides being 100% identical to a subsequence of the genes of the invention comprising, or adjacent to, a polymorphism or mutation being correlated to an exocrine pancreatic dysfunction and/or diabetes related disease, or being 100% identical to a subsequence of the human genome which is in linkage disequilibrium with any of the genes of the invention comprising or adjacent to a polymorphism or mutation being correlated to exocrine pancreatic dysfunction and/or diabetes. As explained in the summary, such probes may be used for detecting the presence of a polymorphism of interest and/or they may constitute part of a primer pair and/or they may form part of a gene therapy vector used for treating exocrine pancreatic dysfunction and/or diabetes.

Preferably the isolated oligonucleotide comprises at least 10 contiguous bases of the sequence identified as SEQ ID NO: 2, or the corresponding complementary strand, or a strand sharing at least 90%, 91%, 92%, 93% or 94% sequence identity more preferably at least 95%, 96%, 97%, 98% or 99% sequence identity with SEQ ID NO: 2 or a complementary strand thereof, said isolated oligonucleotide comprising a polymorphism of the invention.

Further preferred isolated oligonucleotide may comprise at least 10 contiguous bases of any of the sequences identified as SEQ ID NO: 1 or the corresponding complementary strand thereof, or a strand sharing at least 90%, 91%, 92%, 93% or 94% sequence identity more preferably at least 95%, 96%, 97%, 98% or 99% sequence identity with any of the SEQ ID NO: 1 or a complementary strand thereof, said isolated oligonucleotide comprising a polymorphism of the invention.

These particular oligonucleotides can be used as probes for assessing the polymorphisms in the human CEL gene which is strongly correlated with exocrine pancreatic dysfunction and/or diabetes as described in this invention.

The length of the isolated oligonucleotide depends on the purpose. When being used for amplification from a sample of genomic DNA, the length of the primers should be at least 15 and more preferably even longer to ensure specific amplification of the desired target nucleotide sequence. When being used for amplification from mRNA the length of the primers can be shorter while still ensuring specific amplification. In one particular embodiment one of the pair of primers may be an allele specific primer in which case amplification only occurs if the specific allele is present in the sample. When the isolated oligonucleotides are used as hybridisation probes for detection, the length is preferably in the range of 10-15 nucleotides. This is enough to ensure specific hybridisation in a sample with an amplified target nucleic acid sequence. When using nucleotides which bind stronger than DNA (e.g. LNA and/or PNA), the length of the probe can be somewhat shorter, e.g. down to 7-8 bases.

The length can be at least 15 contiguous nucleotides, such as at least 20 nucleotides. An upper limit preferably determines the maximum length of the isolated oligonucleotide. Accordingly, the isolated oligonucleotide may be less than 1000 nucleotides, more preferably less than 500 nucleotides, more preferably less than 100 nucleotides, such as less than 75 nucleotides, for example less than 50 nucleotides, such as less than 40 nucleotides, for example less than 30 nucleotides, such as less than 20 nucleotides.

The isolated oligonucleotide can comprise from 10 to 50 nucleotides, such as from 10 to 15, from 15 to 20, from 20 to 25, or comprising from 20 to 30 nucleotides, or from 15 to 25 nucleotides.

Depending on the use the polymorphism may be located in the centre of the nucleic acid sequence, in the 5′ end of the nucleic acid sequence, or in the 3′ end of the nucleic acid sequence.

For detection based on single base extension the sequence of the oligonucleotide is adjacent to the mutation/polymorphism, either in the 3′ or 5′ direction.

The isolated oligonucleotide sequence can be complementary to a sub-sequence of the coding strand of a target nucleotide sequence or to a sub-sequence to the non-coding strand of a target nucleotide sequence as the polymorphism can be assessed with similar efficiency in the coding and the non-coding strand.

The isolated oligonucleotide sequence can be made from RNA, DNA, LNA, PNA monomers or from chemically modified nucleotides capable of hybridising to a target nucleic acid sequence. The oligonucleotides can also be made from mixtures of said monomers.

### 6. Kits

In one aspect there is provided a kit for predicting the risk of a subject for developing exocrine pancreatic dysfunction and/or diabetes, or for other diagnostic and classification purposes of said diseases, comprising at least one probe comprising a nucleic acid sequence as defined in the previous section.

In one embodiment the probe is linked to a detectable label.

In another embodiment based on single nucleotide extension the kit further comprises at least one nucleotide monomer labelled with a detectable label, a polymerase and suitable buffers and reagents.

The kit preferably also comprises set of primers for amplifying a region comprising at least one of the identified above polymorphisms in the CEL gene or transcriptional products of said gene, or the corresponding complementary strands. The primers preferably are at least 15 bases long and may be coupled to an entity suitable for subsequent immobilisation.

A kit may also comprise an antibody capable of recognising the polymorphism of the invention or capable of recognising the normal but not the polymorphism of the invention.

### 7. Exocrine Pancreatic Dysfunction and Diabetes

The invention in one embodiment relates to the association of one or more polymorphisms in the above gene, or the association of at least one of the above identified polymorphisms with a predisposition to an exocrine pancreatic dysfunction and/or diabetes.

According to the invention an association of a SNP of table 1 with a exocrine pancreatic dysfunction and/or diabetes indicates the association of expression of a particular allele of said SNP with a predisposition to said disease. Examples of protective/risky alleles of the above SNP are indicated in Table 5 below.

TABLE 5

| | |
|---|---|
| SEQ ID | Allele |

| Gene | NO | SNP No | protective | risky |
|---|---|---|---|---|
| CEL | 2 | ex11-1 | T | delT |
| CEL | 2 | ex11-2 | C | delC |
| CEL | 2 | ex11-3 | C | insC |
| CEL | 2 | ex11-4 | C | insC |
| CEL | 2 | ex11-5 | C | insC |
| CEL | 2 | ex11-6 | C | insC |
| CEL | 2 | ex11-7 | C | insC |
| CEL | 2 | ex11-8 | C | insC |
| CEL | 2 | ex11-9 | 16 repeats | <16 |
| | | | | repeats |
| CEL | 2 | ex11-10 | 16 repeats | >16 |
| | | | | repeats |

According to the invention individuals carrying the protective alleles of polymorphisms identified in the table are less likely to develop an exocrine pancreatic dysfunction and/or diabetes. In contrary, the presence of the risky allele is indicative of a predisposition to exocrine pancreatic dysfunction and/or diabetes.

Thus, in one embodiment the invention relates to a method for determining a predisposition of an individual for exocrine pancreatic dysfunction and/or diabetes, said method comprising determining at least one SNP selected from the polymorphisms identified herein as but not limited to ex11-1, ex11-2, ex11-3, ex11-4, ex11-5, ex11-6, ex11-7, ex11-8, ex11-9 and ex11-10.

In some embodiments a method for determining a predisposition to exocrine pancreatic dysfunction and/or diabetes of the invention can concern determining one or more of the polymorphisms identified in Table 1. However, in some embodiments determining a single of the above polymorphisms is sufficient for the determining a predisposition to the disease.

### 8. Medical Treatment

The present invention relates to a CEL gene associated disorder and in particular exocrine pancreatic dysfunction and/or diabetes.

Having identified a group of subjects having a polymorphism as described in the present invention, the invention also relates to the use of compounds directed to decreasing or modulating the effect of the polymorphism for the preparation of a medicament for the treatment of exocrine pancreatic dysfunction and/or diabetes in said subjects.

The compounds that bind to a CEL gene product, intracellular proteins or portions of proteins that interact with a CEL gene product, compounds that interfere with the interaction of a CEL gene product with intracellular proteins and compounds that modulate the activity of the CEL gene (i.e. modulate the level of the CEL gene expression and/or modulate the level of the CEL gene product activity) are considered to be good candidates for the manufacture of a medicament for treatment of a CEL gene associated disorder.

It is to be understood that compounds that are considered by the invention to be good candidates for the manufacture of a medicament for treatment of a CEL gene associated disorder described in the application are the compounds that can modulate the level of the polymorphic CEL gene expression and/or modulate the level of the polymorphic CEL gene product activity, wherein the polymorphism is as the described above.

Assays may additionally be utilized that identify compounds that bind to the CEL gene regulatory sequences (e.g., promoter sequences; see e.g., Platt, 1994, J. Biol. Chem. 269, 28558-28562), and that may modulate the level of CEL gene expression. Compounds may include, but are not limited to, small organic molecules, such as ones that are able to gain entry into an appropriate cell and affect expression of the CEL gene or some other gene involved in a CEL gene dependent regulatory pathway, or intracellular proteins. Such intracellular proteins may for example be involved in the control and/or regulation of the exocrine pancreatic function. Further, among these compounds are compounds that affect the level of CEL gene expression and/or the CEL gene product activity and that can be used as medicaments in the therapeutic treatment of the CEL gene associated disorders, for example exocrine pancreatic dysfunction and/or diabetes.

Compounds may include, but are not limited to, peptides such as, for example, soluble peptides, including but not limited to, Ig-tailed fusion peptides, and members of random peptide libraries; (see, e.g., Lam, et al., 1991, Nature 354, 82-84; Houghten, et al., 1991, Nature 354, 84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72, 767-778), antibodies (including, but not limited to, polyclonal, monoclonal, humanized, anti-idiotypic, chimeric or single chain antibodies, and FAb, F(ab′)₂ and Fab expression library fragments, and epitope-binding fragments thereof), and small organic or inorganic molecules. Such compounds may further comprise compounds, in particular drugs or members of classes or families of drugs, known to ameliorate or exacerbate the symptoms of exocrine pancreatic dysfunction and or diabetes, such as pancreatic enzymes and fat-soluble vitamins for exocrine pancreatic dysfunction and insulin for diabetes. Many of these drugs can be or have been used in combination.

Compounds identified via assays such as those described herein may be useful, for example, in elaborating the biological function of the CEL gene products, and for ameliorating the CEL gene associated disorders, such as exocrine pancreatic dysfunction and/or diabetes.

### Inhibitory Antisense, Ribozyme and Triple Helix Approaches

In another embodiment, symptoms of exocrine pancreatic dysfunction and/or diabetes, may be ameliorated by decreasing the level of CEL gene expression and/or the CEL gene product activity by using the CEL gene derived nucleotide sequences in conjunction with well-known antisense, gene “knock-out,” ribozyme and/or triple helix methods to decrease the level of CEL gene expression. Among the compounds that may exhibit the ability to modulate the activity, expression of the CEL gene and/or synthesis the gene products, including the ability to ameliorate the symptoms of a CEL gene disorder, are antisense, ribozyme, and triple helix molecules. Such molecules may be designed to reduce or inhibit either unimpaired, or if appropriate, mutant target gene activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of a RNA sequence, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the CEL gene could be used in an antisense approach to inhibit translation of endogenous CEL mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86, 6553-6556; Lemaitre, et al., 1987, Proc. Natl. Acad. Sci. 84, 648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6, 958-976) or intercalating agents (see, e.g., Zon, 1988, Pharm. Res. 5, 539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analogue thereof.

In yet another embodiment, the antisense oligonucleotide is an .alpha.-anomeric oligonucleotide. An alpha.-anomeric oligonucleotide forms specific double-stranded hybrids with complementary RNA in which, contrary to the usual .beta.-units, the strands run parallel to each other (Gautier, et al., 1987, Nucl. Acids Res. 15, 6625-6641). The oligonucleotide is a 2′-O-methylribonucleotide (Inoue, et al., 1987, Nucl. Acids Res. 15, 6131-6148), or a chimeric RNA-DNA analogue (Inoue, et al., 1987, FEBS Lett. 215, 327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein, et al. (1988, Nucl. Acids Res. 16, 3209), methylphosphonate oligonucleotides can be prepared by use of controlled pore glass polymer supports (Sarin, et al., 1988, Proc. Natl. Acad. Sci. U.S.A. 85, 7448-7451), etc.

While antisense nucleotides complementary to the target gene coding region sequence could be used, those complementary to the transcribed, untranslated region are most preferred.

Antisense molecules should be delivered to cells that express the target gene in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290, 304-310), the promoter contained in the 31 long terminal repeat of Rous sarcoma virus (Yamamoto, et al., 1980, Cell 22, 787-797), the herpes thymidine kinase promoter (Wagner, et al., 1981, Proc. Natl. Acad. Sci. U.S.A. 78, 1441-1445), the regulatory sequences of the metallothionein gene (Brinster, et al., 1982, Nature 296, 3942), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Thus the invention in one embodiment also encompasses vectors in which a nucleic acid sequence comprising a SNP and/or DNP and/or polymorphism and/or other genomic rearrangement related to exocrine pancreatic dysfunction and/or diabetes is cloned into the vector in reverse orientation, but operably linked to a regulatory sequence that permits transcription of antisense RNA. In this way an antisense transcript can be produced to the nucleic acid sequence comprising a SNP and/or DNP and/or polymorphism and/or other genomic rearrangement related to exocrine pancreatic dysfunction and/or diabetes, including both coding and non-coding regions.

In a further embodiment the invention also relates to vectors in which a nucleic acid sequence comprising a SNP and/or DNP and/or polymorphism and/or other genomic rearrangement in the CEL gene is cloned into the vector in reverse orientation, but operably linked to a regulatory sequence that permits transcription of antisense RNA. In this way an antisense transcript can be produced to the nucleic acid sequence comprising a SNP and/or DNP and/or polymorphism and/or other genomic rearrangement in the CEL gene, including both coding and non-coding regions.

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al., 1990, Science 247, 1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

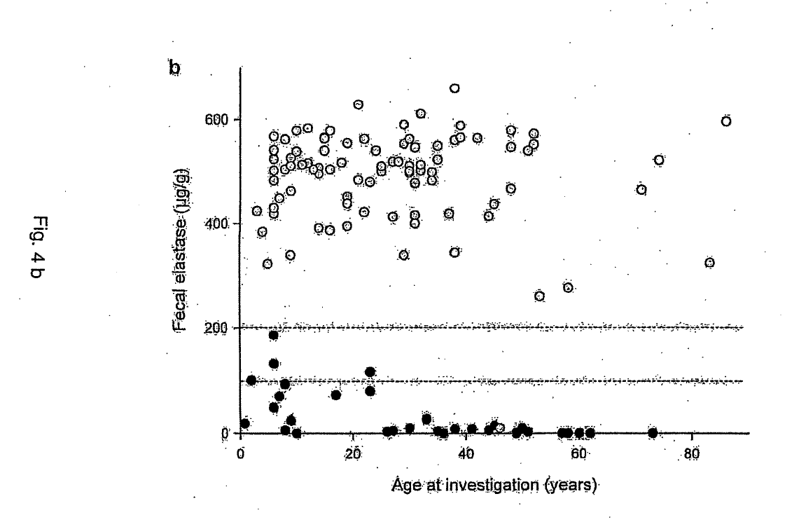

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts. For example, hammerhead ribozymes having the following sequences can be utilized. The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridizes to a target RNA sequence where after cleavage of the target RNA takes place.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6 (6), 569-584; Helene, et al., 1992, Ann. N.Y. Acad. Sci., 660, 27-36; and Maher, 1992, Bioassays 14 (12), 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC₊ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, that contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, in Section 5.9.2 that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

### Gene Therapy

Having identified polymorphism(s) as the cause of a disease it is also rendered possible in accordance with the present invention to provide a genetic therapy for subjects being diagnosed as having a predisposition for exocrine pancreatic dysfunction and/or diabetes, said therapy comprising administering to said subject a therapeutically effective amount of a gene therapy vector. The gene therapy vectors carry the protective allele of the genes. The protective allele means in the present content that expression of this allele in an individual indicates no predisposition to exocrine pancreatic dysfunction and/or diabetes. Selected, but not limited examples of protective/risky alleles of the nucleotides at positions associated with a predisposition to exocrine pancreatic dysfunction and/or diabetes are shown in Table 5.

Having discovered the CEL gene as an etiological factor in exocrine pancreatic dysfunction and/or diabetes, the present invention also provides methods for gene therapy and gene therapy vectors for use in subjects irrespective of whether they carry any of the susceptibility or protective alleles/haplotypes described in the present invention. In particular the invention relates to a gene therapy vector comprising i) a DNA sequence selected from the sequences identified as SEQ ID NO 1, or a fragment thereof, or ii) a DNA sequence selected from the sequences identified as SEQ ID NO: 2, or a fragment of said DNA sequence, wherein the DNA sequence or the fragment thereof comprises the protective allele of an SNP selected from, but not limited to, the polymorphisms identified as ex11-1, ex11-2, ex11-3, ex11-4, ex11-5, ex11-6, ex11-7, ex11-8, ex11-9 and ex11-10.

There are various different methods of gene therapy for the subjects defined in the present invention.

The first two are based on activation of the repair system of the cells by introducing into those cells a gene therapy vector which causes “correction” of the polymorphism by presenting the repair mechanism with a template for carrying out the correction. One such type includes the RNA/DNA chimeraplast, said chimeraplast being capable of correcting the polymorphism in cells of said subject. Examples of the design of such chimeraplasts can be found in e.g. U.S. Pat. No. 5,760,012; U.S. Pat. No. 5,888,983; U.S. Pat. No. 5,731,181; U.S. Pat. No. 6,010,970; U.S. Pat. No. 6,211,351.

The second method is based on application of single stranded oligonucleotides, wherein the terminal nucleotides is protected from degradation by using 3′ and 5′ phosphorothioat-linkage of the monomers. This gene therapy vector is also capable of “correcting” the polymorphism by replacing one nucleotide with another.

These first two types of gene therapy vectors comprise a small sequence (less than 50 bases) which overlaps with the polymorphism in question. Suitable sequences for this purpose are genomic sequences located around the polymorphism.

Other types of gene therapy include the use of retrovirus (RNA-virus). Retrovirus can be used to target many cells and integrate stably into the genome. Adenovirus and adeno-associated virus can also be used. A suitable retrovirus or adenovirus for this purpose comprises an expression construct with the wildtype gene under the control of the wildtype promoter or a constitutive promoter or a regulatable promoter such as a repressible and/or inducible promoter or a promoter comprising both repressible and inducible elements.

A further group of gene therapy vectors includes vectors comprising interfering RNA (RNAi) for catalytic breakdown of mRNA carrying the polymorphism. RNAi can be used for lowering the expression of a given gene for a relatively short period of time. In particular these RNAi oligos may be used for therapy for both subjects carrying a susceptibility allele as described in the present invention as well as for subjects which do not carry such an allele.

Interfering RNA (“RNAi”) is double stranded RNA that results in catalytic degradation of specific mRNAs, and can also be used to lower gene expression.

Described below are methods and compositions whereby a CEL gene disorder, in particular exocrine pancreatic dysfunction and/or diabetes, may be treated.

With respect to an increase in the level of normal CEL gene expression and/or CEL gene product activity, the CEL gene derived nucleotide sequences, for example, be utilized for the treatment of a CEL gene associated disorder such as exocrine pancreatic dysfunction and/or diabetes. Such treatment can be performed, for example, in the form of gene replacement therapy. Specifically, one or more copies of a normal CEL gene or a portion of said gene that directs the production of a gene product exhibiting normal CEL gene function, may be inserted into the appropriate cells within a patient, using vectors that include, but are not limited to adenovirus, adeno-associated virus, and retrovirus vectors, in addition to other particles that introduce DNA into cells, such as liposomes.

Gene replacement therapy techniques should be capable delivering the CEL gene sequences to cells expressing the corresponding gene within patients. Thus, in one embodiment, techniques that are well known to those of skill in the art (see, e.g., PCT Publication No. WO89/10134, published Apr. 25, 1988) can be used to enable the CEL gene sequences to be uptaken by the cells. Viral vectors may advantageously be used for the purpose. Also included are methods using liposomes either in vivo ex vivo or in vitro, wherein the CEL gene sense or antisense DNA is delivered to the cytoplasm and nucleus of target cells. Liposomes can deliver the CEL gene sense or nonsense RNA to humans and the lungs or skin through intrathecal delivery either as part of a viral vector or as DNA conjugated with nuclear localizing proteins or other proteins that increase take up into the cell nucleus.

In another embodiment, techniques for delivery involve direct administration of such CEL gene sequences to the site of the cells in which the CEL gene sequences are to be expressed, in particular the pancreatic acinar tissue and lactating mammary glands. Additional methods that may be utilized to increase the overall level of the CEL gene expression and/or the CEL gene product activity include the introduction of appropriate CEL gene-expressing cells, preferably autologous cells, into a patient at positions and in numbers that are sufficient to ameliorate the symptoms of a CEL gene associated disorder, such as exocrine pancreatic dysfunction and/or diabetes. Such cells may be either recombinant or non-recombinant.

Among the cells that can be administered to increase the overall level of CEL gene expression in a patient are normal cells, preferably brain cells and also choroid plexus cells within the CNS which are accessible through intrathecal injections. Alternatively, cells, preferably autologous cells, can be engineered to express CEL gene sequences, and may then be introduced into a patient in positions appropriate for the amelioration of the symptoms of a CEL gene associated disorder. Alternately, cells that express an unimpaired CEL gene and that are from a MHC matched individual can be utilized, and may include, for example, brain cells. The expression of the CEL gene derived sequences is controlled by the appropriate gene regulatory sequences to allow such expression in the necessary cell types. Such gene regulatory sequences are well known to the skilled artisan. Such cell-based gene therapy techniques are well known to those skilled in the art, see, e.g., Anderson, U.S. Pat. No. 5,399,349.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds, such as those identified via techniques such as those described above that are capable of modulating the CEL gene product activity can be administered using standard techniques that are well known to those of skill in the art.

### 10. Drug Discovery

A cell line based on cells isolated from a subject carrying a polymorphism according to the invention can also be cultured and used for the screening purposes.

A vector can comprise part(s) of the nucleotide sequence of SEQ ID NO: 1, or SEQ ID NO: 2, said sequence comprising a polymorphism associated with exocrine pancreatic dysfunction and/or diabetes. Using a vector comprising SEQ ID NO: 1 more precisely mimics the expression in vivo due to the presence of introns and possibly the native promoter of the genes.

According to some embodiments the vector may comprise a constitutive promoter. According to other embodiments the vector may comprise a promoter sequence comprising a regulatable promoter such as a viral promoter sequence.

The vector may be transferred into a host cell which can be used for screening purposes in drug discovery. The host cells may be selected from a bacterial cell, a yeast cell, a mammalian cell line, more preferably a human cell line. More preferably, the host cell is a human immortalised cell line such as human melanocyte.

Screening of compounds for a functionality related to exocrine pancreatic dysfunction and/or diabetes can be carried out by exposing a cell as described above to a drug candidate and measuring a response related to the exocrine pancreatic function.

The response may for example be a change in the lipid metabolism which for example may be monitored by measuring the relative amount of lipids selected from the group comprising docosahexaenoic acid and arachidonic acid and the ratio between these two.

Screening methods for compounds with are capable of modulating the CEL protein-protein interactions are within the scope of the invention.

For the purpose of below discussion molecules that produced in the cells due to activity of the CEL gene, such as transcriptional and translational products of the gene, are termed herein “gene products”, if not specified otherwise.

Any method suitable for detecting protein-protein interactions may be employed for identifying the CEL protein-protein interactions.

Among the traditional methods that may be employed are co-immunoprecipitation, cross-linking and co-purification through gradients or chromatographic columns. Utilizing procedures such as these allows for the identification of proteins, including intracellular proteins, which interact with CEL proteins. Once isolated, such a protein can be identified and can be used in conjunction with standard techniques, to identify proteins it interacts with. For example, at least a portion of the amino acid sequence of a protein that interacts with the CEL protein can be ascertained using techniques well known to those of skill in the art, such as via the Edman degradation technique (see, e.g., Creighton, 1983, “Proteins: Structures and Molecular Principles,” W.H. Freeman & Co., N.Y., pp. 34-49). The amino acid sequence obtained may be used as a guide for the generation of oligonucleotide mixtures that can be used to screen for gene sequences encoding such proteins. Screening made be accomplished, for example, by standard hybridization or PCR techniques. Techniques for the generation of oligonucleotide mixtures and the screening are well-known. (See, e.g., Ausubel, supra, and 1990, “PCR Protocols: A Guide to Methods and Applications,” Innis, et al., eds. Academic Press, Inc., New York).

Additionally, methods may be employed that result in the simultaneous identification of genes that encode a protein which interacts with the CEL protein. These methods include, for example, probing expression libraries with labelled CEL polypeptides, using CEL proteins in a manner similar to the well known technique of antibody probing of lambda.gtll and lambda.gt10 libraries.

One method that detects protein interactions in vivo, the two-hybrid system, is described in detail for illustration only and not by way of limitation. One version of this system has been described (Chien, et al., 1991, Proc. Natl. Acad. Sci. USA, 88, 9578-9582) and is commercially available from Clontech (Palo Alto, Calif.).

Briefly, utilizing such a system, plasmids are constructed that encode two hybrid proteins: one consists of the DNA-binding domain of a transcription activator protein fused to the CEL gene peptide product and the other consists of the transcription activator protein's activation domain fused to an unknown protein that is encoded by a cDNA that has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, CEL gene derived peptide products may be used as the bait gene product. Total genomic or cDNA sequences are fused to the DNA encoding an activation domain. This library and a plasmid encoding a hybrid of a bait CEL protein, or a fragment thereof, fused to the DNA-binding domain are co-transformed into a yeast reporter strain, and the resulting transformants are screened for those that express the reporter gene. For example, and not by way of limitation, a bait CEL gene sequence, such as the open reading frame of the CEL gene, can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait CEL gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait CEL gene sequence-GAL4 fusion plasmid into a yeast strain that contains a lacZ gene driven by a promoter that contains GAL4 activation sequence. A cDNA encoded protein, fused to GAL4 transcriptional activation domain, that interacts with bait CEL gene product will reconstitute an active GAL4 protein and thereby drive expression of the HIS3 gene. Colonies that express HIS3 can be detected by their growth on petri dishes containing semi-solid agar based media lacking histidine. The cDNA can then be purified from these strains, and used to produce and isolate the bait CEL protein-interacting protein using techniques routinely practiced in the art.

The invention also relates to screening assays for compounds that interfere with the CEL gene products macromolecule interaction.

The CEL gene products of the invention may, in vivo, interact with one or more macromolecules, including intracellular macromolecules, such as proteins. Such macromolecules may include, but are not limited to, nucleic acid molecules and those proteins identified via methods such as those described above. For purposes of this discussion, the macromolecules are referred to herein as “binding partners”. Compounds that are able to disrupt the CEL gene products binding in this way may be useful in regulating the activity of products of the CEL gene, especially variant CEL proteins and thereof derived peptide products. Such compounds may include, but are not limited to molecules such as peptides, and the like, which would be capable of gaining access to a CEL gene product.

The basic principle of the assay systems used to identify compounds that interfere with the interaction between CEL gene products and their binding partner or partners involves preparing a reaction mixture containing the CEL gene product, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of CEL gene product and its binding partner. Control reaction mixtures are incubated without the test compound or with a placebo. The formation of any complexes between the CEL gene product and the binding partner is then detected. The formation of a complex in the control reaction, but not in the reaction mixture containing the test compound, indicates that the compound interferes with the interaction of the CEL gene product and the interactive binding partner. Additionally, complex formation within reaction mixtures containing the test compound and for example normal (wild type) CEL protein may also be compared to complex formation within reaction mixtures containing the test compound and a variant CEL protein. This comparison may be important in those cases wherein it is desirable to identify compounds that disrupt interactions of mutant but not wild type CEL protein.

The assay for compounds that interfere with the interaction of CEL gene products and their binding partners can be conducted in a heterogeneous or homogeneous format. Heterogeneous assays involve anchoring either the CEL gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the CEL gene products and the binding partners, e.g., by competition, can be identified by conducting the reaction in the presence of the test substance; i.e., by adding the test substance to the reaction mixture prior to or simultaneously with the CEL gene protein and interactive intracellular binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the CEL gene product or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the CEL gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labelled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of a CEL gene product and the interactive binding partner is prepared in which either the CEL gene product or its binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances that disrupt the CEL gene product/binding partner interaction can be identified.

In another embodiment, the CEL gene product can be prepared for immobilization using recombinant DNA techniques. For example, the CEL gene coding region can be fused to the glutathione-S-transferase (GST) gene using a fusion vector, such as pGEX-5X-1, in such a manner that its binding activity is maintained in the resulting fusion protein. The interactive binding partner can be purified and used to raise an antibody, using methods routinely practiced in the art. The antibody can then be labeled with a radioactive isotope such as ¹²⁵I, for example, by methods routinely practiced in the art. In a heterogeneous assay, e.g., the GST-CEL fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the CEL gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-CEL fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the CEL gene product/binding partner interaction can be detected by adding the labelled antibody and measuring the radioactivity associated with the beads.

In still another embodiment of the invention, these same techniques can be employed using peptide fragments that correspond to the binding domains of CEL proteins and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this Section above, and allowed to interact with and bind to its labeled binding partner, which has been treated with a proteolytic enzyme, such as trypsin. After washing, a short, labelled peptide comprising the binding domain may remain associated with the solid material, which can be isolated and identified by amino acid sequencing. Also, once the gene coding for the segments can be engineered to express peptide fragments of the protein, which can then be tested for binding activity and purified or synthesized.

For example, and not by way of limitation, a CEL gene product can be anchored to a solid material as described above by making a GST-CEL fusion protein and allowing it to bind to glutathione agarose beads. The interactive binding partner obtained can be labeled with a radioactive isotope, such as ³⁵S, and cleaved with a proteolytic enzyme such as trypsin. Cleavage products can then be added to the anchored GST-CEL fusion protein and allowed to bind. After washing away unbound peptides, labelled bound material, representing the binding partner binding domain, can be eluted, purified, and analyzed for amino acid sequence by well-known methods. Peptides so identified can be produced synthetically or fused to appropriate facilitative proteins using recombinant DNA technology.

The invention also provides assays for identification of compounds that ameliorate the CEL gene associated disorders, such as exocrine pancreatic dysfunction and/or diabetes.

Compounds, including but not limited to binding compounds identified via assay techniques such as those described above can be tested for the ability to ameliorate symptoms of a CEL gene associated disorder including exocrine pancreatic dysfunction and/or diabetes.

It should be noted that the assays described herein can identify compounds that affect the CEL gene activity by either affecting CEL gene expression or by affecting the level of CEL gene product activity. For example, compounds may be identified that are involved in another step in the pathway in which the CEL gene and/or the CEL gene product is involved and, by affecting this same pathway may modulate the effect of the CEL gene on the development of exocrine pancreatic dysfunction and/or diabetes. Such compounds can be used as part of a therapeutic method for the treatment of the disorder.

Described below are cell-based and animal model-based assays for the identification of compounds exhibiting such an ability to ameliorate symptoms of the CEL gene activity associated with exocrine pancreatic dysfunction and/or diabetes.

First, cell-based systems can be used to identify compounds that may act to ameliorate symptoms of a CEL gene associated disorder like exocrine pancreatic dysfunction and/or diabetes. Such cell systems can include, for example, recombinant or non-recombinant cell, such as cell lines, that express the CEL gene.

In utilizing such cell systems, cells that express the CEL gene may be exposed to a compound suspected of exhibiting an ability to ameliorate symptoms of a CEL gene disorder, such as exocrine pancreatic dysfunction and/or diabetes, at a sufficient concentration and for a sufficient time to elicit such an amelioration of such symptoms in the exposed cells. After exposure, the cells can be assayed to measure alterations in the expression of the CEL gene, e.g., by assaying cell lysates for the presence of CEL gene transcripts (e.g., by Northern analysis) or for the CEL gene translation products expressed by the cell. Compounds that modulate expression of the CEL gene are considered to be good candidates as therapeutics.

Alternatively, the cells are examined to determine whether one or more cellular phenotypes associated with a CEL gene disorder, such as exocrine pancreatic dysfunction and/or diabetes, has been altered to resemble a more normal or unimpaired, unaffected phenotype, or a phenotype more likely to produce a lower incidence or severity of disorder symptoms.

In addition, animal-based systems or models for a CEL gene associated disorder, such as exocrine pancreatic dysfunction and/or diabetes, which may include, for example mice, may be used to identify compounds capable of ameliorating symptoms of the disorder. Such animal models may be used as test substrates for the identification of drugs, pharmaceuticals, therapies and interventions that may be effective in treating such disorders. For example, animal models may be exposed to a compound suspected of exhibiting an ability to ameliorate symptoms, at a sufficient concentration and for a sufficient time to elicit such an amelioration of symptoms of a CEL gene associated disorder, such as exocrine pancreatic dysfunction and/or diabetes, in the exposed animals. The response of the animals to the exposure may be monitored by assessing the reversal of such symptoms.

With regard to intervention, any treatments that reverse any aspect of symptoms of a CEL gene associated disorder, such as exocrine pancreatic dysfunction and/or diabetes, should be considered as candidates for human therapeutic intervention in such a disorder. In particular, the invention concerns candidate compounds capable of
i) modulating expression of the CEL gene and/or a gene related to exocrine pancreatic dysfunction and/or diabetes, said compound being selected from an isolated antisense nucleotide sequence or an nucleotide sequence complementary to the regulatory region of said gene, said nucleotide sequence being capable of forming triple helix structures that prevent transcription of said gene, and/or
ii) modulating activity of a transcriptional product of the CEL gene, said transcriptional product being (1) a nucleotide sequence selected from SEQ ID NO: 1, (2) a sequence having at least 90%, 91%, 92%, 93%, 94%, 96%, 97%, 98% or 99% sequence identity with SEQ ID NO: 1, or a fragment thereof, and/or (3) a sequence complementary to one of these sequences or a fragment thereof,
wherein said candidate compound is preferably selected from an isolated antisense sequence or a ribozyme molecule, and/or
iii) modulating activity of translational products of the CEL gene, said translational products being variant proteins discussed above,
wherein said candidate compound is preferably selected from an antibody molecule against said translational product, or a molecule capable of interfering with biological activity of said translational product.

The term “modulating” is meant in the present context both inhibiting and stimulating

Accordingly, in another embodiment the invention relates to a compound with is capable of directly or indirectly modulate the activity of a gene interacting with the CEL gene.

The invention further relates to a pharmaceutical composition comprising a compound of the invention.

### 11. Pharmaceutical Composition

Once the candidate compound(s) of the invention has been identified it is further within the scope of the invention to provide a pharmaceutical composition comprising one or more compound(s). In the present context the term pharmaceutical composition is used synonymously with the term medicament.

The invention is further related to a pharmaceutical composition capable of preventing the symptoms of a CEL gene associated disorder, such as exocrine pancreatic dysfunction and/or diabetes, said composition comprising an effective amount of one or more of the compounds described above. The pharmaceutical composition may further comprise compounds, in particular drugs or members of classes or families of drugs, known to ameliorate or exacerbate the symptoms of exocrine pancreatic dysfunction and/or diabetes, with the use of exocrine pancreatic enzymes and fat soluble vitamins for exocrine pancreatic dysfunction and insulin for diabetes. The medicament of the invention may also comprise an effective amount of one or more of the compounds as defined above in combination with pharmaceutically acceptable additives.

Formulations of the compounds of the invention can be prepared by techniques known to the person skilled in the art. The formulations may contain pharmaceutically acceptable carriers and excipients including microspheres, liposomes, microcapsules, nanoparticles or the like.

The preparation may suitably be administered by injection, optionally at the site, where the active ingredient is to exert its effect. Additional formulations which are suitable for other modes of administration include suppositories, nasal, pulmonal and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

Other formulations are such suitable for nasal and pulmonal administration, e.g. inhalators and aerosols.

The active compound may be formulated as neutral or salt forms. Pharmaceutically acceptable salts include acid addition salts (formed with the free amino groups of the peptide compound) and which are formed with inorganic acids such as, for example, hydrochloric or phosphoric acids, or such organic acids as acetic acid, oxalic acid, tartaric acid, mandelic acid, and the like. Salts formed with the free carboxyl group may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg active ingredient per administration with a preferred range of from about 0.1 μg to 5000 μg per kilo body weight. Using monomeric forms of the compounds, the suitable dosages are often in the range of from 0.1 μg to 5000 μg per kilo body weight, such as in the range of from about 0.1 μg to 3000 μg per kilo body weight, and especially in the range of from about 0.1 μg to 1000 μg per kilo body weight. Using multimeric forms of the compounds, the suitable dosages are often in the range of from 0.1 μg to 1000 μg per kilo body weight, such as in the range of from about 0.1 μg to 750 μg per kilo body weight, and especially in the range of from about 0.1 μg to 500 μg per kilo body weight such as in the range of from about 0.1 μg to 250 μg per kilo body weight. In particular, when administering nasally smaller dosages are used than when administering by other routes. Administration may be performed once or may be followed by subsequent administrations. The dosage will also depend on the route of administration and will vary with the age and weight of the subject to be treated. A preferred dosage of multimeric forms would be in the interval 1 mg to 70 mg per 70 kg body weight.

For some indications a localised or substantially localised application is preferred.

For other indications, intranasal application is preferred.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promotes delivery of the active substance to its target.

In many instances, it will be necessary to administrate the formulation multiple times. Administration may be a continuous infusion, such as intraventricular infusion or administration in more doses such as more times a day, daily, more times a week, weekly, etc. It is preferred that administration of the medicament is initiated before or shortly after the individual has been subjected to the factor(s) that may lead to development of exocrine pancreatic dysfunction and/or diabetes. Preferably the medicament is administered within 8 hours from the factor onset, such as within 5 hours from the factor onset. Many of the compounds exhibit a long term effect whereby administration of the compounds may be conducted with long intervals, such as 1 week or 2 weeks.

In another aspect the invention relates to a process of producing a pharmaceutical composition, comprising mixing an effective amount of one or more of the compounds of the invention, or a pharmaceutical composition according to the invention with one or more pharmaceutically acceptable additives or carriers, and administer an effective amount of at least one of said compound, or said pharmaceutical composition to a subject.

In yet a further aspect the invention relates to a method of treating an individual suffering from one or more of the diseases discussed above by administering the said individual a compound as described herein or a pharmaceutical composition comprising said compound.

### 12. Therapeutic and Diagnostic Methods

As already discussed above, information provided by the present invention is to be used for diagnostic and therapeutic purposes.

In one embodiment the invention relates to a method for determining a predisposition for exocrine pancreatic dysfunction in a subject comprising determining in a biological sample isolated from said subject one or more SNP(s) and/or one or more DNP(s) and/or genomic rearrangement(s) in a) the CEL gene and/or b) in chromosome regions comprising said CEL gene, and/or c) in transcriptional products comprising said one or more SNP(s) and/or one or more DNP(s), and/or d) translational products arising from said transcriptional products, or comprising determining two or more polymorphisms in the CEL gene or in a translational or transcriptional product of said gene, preferably determining the presence of an SNP(s) according to Table 1.

In a second embodiment the invention relates to a method for determining a predisposition for exocrine pancreatic dysfunction in a subject comprising determining in a biological sample isolated from said subject one or more polymorphisms in a) the CEL gene and/or b) in chromosome regions comprising said CEL gene, and/or c) in transcriptional products comprising said one or more polymorphisms, and/or d) translational products arising from said transcriptional products, or comprising determining two or more polymorphisms in the CEL gene or in a translational or transcriptional product of said gene, preferably determining the presence of an SNP(s) according to Table 1.

In another embodiment the invention relates to a method for determining a predisposition for not having exocrine pancreatic dysfunction and/or diabetes in a subject comprising determining in a biological sample isolated from said subject a protective allele of a polymorphism in the CEL gene which is associated with exocrine pancreatic dysfunction and/or diabetes, preferably a protective allele of a SNP(s) according to Table 1 and Table 5.

In still another embodiment the invention relates to a method for determining a protection against exocrine pancreatic dysfunction and/or diabetes, in a subject comprising determining in a biological sample isolated from said subject a protective allele of an SNP(s) selected form the SNP(s) ex11-1, ex11-2, ex11-3, ex11-4, ex11-5, ex11-6, ex11-7, ex11-8, ex11-9 and ex11-10.

Further, the invention relates to a method for evaluating the likelihood of the development of exocrine pancreatic dysfunction and/or diabetes comprising determining a polymorphism in a chromosome region comprising the CEL gene, said polymorphism being preferably an SNP associated with exocrine pancreatic dysfunction and/or diabetes as selected from the polymorphisms discussed above.

A method for evaluating the likelihood of the development of exocrine pancreatic dysfunction and/or diabetes comprising determining a polymorphism in the CEL gene, wherein the polymorphism is an SNP selected from the polymorphisms discussed above, is also in the scope of the invention.

Other embodiments of the invention relate to methods for treatment of exocrine pancreatic dysfunction and/or diabetes, in a subject being diagnosed as having a predisposition according to the invention, comprising
1) administering to said subject a therapeutically effective amount of a gene therapy vector, said gene therapy vector comprising the protective allele of an SNP associated with exocrine pancreatic dysfunction and/or diabetes (discussed above), and/or
2) administering to said subject a therapeutically effective amount of a candidate drug compound of the invention (discussed above) or a pharmaceutical composition comprising thereof.

The invention also relates to a method for predicting the likelihood of a subject to respond to a therapeutic treatment of exocrine pancreatic dysfunction and/or diabetes, said method comprising determining the genotype of said subject in the chromosome areas comprising the CEL gene.

With the knowledge of the present invention it is possible to design pharmaceutical treatment of the diagnosed subjects more precisely, because pharmaceuticals can be designed and used to decrease the expression of the genes and thus decrease the effect of the gene polymorphism. Thus, a patient having exocrine pancreatic dysfunction and/or diabetes described in the application may be more effectively and without undesirable side effects treated.

### 13 Human Identification

In addition to their value related to exocrine pancreatic dysfunction and/or diabetes and associated pathologies, the SNPs provided by the present invention are also valuable as human identification markers for such applications as forensics and paternity testing. Genetic variations in the nucleic acid sequences between individuals can be used as genetic markers to identify individuals and to associate a biological sample with an individual. Determination of which nucleotides occupy a set of SNP positions in an individual identifies a set of SNP markers that distinguishes the individual. The more SNP positions are analyzed, the lower the probability that the set of SNPs in one individual is the same as that in an unrelated individual. Preferably, if multiple sites are analyzed, the sites are unlinked. Thus, SNPs of the invention may be used in conjunction with polymorphisms in distal genomic regions.

SNPs have numerous advantages over other types of polymorphic markers, such as short tandem repeats (STRs), and therefore SNPs are the preferred markers for forensic and human identification applications. SNPs can be easily scored and are amenable to automation, making SNPs the markers of choice for large-scale forensic databases. SNPs are found in much greater abundance throughout the genome than repeat polymorphisms. Population frequencies of two polymorphic forms can usually be determined with greater accuracy than those of multiple polymorphic forms at multi-allelic loci. SNPs are mutationaly more stable than repeat polymorphisms. SNPs are not susceptible to artifacts such as stutter bands that can hinder analysis. Stutter bands are frequently encountered when analyzing repeat polymorphisms, and are particularly troublesome when analyzing samples such as crime scene samples that may contain mixtures of DNA from multiple sources. Another significant advantage of SNP markers over STR markers is the much shorter length of nucleic acid needed to score a SNP. For example, STR markers are generally several hundred base pairs in length. SNPs, on the other hand, comprise a single base pair, and generally a short conserved region on either side of the SNP position for primer and/or probe binding. This makes SNPs more amenable to typing in highly degraded or aged biological samples that are frequently encountered in forensic casework in which DNA may be fragmented into short pieces. SNPs also are not subject to microvariant and “off-ladder” alleles frequently encountered when analyzing STR loci. Microvariants are deletions or insertions within a repeat unit that change the size of the amplified DNA product so that the amplified product does not migrate at the same rate as reference alleles with normal sized repeat units. When separated by size, such as by electrophoresis on a polyacrylamide gel, microvariants do not align with a reference allelic ladder of standard sized repeat units, but rather migrate between the reference alleles. The reference allelic ladder is used for precise sizing of alleles for allele classification; therefore alleles that do not align with the reference allelic ladder lead to substantial analysis problems. Furthermore, when analyzing multi-allelic repeat polymorphisms, occasionally an allele is found that consists of more or less repeat units that have been previously seen in the population. These alleles will migrate outside the size range of known alleles in a reference allelic ladder, and therefore are referred to as “off-ladder” alleles. In extreme cases, the allele may contain so few or so many repeats that it migrates well out of the range of the reference allelic ladder. In this situation, the allele may not even be observed, or, with multiplex analysis, it may migrate within or close to the size range for another locus, further confounding analysis. SNP analysis avoids the problems of microvariants and off-ladder alleles encountered in STR analysis. Importantly, microvariants and off-ladder alleles may provide significant problems, and may be completely missed, when using analysis methods such as oligonucleotide hybridization arrays, which utilize oligonucleotide probes specific for certain known alleles. Furthermore, off-ladder alleles and microvariants encountered with STR analysis, even when correctly typed, may lead to improper statistical analysis, since their frequencies in the population are generally unknown or poorly characterized, thereby the statistical significance of a matching genotype may be questionable. All these advantages are considerable in light of the consequences of most DNA identification cases, which may lead to life imprisonment for an individual, or re-association of remains to the family of a deceased individual.

DNA can be isolated from biological samples such as blood, bone, hair, saliva, and semen and compared with the DNA from a reference source at particular SNP positions. Multiple SNP markers can be assayed simultaneously in order to increase the power of discrimination and the statistical significance of a matching genotype. For example, oligonucleotide arrays can be used to genotype a large number of SNPs simultaneously. The SNPs provided by the present invention can be assayed in combination with other polymorphic genetic markers, such as other SNPs or short tandem repeats (STRs), in order to identify an individual or to associate an individual with a particular biological sample.

Furthermore, the SNPs provided by the present invention can be typed for inclusion in a database of DNA genotypes, for example a criminal DNA databank. A genotype obtained from a biological sample of unknown source can then be queried against the database to find a matching genotype, with the SNPs of the present invention providing nucleotide positions at which to compare the known and unknown DNA sequences for identity.

SNPs of the present invention can also be assayed for use in paternity testing. The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child. If the set of polymorphisms in the child attributable to the father does not match the set of polymorphisms of the putative father, it can be concluded, barring experimental error, that the putative father is not the real father. If the set of polymorphisms in the child attributable to the father does match the set of polymorphisms of the putative father, a statistical calculation can be performed to determine the probability of coincidental match.

The use of the SNPs of the present invention for human identification extends to various authentication systems, commonly referred to as biometric systems. Biometric systems convert physical characteristics of humans (or other organisms) into digital data for precise quantification. Biometric systems include various technological devices that measure such unique anatomical or physiological characteristics as finger, thumb, or palm prints; hand geometry, vein patterning on the back of the hand; blood vessel patterning of the retina and color and texture of the iris; facial characteristics; voice patterns; signature and typing dynamics; and DNA. Such physiological measurements can be used to verify identity and restrict or allow access based on the identification. Examples of applications for biometrics include physical area security, computer and network security, aircraft passenger check-in and boarding, financial transactions, medical records access, government benefit distribution, voting, law enforcement, passports, visas and immigration, prisons, various military applications, and for restricting access to expensive or dangerous items, such as automobiles or guns. For a further review of biometric systems, see O'Connor, Stanford Technology Law Review. For an exemplary biometric system, see U.S. Pat. No. 6,119,096, Mann et al., which covers iris recognition for aircraft passenger check-in and boarding security. Large collections of SNPs, particularly the SNPs provided by the present invention, can be typed to uniquely identify an individual for biometric applications such as those described above. Such SNP typing can readily be accomplished using DNA chips as described above. Preferably, a minimally invasive means for obtaining a DNA sample is utilized. For example, PCR amplification enables sufficient DNA for analysis to be obtained from fingerprints, which contain DNA-containing skin cells and oils that are naturally transferred during contact.

### EXAMPLES

The following examples are intended to illustrate the present invention. The examples do not in any way limit the present invention to what is disclosed in the examples.

### Example 1

### Polymorphisms in the CEL VNTR and the Link to Exocrine Pancreatic Dysfunction and Diabetes

The link between polymorphisms in the CEL VNTR and exocrine pancreatic dysfunction and diabetes was first analyzed in two Families (Family 1 and Family 2) of North-European descent and were recruited from the Norwegian MODY Registry. In order to further explore the link between CEL VNTR variants and exocrine dysfunction, subjects were recruited consecutively among diabetic outpatients at Haukeland University Hospital between May 2004 and July 2005. Diabetic patients with FED were assigned as cases whereas diabetic patients without FED were assigned as controls. FED was defined as two fecal elastase samples <200 μg/g of which one sample had to be <100 μg/g. Of 247 patients, 182 patients were available for both genotyping and FED status determination. Of these 133 had been diagnosed with T1 D and 33 had been diagnosed with T2D by their physician, 16 had undetermined diabetes type.

### Family 1

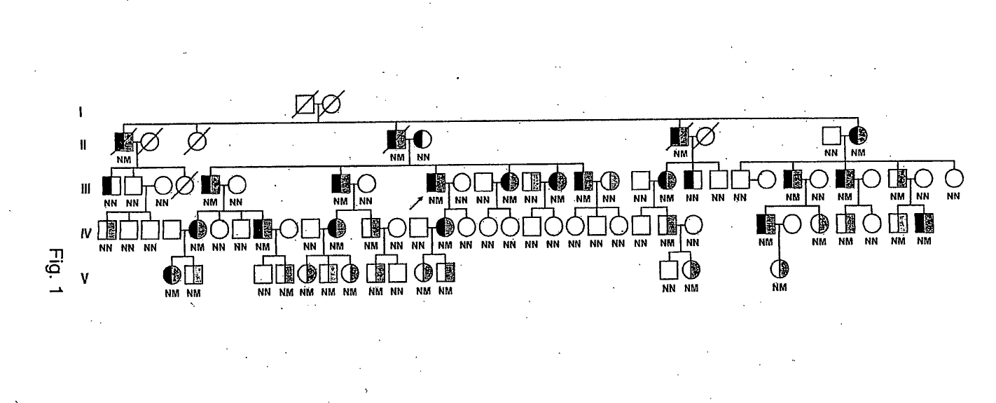

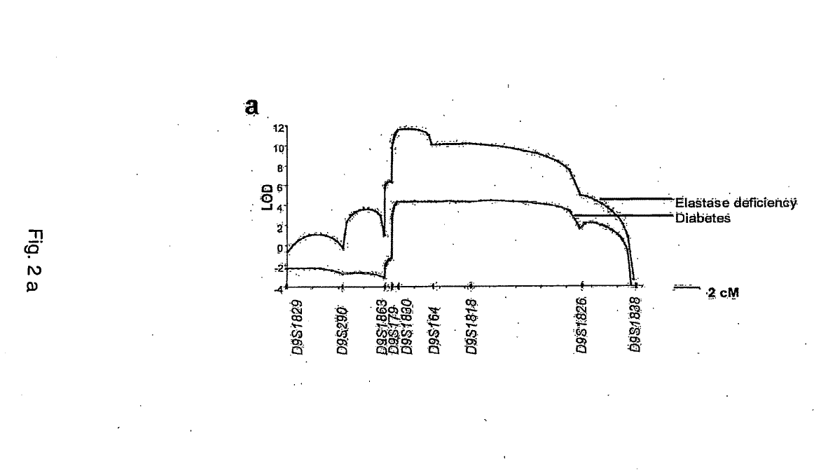

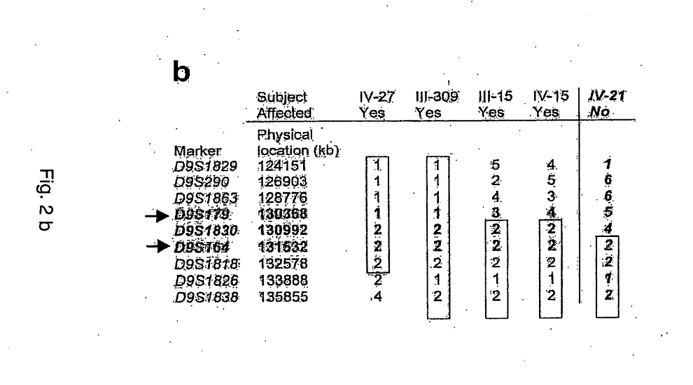

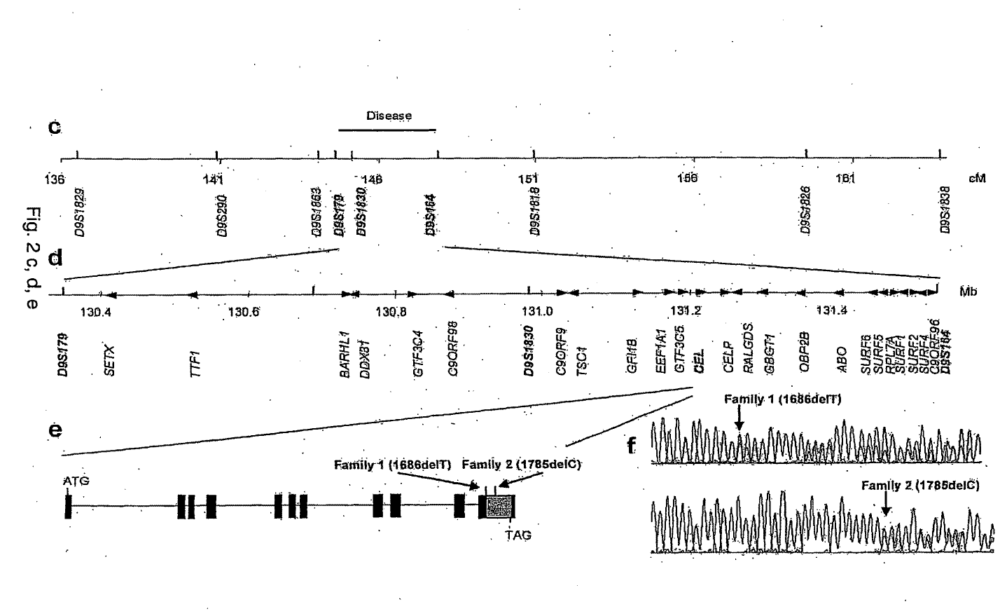

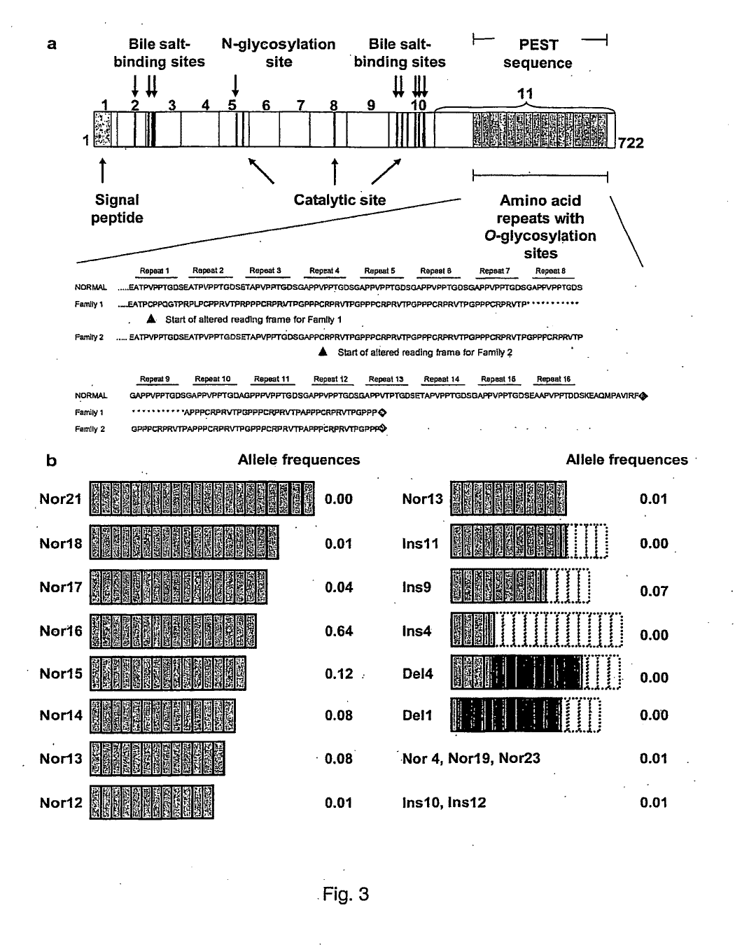

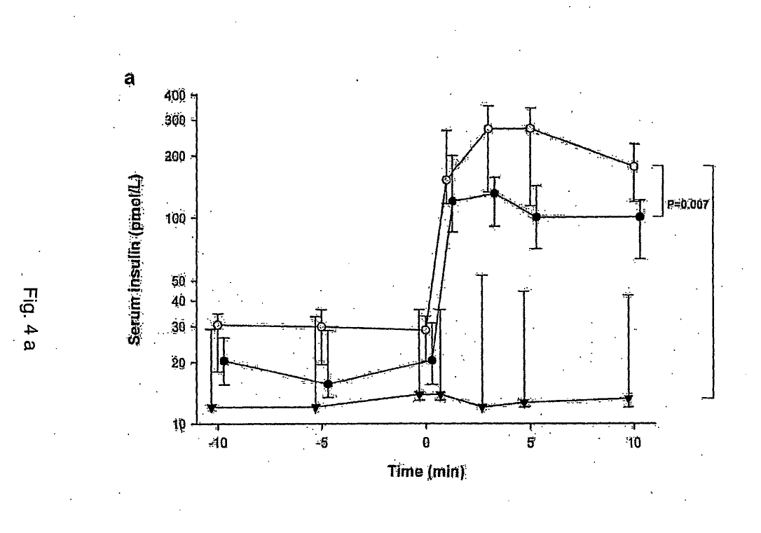

Genetic studies were carried out in a large family of 184 individuals with autosomal dominant diabetes typically detected before the age of forty and primary beta-cell dysfunction. These were genotyped for a dense set of microsatellite markers at chromosome 9q34. The maximum two-point and multipoint lod scores increased to 5.07 and 4.40, respectively, for diabetes and to 10.9 and 11.6 for FED as phenotype (Table 9; FIG. 2a). Haplotype analysis of recombinant chromosomes narrowed the critical region to 1.16 Mb of DNA flanked by the markers D9S179 and D9S164 (FIG. 2b). The candidate region contained 24 known genes (FIGS. 2c,d). Of these, only CEL, encoding carboxyl-ester lipase (also denoted bile salt-dependent lipase; OMIM #114840), was known to be both highly and predominantly expressed in the pancreas. Therefore the CEL gene was sequenced and a single-base deletion (1686delT; C563fsX673) in exon 11 (FIGS. 2e,f) was identified. The family's deletion occurred in the first repeat of a 14-repeat allele, and was predicted to alter the reading frame from amino acid residue 563 leading to a premature stop codon after residue 672 (FIG. 3a). The mutation co-segregated with diabetes and FED (FIG. 1) (maximum two-point lod scores of 4.47 and 11.6, respectively). Notably, the diabetic family member III-19, who did not carry the mutation, had normal elastase value suggesting diabetes from another cause (lod score increased from 4.47 to 5.94 if he was coded as unknown). The 1686delT mutation was not present in 370 control chromosomes.

### Clinical Studies of the Diabetes

Next the diabetes in Family 1 was characterized (Table 10; Table 6 shows the combined characteristics of Family 1 and Family 2).

TABLE 6
Clinical characteristics of patients in Family 1 and Family 2 with mutations in CEL.

| Characteristic | Diabetic | | NGT | | |
|---|---|---|---|---|---|
| [Normal range] | mutation carriers | P^{a} | mutation carriers | P^{a} | Controls^{b} |
| Number of subjects/males | 17/8 | | 17/11 | | 101/50 |
| Present age (yr) | 49 ± 12 | <0.001 | 15 ± 13 | 0.03 | 23 ± 16 |
| BMI (kg/m2) | 24 ± 2.9 | 0.12 | 19 ± 4.7 | 0.008 | 23 ± 5.2 |
| Endocrine pancreas dysfunction | | | | | |
| Mean age at diagnosis of | 34 ± 12 | | — | | — |
| diabetes (yr) | | | | | |
| HbA1C (%) [4.0-6.4] | 8.5 ± 1.4 | <0.001 | 5.5 ± 0.34 | 0.06 | 5.3 ± 0.29 |
| Fasting glucose (mmol/L) [3.9-6.0] | 10 ± 4.1 | <0.001 | 5.0 ± 0.49 | 0.48 | 5.2 ± 0.55 |
| Fasting C-peptide (nmol/L) | 0.25 ± 0.16 | <0.001 | 0.62 ± 0.38 | 0.81 | 0.60 ± 0.26 |
| [0.17-1.0] | | | | | |
| Exocrine pancreas dysfunction | | | | | |
| Fecal elastase-1 (μg/g) | 9.0 ± 22 | <0.001 | 62 ± 54 | <0.001 | 490 ± 90 |
| Fecal fat excretion (g/24 h)^{c} | 31 ± 15 | NT | 6.7 ± 0.58 | NT | NI |
| Vitamin A (μmol/L) [>0.70] | 1.4 ± 0.35 | 0.01 | 1.4 ± 0.44 | 0.03 | 1.7 ± 0.56 |
| Vitamin E (μmol/L) [>11.6] | 9.6 ± 6.0 | <0.001 | 15 ± 5.8 | 0.01 | 20 ± 6.4 |
| LDL cholesterol (mmol/L) [1.8-5.7] | 2.4 ± 0.65 | 0.02 | 2.4 ± 1.2 | 0.18 | 2.9 ± 0.76 |
| CT of pancreas^{d} | | | | | |
| volume (ml) | 39 ± 19 | <0.001 | 82 ± 28 | 0.29 | 84 ± 25 |
| X-ray attenuation (HU) | 19 ± 38 | 0.003 | −12 ± 22 | 0.0049 | 65 ± 28 |

Plus-minus values are means ± SD.
^{a}P-values were calculated by testing diabetic or NGT mutation carriers against controls.
^{b}Controls consisted of non-diabetic relatives (not spouses) without mutation.
^{c}Fecal fat excretion was measured in ten of the 17 diabetic mutation carriers and three of the 17 non-diabetic mutation carriers.
^{d}Here, the controls were age-and sex-matched to the diabetes mutation carriers and not related to the family.
NT, not tested;
NI, not investigated.

Of the 33 mutation carriers, 14 had diabetes, three had impaired glucose tolerance (IGT) (IV-8, IV-34, V-1) and eight had normal glucose tolerance (NGT; WHO criteria), while data from oral glucose tolerance testing (OGTT) were not available for eight non-diabetic mutation carriers. More than half of the diabetic mutation carriers were using insulin. No episodes of ketoacidosis had been recognized. Mean age at diagnosis was 36 years in Family 1. Thus, the large number of mutation carriers with NGT or IGT can be explained by the fact that most of them were considerably younger than mutation carriers with diabetes. Diabetic mutation carriers had normal body mass indices (BMI), moderately elevated levels of fasting glucose and 2-h glucose after an OGTT, clearly elevated glycosylated hemoglobins (HbA1c), reduced fasting C-peptides, and reduced insulin response during an intravenous glucose tolerance test (IVGTT; FIG. 4a; Table 10). Glucagon-stimulated C-peptide response also indicated decreased insulin secretory reserve (not shown). Taken together, these data indicate that the family exhibited some degree of beta-cell failure. IVGTT performed in NGT mutation carriers were suggestive of an early beta-cell dysfunction (FIG. 4a; Table 10). Fasting glucagon levels and proinsulin:insulin ratios were normal (not shown). No auto-antibodies against beta-cells, insulin, thyroid, adrenals or gluten/endomysium were detected. Three of the diabetic mutation carriers had microvascular complications. Among three deceased diabetic subjects with a confirmed disease haplotype (II-1, II-4 and II-6), two had had peripheral vascular disease with gangrene of the legs and the third subject had suffered from a cerebro-vascular infarction.

### Clinical Studies of the Exocrine Pancreatic Dysfunction

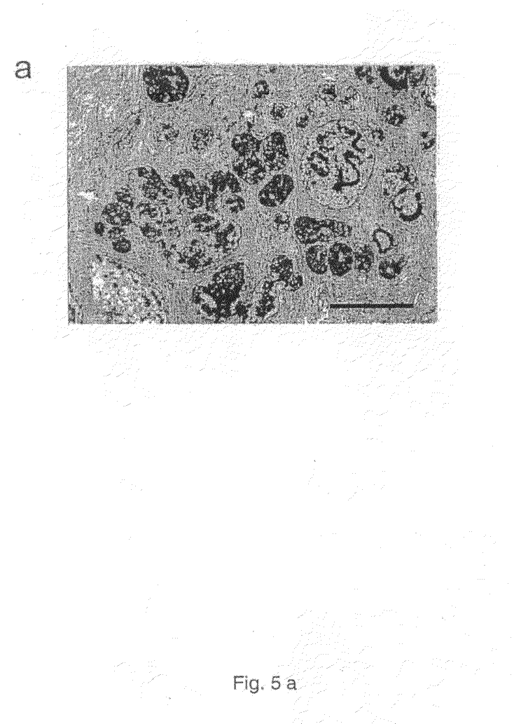

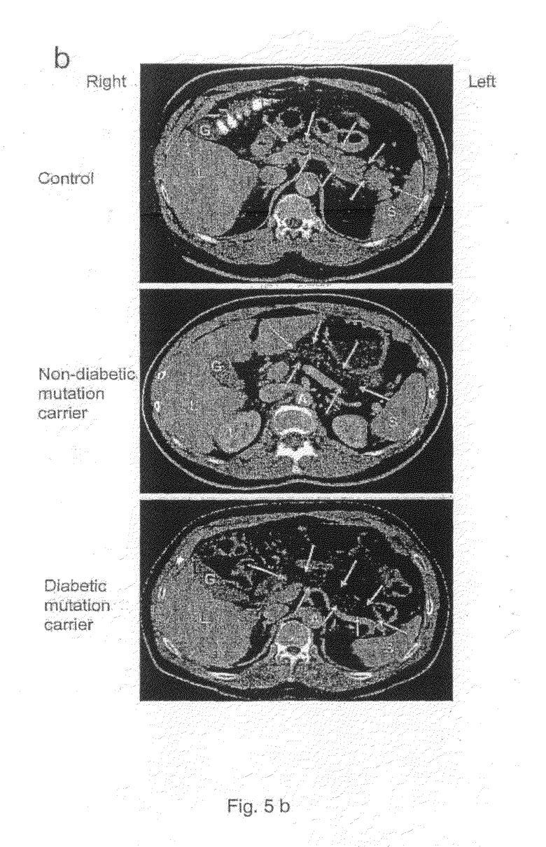

Mild abdominal symptoms and detection of FED (FIG. 4b) in Family 1 prompted to search more thoroughly for gastroenterological symptoms and signs. In retrospect, eight of 14 diabetic mutation carriers had experienced mild, recurrent abdominal pain, typically starting in the second decade of life, whereas diabetes had been diagnosed within the next 20 years. None of the living family members had diagnoses of pancreatitis or pancreatic cancer, or had undergone pancreatic surgery. However, chart reviews of the three deceased subjects with the disease haplotype revealed that steatorrhea and reduced bicarbonate secretion during secretin stimulation had been noted. Subject II-1, deceased at the age of 46, had been autopsied. The autopsy report described a macroscopically small and fibrotic pancreas. Histopathological examination of tissue sections revealed pronounced fibrosis and mucinous metaplasia (FIG. 5a). Islet or acinar cells could not be seen. Staining for chromogranin A, insulin and amyloid was negative (not shown). All tested mutation carriers had either severe (<100 □g/g, 30 individuals) or moderate (100-200 μg/g, three individuals) FED (FIG. 4b) suggesting exocrine pancreatic dysfunction. This was supported by the finding of increased levels of fecal fat excretion in all ten diabetic subjects tested and lowered levels of fat soluble vitamins in the mutation carriers (Table 10). Investigations revealed no signs compatible with inflammatory disease and specifically not with cystic fibrosis, inflammatory bowel disease or celiac disease. Furthermore, diabetic mutation carriers had reduced serum LDL cholesterol levels (Table 10). Computerized tomography (CT) of the pancreas in ten mutation carriers with diabetes and exocrine deficiency (FIGS. 5b, c; Table 10; Table 6) showed decreased pancreatic x-ray attenuation which was similar to that of visceral fat. The pancreatic volume adjusted for body surface area was significantly reduced. In four adult non-diabetic mutation carriers CT of the pancreas showed decreased pancreatic x-ray attenuation, while the pancreatic volume adjusted for body surface area was unchanged. Thus, abnormal pancreatic morphology was observed in all mutation carriers tested. Notably, there were no signs of pseudocysts, peripancreatic edema, calcifications or dilated ducts in any of the ten diabetic mutation carriers. Calcifications mainly in the pancreatic head were seen in one of the four NGT mutation carriers. None of them had dilated pancreatic ducts.

### Family 2

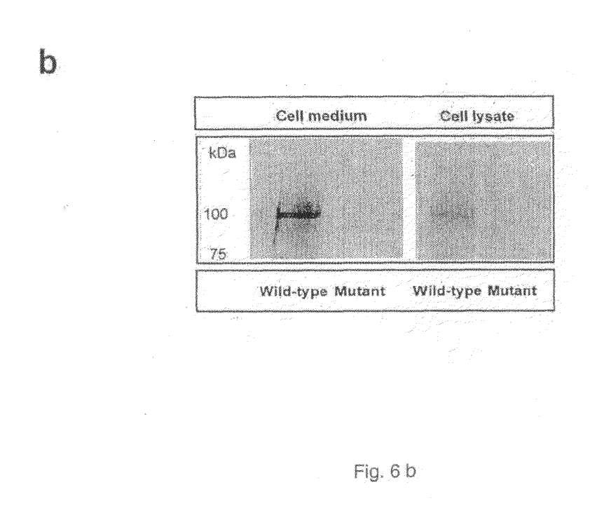

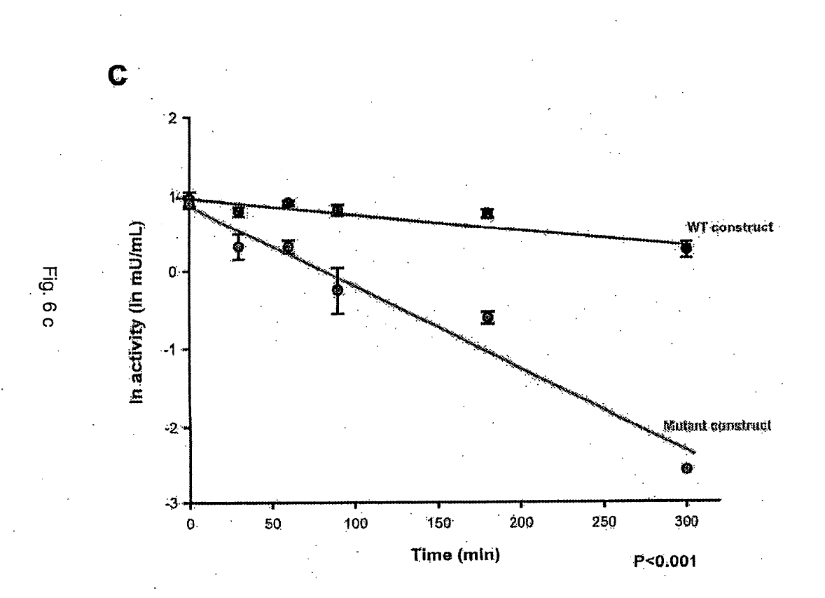

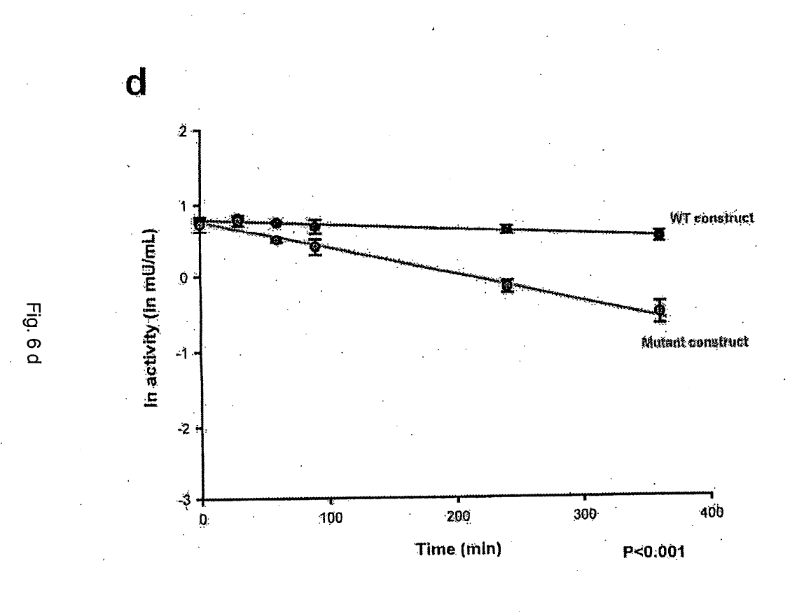

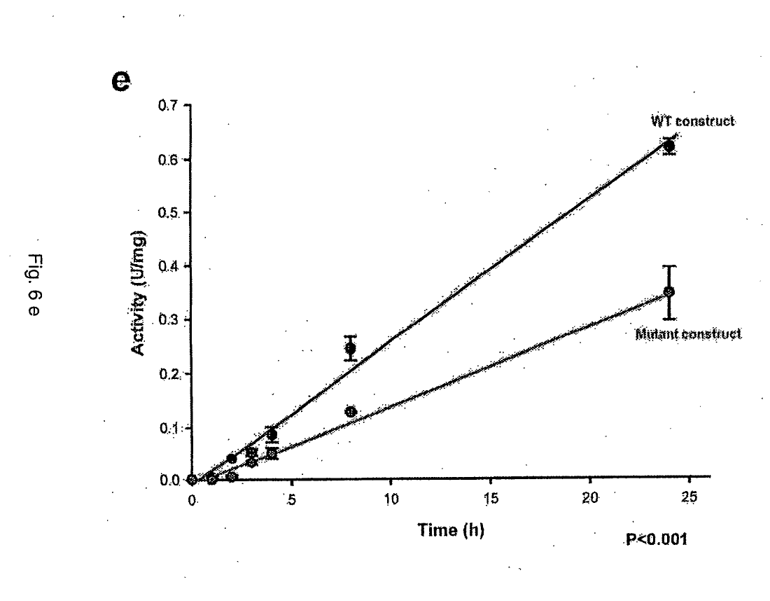

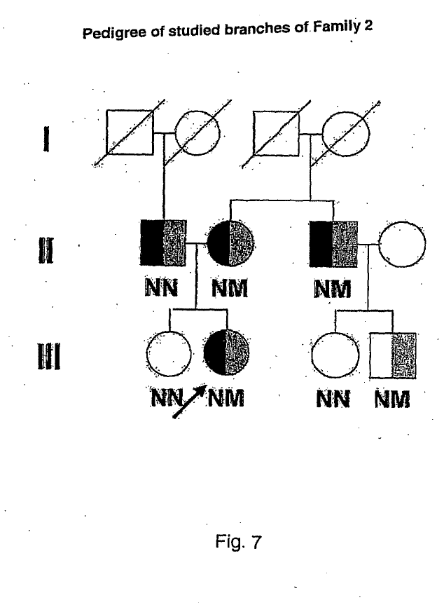

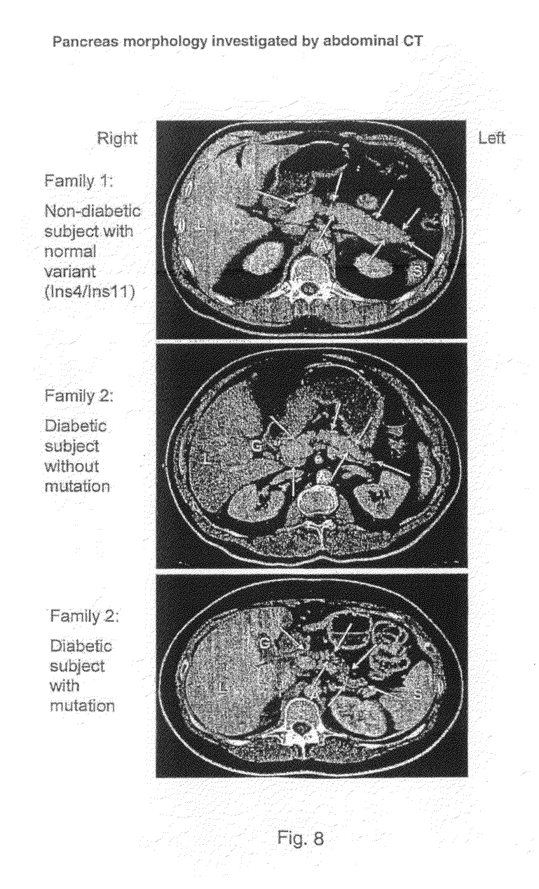

The CEL gene was sequenced in 38 probands from a Norwegian diabetes registry, known to be negative for disease-causing mutations in the MODY1-6 genes. One family (Family 2; FIG. 7) exhibited a single-base deletion (1785delC; C596fsX695) in the fourth repeating segment of exon 11, which was present in all at-risk family members with both diabetes and FED, or FED only (FIGS. 7 and 2e,f). The deletion-specific haplotype differed from that of the deletion-specific haplotype of Family 1 (not shown). Subject II-1 of Family 2 was not a true phenocopy since abdominal CT findings demonstrated normal pancreatic morphology (FIG. 8). Also an abdominal ultrasound showing hyper-echoic pancreas in the NGT mutation-carrying cousin of the proband (subject III-4 of Family 2; not shown) was performed. Apparently, structural changes of the pancreas could be used to exclude phenocopies, and this led to a lod score of 1.00 for FED in Family 2.

Due to the highly repetitive nature of exon 11, the frame-shift mutation of Family 2 was predicted to result in a protein containing a C-terminal region almost identical to that of Family 1 (FIG. 3b). The 1785delC mutation was not present in 370 control chromosomes. Further clinical investigations demonstrated that the endocrine and exocrine pancreatic dysfunction phenotypes of Family 2 were similar to that of Family 1 (Table 11).

Studies of Subjects with Common CEL Variants

Several other CEL allelic variants of exon 11 were identified in Families 1 and 2 (FIG. 3b). Most alleles differed in the number of the exon 11 segment repeats, but some contained single-base insertions within the VNTR, predicted to lead to premature truncation of the protein. The prevalence of the variants in 370 control chromosomes (FIG. 3b) was estimated. Family members with single-base insertions were tested for abnormal glucose tolerance and FED. The phenotype associated with the insertions was different from that associated with the two single-base deletions. Three of 32 subjects with insertions had a typical T2D without FED and one had FED without diabetes (IV-1 of Family 1). The latter subject carried one Ins4 and one Ins11 allele (FIG. 3b) and exhibited normal CT findings (FIG. 8). To further explore any link between common variants of CEL exon 11 and exocrine pancreatic dysfunction, 182 adult diabetic subjects with T1D or T2D were studied. Forty percent of the diabetic subjects with FED had single-base insertions in CEL compared to 14% of those without FED (OR=4.2 [1.6, 11.5]; Table 7).

The odds ratio remained significant for the subgroup of 133 T1D patients (OR=4.5 [1.4, 14.6]). In summary, the CEL gene is highly polymorphic, but only deletion alleles were associated with monogenic disease and absent in blood donors. The insertion alleles, which together occur at a frequency of 0.08, may, however, be associated with an increased risk of exocrine dysfunction in diabetic patients.

TABLE 7
Subjects with T1D or T2D grouped according to FED status
and CEL insertion status.

| | No insertion | Insertion^{a} | |
|---|---|---|---|
| No FED | 140 (86%) | 22 (14%) | P = 0.007 |
| FED | 12 (60%) | 8 (40%) | |

The number of diabetic outpatients in each group is given. Of all subjects, 11% (20/182) had FED.
^{a}Insertion denotes the presence of a single-base insertion in the CEL exon 11 VNTR, independent of repeat position. The position of the single-base insertions varied, being located either in repeat 9, 10, 11 or 12. Twenty-nine subjects were heterozygous and one was homozygous for single-base insertions.

### Protein and RNA Studies

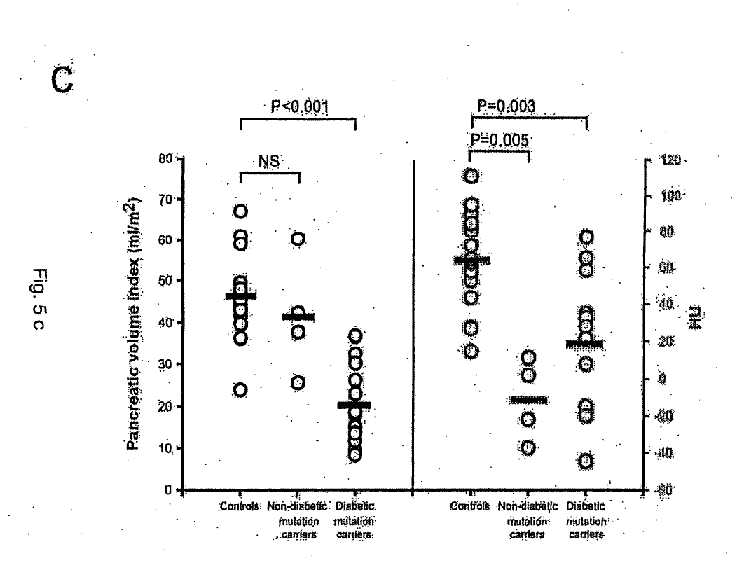

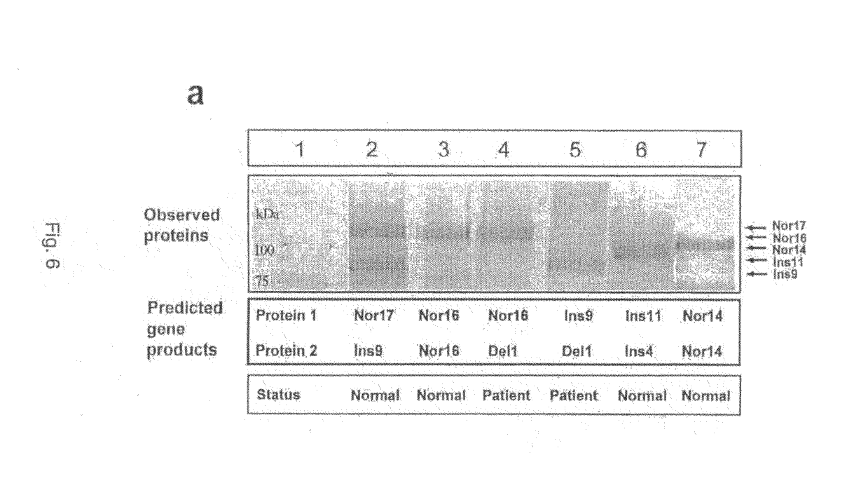

Because the CEL protein can be detected in urine of healthy persons (unpublished observations), urine from family members was examined with Western blotting using the CEL-specific antibody pAbL64 (FIG. 6a). In control subjects with normal variants, bands corresponding to the predicted number of repetitive segments in axon 11 were recognized. In disease-associated mutation carriers, only the band corresponding to the normal allele could be detected. Thus, any protein arising from the disease allele was either not recognized by the antibody or absent in patient urine samples. No band was, however, visible using the pAbL64 antibody on Western Blots of Chinese hamster ovary (CHO) cell lines expressing the mutant protein (FIG. 6b) although CEL enzyme activity was recorded in the medium of these cell lines (FIGS. 6c,d,e). Therefore, mutant protein is most likely not recognized by the antibody. The wild-type band (FIG. 6b) appeared shorter in the cell line experiments compared to urine, probably because of different glycosylation patterns in CHO cells and urine.

The Vₘₐₓ and Kₘ of the enzyme was assessed using recombinant normal and mutant protein with 4-nitrophenyl hexanoate (4-NH) and cholesteryl oleate (CO) as substrates. The catalytic efficiencies (i.e. the ratio Vₘₐₓ/Kₘ) of wild-type and mutant (1686delT; Family 1) CEL were similar (1.5 10⁻² versus 1.1 10⁻² min⁻¹, respectively) when recorded with the water soluble substrate 4-NH. Comparable catalytic efficiencies were found using micellar CO as substrate (2.1 10² versus 0.9 10⁻² min⁻¹ for wild-type and mutant recombinant enzyme, respectively). Since the catalytic properties of the mutant enzyme were not severely affected, protein stability and rate of secretion in culture media of stably transfected CHO cells were studied. Mutant CEL was less stable both at 37° C. and 4° C. (FIGS. 6c,d). Moreover, the rate of secretion of the mutant enzyme was significantly reduced (FIG. 6e). A control experiment performed by quantitative real-time PCR demonstrated that the wild-type and mutant transfected CHO cell lines expressed equal amounts of CEL mRNA (data not shown).

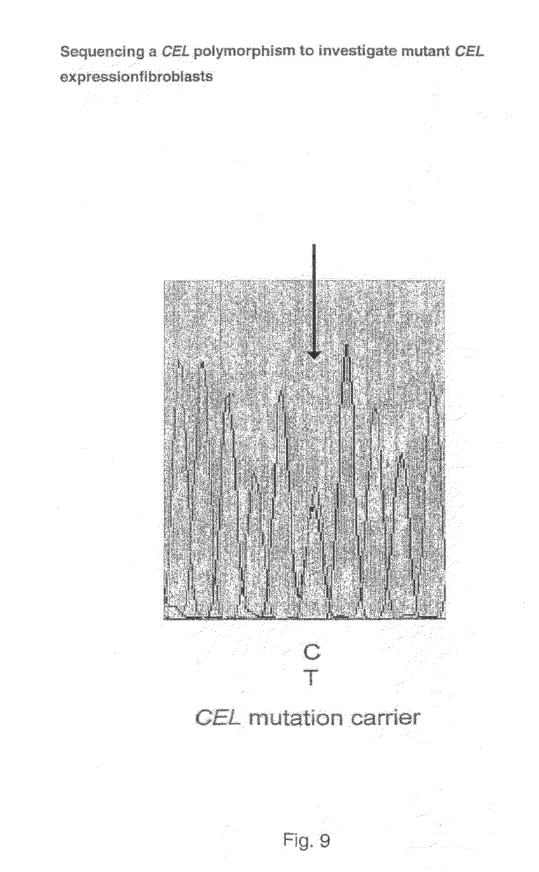

To investigate whether the expression level of the mutated allele could be affected in the patients, mRNA was isolated from skin fibroblasts of a mutation carrier. By RT-PCR and sequencing, it was found that the normal and mutated CEL alleles appeared to be expressed in equal amounts (FIG. 9).

### Example 2

### Simple Method for the Detection of Insertions or Deletions in the First Six Repeats of the CEL VNTR in Exon 11

### Overview of Method

The presented assay provides a simple way to test for insertion or deletion variation in the first six repeats of the CEL VNTR in exon 11. The number of CEL VNTR repeats is also determined. The method is based on duplex PCR using fluorescent primers followed by fragment analysis on a high-resolution capillary machine, for example the ABI-3730 sequencer. Insertions/deletions will be detected by a shift in migration length.

### Primers for the PCR Reaction

A duplex PCR with
1. one fwd-primer located at the beginning of the CEL-VNTR (pink): primer1: 5′-ACC GAC CAG GAG GCC ACC C-3′ (SEQ ID NO: 26)
2. One rev-primer binding to all repeats harbouring the exact sequences GTGACTCCGGGGCC (SEQ ID NO:29) (FAM-blue) primer2-FAM: 5′-TAC TCG AGG GTGGCC CCG GAG TCA C-3′ (SEQ ID NO: 27)
3. Another rev-primer binding specifically to the 3′ end of the gene (NED-green): Primer3-NED: 5′-CCT GGG GTC CCA CTC TTG T-3′ (SEQ ID NO:28)
   Products from the PCR Reaction

Products obtained from duplex PCR reactions using the above-mentioned primers 1, 2 and 3 were as follows:

Primers 1 and 3 give one peak for each repeat allele (for example two peaks for a heterozygous individual with repeat length 14/16)

Primers 1 and 2 give one peak for each repeat carrying the sequence GTGACTCCGGGGCC (SEQ ID NO: 29) (for each allele)

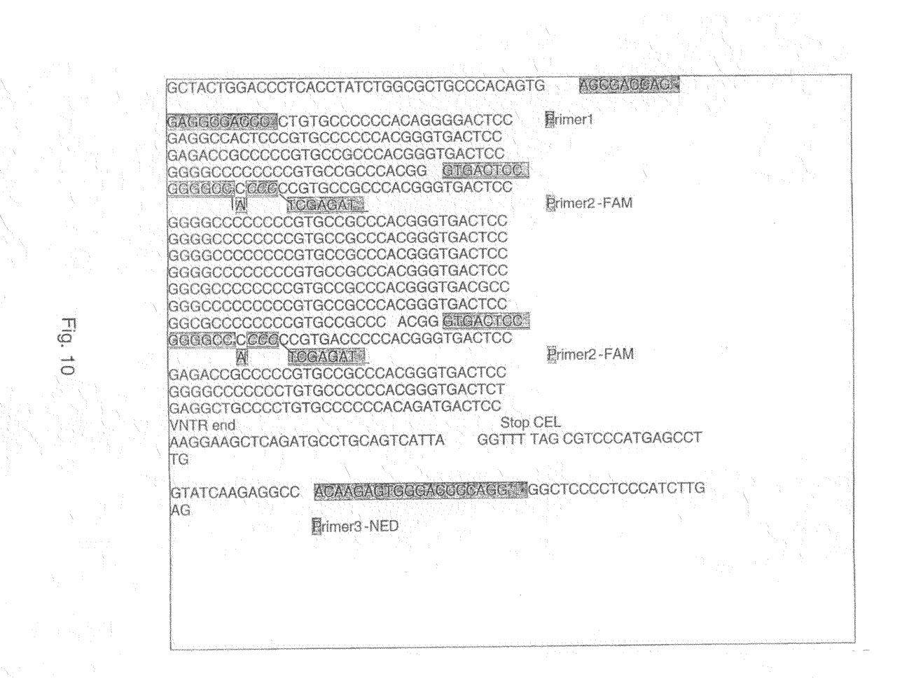

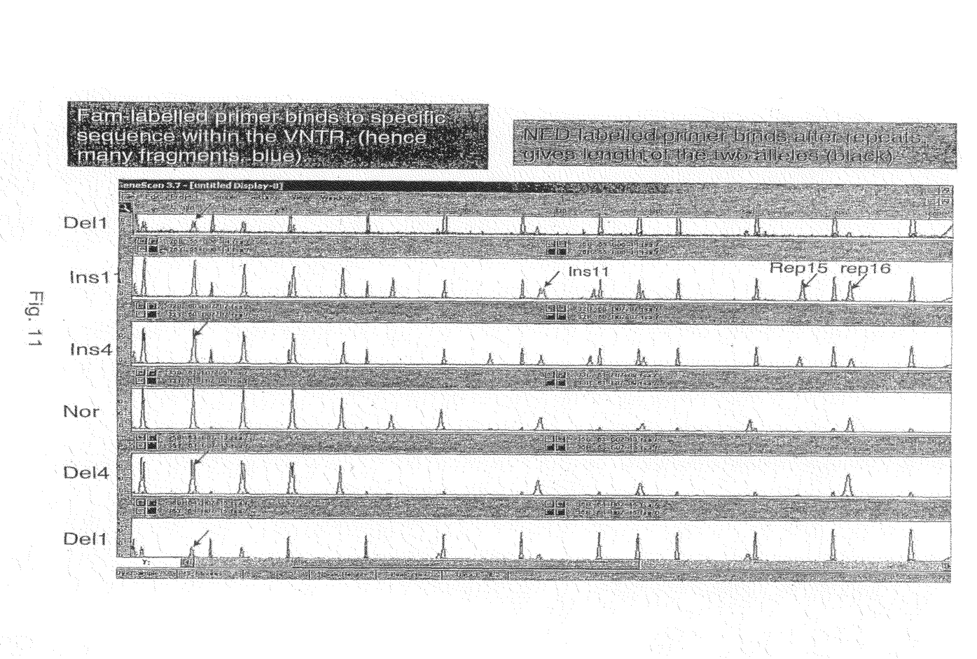

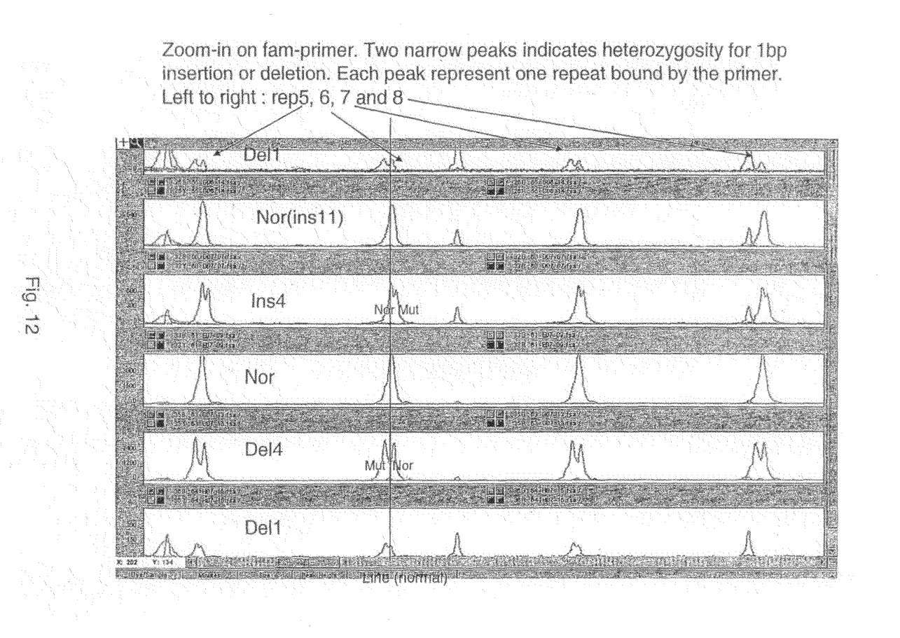

Reference is made to FIGS. 10, 11 and 12. FIG. 10 illustrates the location of the primers and FIG. 11 illustrates the various patterns obtained by using the fragment analyzer software from ABI. FIG. 12 is a “zoom in”-illustration of the obtained peaks which indicate heterozygosity for 1 bp insertion or deletion.

### Fragment Analysis Protocol

### VNTR Length and Detection of Insertions and Deletions

Based on the TaKaRa LaTaq with GC-Buffer Kit.

| | No. of |
|---|---|
| 8 ul reaction | reactions |
| X1 | 100 |

| | | |
|---|---|---|
| PCR: | | |
| 2XGC-buffer 1 | 4,42 | 441,88 |
| dNTP-mix (2,5 mM each) | 1,40 | 140,00 |
| Primer1 (20 uM) | 0,26 | 26,25 |
| Primer3-NED (20 uM) | 0,26 | 26,25 |
| Primer2-FAM | 0,05 | 5,25 |
| Betain (5M) | 2,01 | 201,25 |
| LaTaq (5U/ul) | 0,04 | 4,38 |
| Total | 8,5 | 845,25 |
| PCR-reaction: | | |

| | |
|---|---|
| 94 | 1 min |
| 94 | 30 sek |
| 61 | 30 sek |
| 72 | 1 min |
| 94 | 20 sek |
| 72 | 5 min |
| 4 | ∞ |

8 ul mm per reaction
Template: 2 ul DNA
Primer1: 5′-ACC GAC CAG GAG GCC ACC C-3′
(SEQ ID NO: 26)
Primer2-FAM 5′-TAC TCG AGG GTGGCC CCG GAG TCA C-3′
(SEQ ID NO: 27)
Primer3-NED 5′-CCT GGG GTC CCA CTC TTG T-3′
(SEQ ID NO: 28)

Fragment Analysis (FA):

Use 1 ul template from above
Pipette into 9 ul of FA-mix

$$\begin{matrix}\frac{FA \text{-} mix}{Formamide} & \frac{X 100}{980 ul} \\ X \text{-} Rhodamine MapMarker 1000 & 20 ul\end{matrix} \} 95 ° C . 2 min , put on ice$$

Run on ABI3100, filter set D

[symbol]

### REFERENCES

1. Henderson, J. R., Why are the islets of Langerhans? Lancet, 1969. 2 (7618): p. 469-70.
2. Cavalot, F., et al., Pancreatic elastase-1 in stools, a marker of exocrine pancreas function, correlates with both residual beta-cell secretion and metabolic control in type 1 diabetic subjects. Diabetes Care, 2004. 27 (8): p. 2052-4.
3. Lombardo, F., et al., Natural history of glucose tolerance, beta-cell function and peripheral insulin sensitivity in cystic fibrosis patients with fasting euglycemia. Eur J Endocrinol, 2003.149 (1): p. 53-9.
4. Mitchell, R. M., M. F. Byrne, and J. Baillie, Pancreatitis. Lancet, 2003. 361 (9367): p. 1447-55.
5. Wang, F., et al., The relationship between diabetes and pancreatic cancer. Mol Cancer, 2003. 2: p. 4.
6. Bellanne-Chantelot, C., et al., Clinical spectrum associated with hepatocyte nuclear factor-1beta mutations. Ann Intern Med, 2004. 140 (7): p. 510-7.
7. Haumaitre, C., et al., Lack of TCF2/vHNF1 in mice leads to pancreas agenesis. Proc Natl Acad Sci USA, 2005. 102 (5): p. 1490-5.
8. Sellick, G. S., et al., Mutations in PTF1A cause pancreatic and cerebellar agenesis. Nat Genet, 2004. 36 (12): p. 1301-5.
9. Stoffers, D. A., et al., Pancreatic agenesis attributable to a single nucleotide deletion in the human IPF1 gene coding sequence. Nat Genet, 1997. 15 (1): p. 106-10.
10. Edlund, H., Pancreatic organogenesis-developmental mechanisms and implications for therapy. Nat Rev Genet, 2002. 3 (7): p. 524-32.
11. Lindquist, S., L. Blackberg, and O. Hernell, Human bile salt-stimulated lipase has a high frequency of size variation due to a hypervariable region in exon 11. Eur J Biochem, 2002. 269 (3): p. 759-67.
12. Stromqvist, M., et al., Naturally occurring variants of human milk bile salt-stimulated lipase. Arch Biochem Biophys, 1997. 347 (1): p. 30-6.
13. Taylor, A. K., et al., Carboxyl ester lipase: a highly polymorphic locus on human chromosome 9qter. Genomics, 1991. 10 (2): p. 425-31.
14. Miyasaka, K., et al., Carboxylester lipase gene polymorphism as a risk of alcohol-induced pancreatitis. Pancreas, 2005. 30 (4): p. e87-91.
15. Bengtsson-Ellmark, S. H., et al., Association between a polymorphism in the carboxyl ester lipase gene and serum cholesterol profile. Eur J Hum Genet, 2004.12 (8): p. 627-32.
16. Hui, D. Y., Molecular biology of enzymes involved with cholesterol ester hydrolysis in mammalian tissues. Biochim Biophys Acta, 1996.1303 (1): p. 1-14.
17. Rudd, E. A., and H. L. Brockmann, Pancreatic carboxyl ester lipase (cholesterol esteras). In Lipases B. Borgstrom and H. L. Brockmann, editors. 1984, New York: Elsevier Science. 185-204.
18. Wang, C. S. and J. A. Hartsuck, Bile salt-activated lipase. A multiple function lipolytic enzyme. Biochim Biophys Acta, 1993. 1166 (1): p. 1-19.
19. Hui, D. Y. and P. N. Howles, Carboxyl ester lipase: structure-function relationship and physiological role in lipoprotein metabolism and atherosclerosis. J Lipid Res, 2002. 43 (12): p. 2017-30.
20. Hardt, P. D., et al., High prevalence of exocrine pancreatic insufficiency in diabetes mellitus. A multicenter study screening fecal elastase 1 concentrations in 1,021 diabetic patients. Pancreatology, 2003. 3 (5): p. 395-402.

### Description of the Gene Sequences of the Invention

In the following DNA sequences a coding sequence is indicated by capital letters and non-coding sequence by small cases.

SEQ ID NO:1
CEL geonmic sequence
>hg17_knownGene_NM_001807 range = chr9:
132966919-132976801 5′pad = 0
3′pad = 0 revComp = FALSE strand = +
repeatMasking = none
GGCCACCCAGAGGCTGATGCTCACCATGGGGCGCCTGCAACTGGTTGTGT
TGGGCCTCACCTGCTGCTGGGCAGTGGCGAGTGCCGCGAAGgtaagagcc
cagcagaggggcaggtcctgctgctctctcgctcaatcagatctggaaac
ttcgggccaggctgagaaagagcccagcacagccccgcagcagatcccgg
gcactcacgctcatttctatggggacaggtgccaggtagaacacaggatg
cccaattccatttgaatttcagataaactgccaagaactgctgtgtaagt
atgtcccatgcaatatttgaaacaaatttctatgggccgggcgcagtggc
tcacacctgcaatcccaccagtttgggaggccgaggtgggtggatcactt
gaggtcaggagttggagaccagcctggccaacatggtgaaaccccgtctc
tactaaaaatacaaatattaatcgggcgtggtggtgggtgcctgtaatcc
cagctactcgggaggctgaggcaggagaaccgcttgaagctgggaggtgg
agattgcggtgagctgagatcacgctactgcactccagcctgggtgacag
ggcgagactctgtctcaaaaaatagaaaaagaaaaaaatgaaacatacta
aaaaacaattcactgtttacctgaaattcaaatgtaactgggcctcttga
atttacatttgctaatcctggtgattccacctctctgttgttcccatttt
acagaaggggaaacgggcccaggggcagggagtgtggagagcaggcagac
gggtggagagaagcaggcaggcagtttgcccagcatggcacagctgctgc
ctcctattcctgtgcaggaagctgaaagccgagctactccacacccgggt
ccgggtccctccagaaagagagccggcaggcaggagctctctcgaggcat
ccataaattctaccctctctgcctgtgaaggagaagccacagaaacccca
agccccacaggaagccggtgtcggtgcccggcccagtccctgcccccagc
aggagtcacacaggggaccccagatcccaaccacgctgttctgccgcctg
cggtgtctcaggccctggggactcctgtctccacctctgctgcctgctct
ccacactccctggccctgggaccgggaggtttgggcagtggtcctgggct
cctgactcaaaggagaggtcaccttcttcttgggcgagctcttcttgggg
tgctgagaggccttcggcaggtcatcacgacccctccccatttccccacc
ctgaggccctctggccagtctcaattgcacagggatcacgccactggcac
aaggagacacagatgcctcgcaggggatgcccacgatgcctgcatgtgtt
gcttctggttcctttcctccagttccaaccgccgcactctcccacaccag
tgtgacagggggcccatcaccctagacttcagagggctgctgggaccctg
gctgggcctgggggtgtagggccaccctgcccttccccacctggaacctg
gcacaggtgacagccagcaagcaatgacctggtcccaccatgcaccacgg
gaagagggagctgctgcccaagatggacaggaggtggcactggggcagac
agctgcttctcaacagggtgacttcaagcccaaaagctgcccagcctcag
ttccgtcagggacagagggtggatgagcaccaacctccaggcccctcgtg
ggggtggacagcttggtgcacagaggccattttcatggcacagggaagcg
tggcgggggtgggaggtgtggtccctagggggttctttaccagcaggggg
ctcaggaactgtggggacttgggcatggggccatcgactttgtgcccagc
cagctaggccctgtgcagggagatgggaggagggaaaagcaggccccacc
cctcagaaaggaggaaggttggtgtgaaacatcccgggtacactgagcat
tgggtacactcctcccgggagctggacaggcctcccatgtgatggcaaac
aggccgacaggagacacggctgttgctcgtcttccacatggggaaactga
ggatcggagtcaaagctgggcggccatagccagaacccaaacctccatcc
cacctcttggccggcttccctagtgggaacactggttgaaccagtttcct
ctaagattctgggagcaggacacccccagggataaggagaggaacaggaa
tcctaaagccctgagcattgcagggcagggggtgctgcctgggtctcctg
tgcagagctgtcctgctttgaagctgtctttgcctctgggcacgcggagt
cggcttgccttgccccctccggattcaggccgatggggcttgagcccccc
tgaccctgcccgtgtctccctcgcagCTGGGCGCCGTGTACACAGAAGGT
GGGTTCGTGGAAGGCGTCAATAAGAAGCTCGGCCTCCTGGGTGACTCTGT
GGACATCTTCAAGGGCATCCCCTTCGCAGCTCCCACCAAGGCCCTGGAAA
ATCCTCAGCCACATCCTGGCTGGCAAGgtgggagtgggtggtgccggact
ggccctgcggcggggcgggtgagggcggctgccttcctcatgccaactcc
tgccacctgcagGGACCCTGAAGGCCAAGAACTTCAAGAAGAGATGCCTG
CAGGCCACCATCACCCAGGACAGCACCTACGGGGATGAAGACTGCCTGTA
CCTCAACATTTGGGTGCCCCAGGGCAGGAAGCAAGgtctgcctcccctct
actccccaagggaccctcccatgcagccactgccccgggtctactcctgg
cttgagtctgggggctgcaaagctgaacttccatgaaatcccacagaggc
ggggaggggagcgcccactgccgttgcccagcctggggcagggcagcgcc
ttggagcacctccctgtcttggccccaggcacctgctgcacagggacagg
ggaccggctggagacagggccaggcggggcgtctggggtcaccagccgct
cccccatctcagTCTCCCGGGACCTGCCCGTTATGATCTGGATCTATGGA
GGCGCCTTCCTCATGGGGTCCGGCCATGGGGCCAACTTCCTCAACAACTA
CCTGTATGACGGCGAGGAGATCGCCACACGCGGAAACGTCATCGTGGTCA
CCTTCAACTACCGTGTCGGCCCCCTTGGGTTCCTCAGCACTGGGGACGCC
AATCTGCCAGgtgcgtgggtgccttcggccctgaggtggggcgaccagca
tgctgagcccagcagggagattttcctcagcacccctcaccccaaacaac
cagtggcggttcacagaaagacccggaagctggagtagaatcatgagatg
caggaggcccttggtagctgtagtaaaataaaagatgctgcagaggccgg
gagagatggctcacgcctgtaatcccagcactttaggaggcccacacagg
tgggtcacttgagcgcagaagttcaagaccagcctgaaaatcactgggag
acccccatctctacacaaaaattaaaaattagctggggactgggcgcggc
ggctcacccctgtaatcccagcacgttgggagcccaaggtgggtagatca
cctgaggtcaggagtttgagaccagcctgactaaaatggagaaacctctt
ctctactaaaaatacaaaattagccaggcgtggtggcgcttgcctgtaat
cccagctactcgggaggctgaggcaggagaatcgcttgaactcaggaggc
ggaggttgcggtgagccgagatcatgccactgcactccagcctggagaac
aagagtaaaactctgtctcaaaaaaaaaaaaaaaaaaaaaaatagccagg
cgtggtatctcatgcctctgtcctcagctacctgggaggcagaggtggaa
ggatcgcttgagcccaggggttcaaagctgcagtgagccgtggtcgtgcc
actgcactccagcctgggcgacagagtgaggccccatctcaaaaataaga
ggctgtgggacagacagacaggcagacaggctgaggctcagagagaaacc
aggagagcagagctgagtgagagacagagaacaataccttgaggcagaga
cagctgtggacacagaagtggcaggacacagacaggagggactggggcag
gggcaggagaggtgcatgggcctgaccatcctgcccccgacaaacaccac
cccctccagcaccacaccaacccaacctcctggggacccaccccatacag
caccgcacccgactcagcctcctggggacccacccactccagcaaccaac
gtgacctagtctcctggggacccaccccctccagcaccctacccgaccca
gcttcttagggacccaccatttgccaactggggctctgccatggccccaa
ctctgttgagggcatttccaccccacctatgctgatctcccctcctggag
gccaggcctgggccactggtctctagcaccccctcccctgccctgccccc
agGTAACTATGGCCTTCGGGATCAGCACATGGCCATTGCTTGGGTGAAGA
GGAATATCGCGGCCTTCGGGGGGGACCCCAACAACATCACGCTCTTCGGG
GAGTCTGCTGGAGGTGCCAGCGTCTCTCTGCAGgtctcgggatccctgtg
gggagggcctgccccacaggttgagaggaagctcaaacgggaaggggagg
gtgggaggaggagcgtggagctggggctgtggtgctggggtgtccttgtc
ccagcgtggggtgggcagagtggggagcggccttggtgacgggatttctg
ggtcccgtagACCCTCTCCCCCTACAACAAGGGCCTCATCCGGCGAGCCA
TCAGCCAGAGCGGCGTGGCCCTGAGTCCCTGGGTCATCCAGAAAAACCCA
CTCTTcTGGGCCAAAAAGgtaaacggaggagggcagggctgggcggggtg
ggggctgtccacatttccgttctttatcctggaccccatccttgccttca
aatggttctgagccctgagctccggcctcacctacctgctggccttggtt
ctgcccccagGTGGCTGAGAAGGTGGGTTGCCCTGTGGGTGATGCCGCCA
GGATGGCCCAGTGTCTGAAGGTTACTGATCCCCGAGCCCTGACGCTGGCC
TATAAGGTGCCGCTGGCAGGCCTGGAGTgtgagtagctgctcgggttggc
ccatggggtctcgaggtgggggttgaggggggtactgccagggagtactc
cggaggagagaggaaggtgccagagctgcggtcttgtcctgtcaccaact
agctggtgtctcccctcgaaggccccagctgtaagggagagggggtgccg
tttcttctttttttttgagatggagtctcactgttgcccaggctggagtg
cagtgtcacgatctcagctcactgcaacctccacctcctgggttcaagtg
attctctgactcaacctcccatgtagctgggactacaggcacatgccacc
atgcccagataatttttctgtgtgtttagtagggatggagtttcatcgtg
ttagctaggatgatctcggtcttgggacctcatgatctgcccacctcggc
ctcccaaagtgctggaattacaggcgtgagccactgtgcccggccccttc
tttattcttatctcccatgagttacagactcccctttgagaagctgatga
acatttggggccccctcccccacctcatgcattcatatgcagtcatttgc
atataattttagggagactcatagacctcagaccaagagcctttgtgcta
gatgaccgttcattcattcgttcattcattcagcaaacatttactgaacc
gtagcactggggcccagcctccagctccactattctgtaccccgggaagg
cctggggacccattccacaaacacctctgcatgtcagccttaccagcttg
ctacgctaaggctgtccctcactcattcttctatggcaacatgccatgaa
gccaagtcatctgcacgtttacctgacatgagctcaactgcacgggctgg
acaagcccaaacaaagcaacccccacggccccgctagaagcaaaacctgc
tgtgctgggcccagtgacagccaggccccgcctgcctcagcagccactgg
gtcctctaggggcccgtccaggggtctggagtacaatgcagacctcccac
catttttggctgatggactggaacccagccctgagagagggagctccttc
tccatcagttccctcagtggcttctaagtttcctccttcctgcttcaggc
ccagcaaagagagagaggagagggaggggctgccgctgaagaggacagat
ctggccctagacagtgactctcagcctggggacgtgtggcagggcctgga
gacatctgtgattgtcacagctggggagggggtgctcctggcacctcgtg
ggtcgaggccggggatgctctaaacatcctacagggcacaggatgcccct
gatggtgcagaatcaaccctgccccaagtgtccatagatcagagaaggga
ggacatagccaattccagccctgagaggcaaggggcggctcaggggaaac
tgggaggtacaagaacctgctaacctgctggctctcccacccagACCCCA
TGCTGCACTATGTGGGCTTCGTCCCTGTCATTGATGGAGACTTCATCCCC
GCTGACCCGATCAACCTGTACGCCAACGCCGCCGACATCGACTATATAGC
AGGCACCAACAACATGGACGGCCACATCTTCGCCAGCATCGACATGCCTG
CCATCAACAAGGGCAACAAGAAAGTCACGGAgtaagcagggggcacagga
ctcaggggcgacccgtgcgggagggccgccgggaaagcactggcgagggg
gccagcctggaggaggaaggcattgagtggaggactgggagtgaggaagt
tagcaccggtcggggtgagtatgcacacaccttcctgttggcacaggctg
agtgtcagtgcctacttgattcccccagGGAGGACTTCTAcAAGCTGGTC
AGTGAGTTCACAATCACCAAGGGGCTCAGAGGCGCCAAGACGACCTTTGA
TGTCTACACCGAGTCCTGGGCCCAGGACCCATCCCAGGAGAATAAGAAGA
AGACTGTGGTGGACTTTGAGACCGATGTCCTCTTCCTGGTGCCCACCGAG
ATTGCCCTAGCCCAGCACAGAGCCAATGCCAAgtgaggatctgggcagcg
ggtggctcctgggggccttcctggggtgctgcaccttccagccgaggcct
cgctgtgggtggctctcaggtgtctgggttgtctgggaaagtggtgcttg
agtccccacctgtgcctgcctgatccactttgctgaggcctggcaagact
tgagggcctctttttacctcccagcctacagggctttacaaaccctatga
tcctctgccctgctcagccctgcaccccatggtccttcccactggagagt
tcttgagctaccttccatcccccatgctgtgtgcactgagagaacactgg
acaatagtttctatccactgactcttatgggcctcaactttgcccataat
ttcagcccaccaccacattaaaaatcttcatgtaataatagccaattata
ataaaaaataaggccagacacagtagctcatgcctgtaatcccagcacat
tgggaggtcaaggtgggaggatcacttgaggtcaggagtctgagactagt
ctggccaacatggcaaaaccccatctctactaaaaatacaaaaattatcc
aggcatggtggtgcatgcctataatcctagctactcaggaggctgaggta
gcagaattgattgacccagggaggtggaggttgcagtgagccgagattac
gccactgcactccagcaggggcaacagagtgagactgtgtctcgaataaa
taagtaaataaataataaaaataaaaaataagttaggaatacgaaaaaga
taggaagataaaagtatacctagaagtctaggatgaaagctttgcagcaa
ctaagcagtacatttagctgtgagcctcctttcagtcaaggcaaaaaggg
aaacagttgagggcctataccttgtccaatctaattgaagaatgcacatt
cacttggagagcaaaatatttcttgatactgaattctagaaggaaggtgc
ctcacaatgttttgtggaggtgaagtataaattcagctgaaattgtggaa
cccatgaatccatgaatttggttctcagctttcccttccctgggtgtaag
aagccccatctcttcatgtgaattccccagacacttccctgcccactgcc
cgggacctccctccaagtccggtctctgggctgatcggtccccagtgagc
accctgcctacttgggtggtctctcccctccagGAGTGCCAAGACCTACG
CCTACCTGTTTTCCCATCCCTCTCGGATGCCCGTCTACCCCAAATGGGTG
GGGGCCGACCATGCAGATGACATTCAGTACGTTTTCGGGAAGCCCTTCGC
CACCCCCACGGGCTACCGGCCCCAAGACAGGACAGTCTCTAAGGCCATGA
TCGCCTAcTGGACCAACTTTGCCAAAACAGGgtaagacgtgggttgagtg
cagggcggagggccacagccgagaagggcctcccaccacgaggccttgtt
ccctcatttgccagtggagggactttgggcaagtcacttaacctccccct
gcatcggaatccatgtgtgtttgaggatgagagttactggcagagcccca
agcccatgcacgtgcacagccagtgcccagtatgcagtgaggggcatggt
gcccagggccagctcagagggcggggatggctcaggcgtgcaggtggaga
gcagggcttcagccccctgggagtccccagcccctgcacagcctcttctc
actctgcagGGACCCCAACATGGGCGACTCGGCTGTGCCCACACACTGGG
AACCCTACACTACGGAAAACAGCGGCTACCTGGAGATCACCAAGAAGATG
GGCAGCAGCTCCATGAAGCGGAGCCTGAGAACCAACTTCCTGCGCTACTG
GACCCTCACCTATCTGGCGCTGCCCACAGTGACCGACCAGGAGGCCACCC
CTGTGCCCCCCACAGGGGACTCCGAGGCCACTCCCGTGCCCCCCACGGGT
GACTCCGAACCGCCCCCGTGCCGCCCACGGgtactccggggccccccccc
One repeat, no intron!
cgtgccgcccacggGTGACTCCGGGGCCCCCCCCGTGCCGCCCACGGGTG
ACTCCGGGGCCCCCCCCGTGCCGCCCACGGGTGACTCCGGGGCCCCCCCC
GTGCCGCCCACGGGTGACTCCGGGGCCCCCCCCGTGCCGCCCACGGGTGA
CTCCGGGGCCCCCCCCGTGCCGCCCACGGGTGACTCCGGGGCCCCCCCCG
TGCCGCCCACGGGTGACTCCGGCGCCCCCCCCGTGCCGCCCACGGGTGAC
GCCGGGCCCCCCCCCGTGCCGCCCACGGGTGACTCCGGCGCCCCCCCCGT
GCCGCCCACGGGTGACTCCGGGGCCCCCCCCGTGACCCCCACGGGTGACT
CCGAGACCGCCCCCGTGCCGCCCACGGGTGACTCCGGGGCCCCCCCTGTG
CCCCCCACGGGTGACTCTGAGGCTGCCCCTGTGCCCCCCACAGATGACTC
CAAGGAAGCTCAGATGCCTGCAGTCATTAGGTTTTAGCGTCCCATGAGCC
TTGGTATCAAGAGGCCACAAGAGTGGGACCCCAGGGGCTCCCCTCCCATC
TTGAGCTCTTCCTGAATAAAGCCTCATACCCCT
SEQ ID NO:2
CEL coding sequence
ATGCTCACCATGGGGCGCCTGCAACTGGTTGTGTTGGGCCTCACCTGCTG
CTGGGCAGTGGCGAGTGCCGCGAAGCTGGGCGCCGTGTACACAGAAGGTG
GGTTCGTGGAAGGCGTCAATAAGAAGCTCGGCCTCCTGGGTGACTCTGTG
GACATCTTCAAGGGCATCCCCTTCGAAGCTCCCACCAAGGCCCTGGAAAA
TCCTCAGCCACATCCTGGCTGGCAAGGGACCCTGAAGGCCAAGAACTTCA
AGAAGAGATGCCTGCAGGCCACCATCACCCAGGACAGCACCTACGGGGAT
GAAGACTGCCTGTACCTCAACATTTGGGTGCCCCAGGGCAGGAAGCAAGT
CTCCCGGGACCTGCCCGTTATGATCTGGATCTATGGAGGCGCCTTCCTCA
TGGGGTCCGGCCATGGGGCCAACTTCCTCAACAACTACCTGTATGACGGC
GAGGAGATCGCCACACGCGGAAACGTCATCGTGGTCACCTTCAACTACCG
TGTCGGCCCCCTTGGGTTCCTCAGCACTGGGGACGCCAATCTGCCAGGTA
ACTATGGCCTTCGGATCAGCACATGGCCATTGCTTGGGTGAAGAGGATAT
CGCGGCCTTCGGGGGGGGGACCCCAACAACATCACGCTCTTCGGGGAGTC
TGCTGGAGGTGCCAGCGTCTCTCTGCAGACCCTCTCCCCCTACAACAAGG
GCCTCATCCGGCGAGCCATCAGCCAGAGCGGCGTGGCCCTGAGTCCCTGG
GTCATCCAGAAAAACCCACTCTTCTGGGCCAAAAAGGTGGCTGAGAAGGT
GGGTTGCCCTGTGGGTGATGCCGCCAGGATGGCCCAGTGTCTGAAGGTTA
CTGATCCCCGAGCCCTGACGCTGGCCTATAAGGTGCCGCTGGCAGGCCTG
GAGTACCCCATGCTGCACTATGTGGGCTTCGTCCCTGTCATTGATGGAGA
CTTCATCCCCGCTGACCCGATCAACCTGTACGCCAACGCCGCCGACATCG
ACTATATAGCAGGCACCAACAACATGGACGGCCACATCTTCGCCAGCATC
GACATGCCTGCCATCAACAAGGGCAACAAGAAAGTCACGGAGGAGGACTT
CTACAAGCTGGTCAGTGAGTTCACAATCACCAAGGGGCTCAGAGGCGCCA
AGACGACCTTTGATGTCTACACCGAGTCCTGGGCCCAGGACCCATCCCAG
GAGAATAAGAAGAAGACTGTGGTGGACTTTGAGACCGATGTCCTCTTCCT
GGTGCCCACCGAGATTGCCCTAGCCCAGCACAGAGCCAATGCCAAGAGTG
CCAAGACCTACGCCTACCTGTTTTCCCATCCCTCTCGGATGCCCGTCTAC
CCCAAATGGGTGGGGGCCGACCATGCAGATGACATTCAGTACGTTTTCGG
GAAGCCCTTCGCCACCCCCACGGGCTACCGGCCCCAAGACAGGACAGTCT
CTAAGGCCATGATCGCCTACTGGACCAACTTTGCCAAAACAGGGGACCCC
AACATGGGCGACTCGGCTGTGCCCACACACTGGGAACCCTACACTACGGA
AAACAGCGGCTACCTGGAGATCACCAAGAAGATGGGCAGCAGCTCCATGA
AGCGGAGCCTGAGAACCAACTTCCTGCGCTACTGGACCCTCACCTATCTG
GCGCTGCCCACAGTGACCGACCAGGAGGCCACCCCTGTGCCCCCCACAGG
GGACTCCGAGGCCACTCCCGTGCCCCCCACGGGTGACTCCGAGACCGCCC
CCGTGCCGCCCACGGGTGACTCCGGGGCCCCCCCCGTGCCGCCCACGGGT
GACTCCGGGGCCCCCCCCGTGCCGCCCACGGGTGACTCCGGGGCCCCCCC
CGTGCCGCCCACGGGTGACTCCGGGGCCCCCCCCGTGCCGCCCACGGGTG
ACTCCGGGGCCCCCCCCGTGCCGCCCACGGGTGACTCCGGGGCCCCCCCC
GTGCCGCCCACGGGTGACTCCGGCGCCCCCCCCGTGCCGCCCACGGTGAC
GCCGGCCCCCCCCCGTGCCGCCCACGGGTGACTCCGGCGCCCCCCGCCCG
TGCCGCCCACGGGTGACTCCGGGGCCCCCCCCGTGACCCCCACGGGTGAC
TCCGAGACCGCCCCCGTGCCGCCCACGGGTGACTCCGGGGCCCCCCCTGT
GCCCCCCACGGGTGACTCTGAGGCTGCCCCTGTGCCCCCCACAGATGACT
CCAAGGAAGCTCAGATGCCTGCAGTCATTAGGTTTTAG
SEQ ID NO:3
GAGGCCACCCCGTGCCCCCCACAGGGGACTCCGAGGCCACTCCCGTGCCC
CCCACGGGTGACTCCGAGACCGCCCCCGTGCCGCCCACGGGTGACTCCGG
GGCCCCCCCCGTGCCGCCCACGGGTGACTCCGGGGCCCCCCCCGTGCCGC
CCACGGGTGACTCCGGGGCCCCCCCCGTGCCGCCCACGGGTGACTCCGGG
GCCCCCCCCGTGCCGCCCACGGGTGACTCCGGCGCCCCCCCCGTGCCGCC
CACGGGTGACGCCGGGCCCCCCCCCGTGCCGCCCACGGGTGACTCCGGCG
CCCCCCCCGTGCCGCCCACGGGTGACTCCGGGGCCCCCCCCGTGACCCCC
ACGGGTGACTCCGAGACCGCCCCCGTGCCGCCCACGGGTGACTCCGGGGC
CCCCCCTGTGCCCCCCACGGGTGACTCTGAGGCTGCCCCTGTGCCCCCCA
CAGATGACTCC
SEQ ID NO:4
GAGGCCACCCCTGTGCCCCCCACAGGGGACTCCGAGGCCACTCCCGTGCC
CCCCACGGGTGACTCCGAGACCGCCCCCGTGCCGCCCACGGGTGACTCCG
GGGCCCCCCCGTGCCGCCCACGGGTGACTCCGGGGCCCCCCCCGTGCCGC
CCACGGGTGACTCCGGGGCCCCCCCCGTGCCGCCCACGGGTGACTCCGGG
GCCCCCCCCGTGCCGCCCACGGGTGACTCCGGGGCCCCCCCCGTGCCGCC
CACGGGTGACTCCGGGGCCCCCCCCGTGCCGCCCACGGGTGACTCCGGCG
CCCCCCCCGTGCCGCCCACGGGTGACGCCGGGCCCCCCCCCGTGCCGCCC
ACGGGTGACTCCGGCGCCCCCCCCGTGCCGCCCACGGGTGACTCCGGGGC
CCCCCCCGTGACCCCCACGGGTGACTCCGAGACCGCCCCCGTGCCGCCCA
CGGGTGACTCCGGGGCCCCCCCTGTGCCCCCCACGGGTGACTCTGAGGCT
GCCCCTGTGCCCCCCACAGATGACTCC
SEQ ID NO:5
GAGGCCACCCCTGTGCCCCCCACAGGGGACTCCGAGGCCACTCCTGTGCC
CCCCACGGGTGACTCCGAGACCGCCCCCGTGCCGCCCACGGGTGACTCCG
GGGCCCCCCCCGTGCCGCCCACGGGTGACTCCGGGGCCCCCCCCGTGCCG
CCCACGGGTGACTCCGGGGCCCCCCCCGTGCCGCCCACGGGTGACTCCGG
GGCCCCCCCCGTGCCGCCCACGGGTGACTCCGGGGCCCCCCCCGTGCCGC
CCACGGGTGACTCCGGCGCCCCCCCCCGTGCCGCCCACGGGTGACTCCGG
CGCCCCCCCCGTGCCGCCCACGGGTGACTCCGGGGCCCCCCCCGTGACCC
CCACGGGTGACTCCGGGGCCCCCCCTGTGCCCCCCACGGGTGACTCTGAG
GCTGCCCCTGTGCCCCCCACAGATGACTCC
SEQ ID NO:6
GAGGCCACTCCTGTGCCCCCCACGGGTGACTCCGAGACCGCCCCCGTGCC
GCCCACGGGTGACTCCGGGGCCCCCCCCGTGCCGCCCACGGGTGACTCCG
GGGCCCCCCCCGTGCCGCCCACGGGTGACTCCGGGGCCCCCCCCGTGCCG
CCCACGGGTGACTCCGGGGCCCCCCCCGTGCCGCCCACGGGTGACTCCGG
GGCCCCCCCCGTGCCGCCCACGGGTGACTCCGGGGCCCCCCCCGTGCCGC
CCACGGGTGACTCCGGCGCCCCCCCCCGTGCCGCCCACGGGTGACTCCGG
CGCCCCCCCCGTGCCGCCCACGGGTGACTCCGGGGCCCCCCCCGTGACCC
CCACGGGTGACTCCGGGGCCCCCCCTGTGCCCCCCACGGGTGACTCTGAG
GCTGCCCCTGTGCCCCCCACAGATGACTCC
SEQ ID NO:7
GAGGCCACCCCTGTGCCCCCCACAGGGGACTCCGAGGCCACTCCTGTGCC
CCCCACGGGTGACTCCGAGACCGCCCCCGTGCCGCCCACGGGTGACTCCG
GGGCCCCCCCCGTGCCGCCCACGGGTGACTCCGGGGCCCCCCCCGTGCCG
CCCACGGGTGACTCCGGGGCCCCCCCCGTGCCGCCCACGGGTGACTCCGG
GGCCCCCCCCGTGCCGCCCACGGGTGACTCCGGGGCCCCCCCCGTGCCGC
CCACGGGTGACTCCGGCGCCCCCCCCGTGCCGCCCACGGGTGACGCCGGG
CCCCCCCCCCGTGCCGCCCACGGGTGACTCCGGCGCCCCCCCCGTGCCGC
CCACGGGTGACTCCGGGGCCCCCCCCGTGACCCCCACGGGTGACTCCGAG
ACCGCCCCCGTGCCGCCCACGGGTGACTCCGGGGCCCCCCCTGTGCCCCC
CACGGGTGACTCTGAGGCTGCCCCTGTGCCCCCCACAGATGACTCC
SEQ ID NO:8
GAGGCCACCCCTGTGCCCCCCACAGGGGACTCCGAGGCCACTCCTGTGCC
CCCCACGGGTGACTCCGAGACCGCCCCCGTGCCGCCCACGGGTGACTCCG
GGGCCCCCCCCGTGCCGCCCACGGGTGACTCCGGGGCCCCCCCCGTGCCG
CCCACGGGTGACTCCGGGGCCCCCCCCGTGCCGCCCACGGGTGACTCCGG
GGCCCCCCCCGTGCCGCCCACGGGTGACTCCGGGGCCCCCCCCGTGCCGC
CCACGGGTGACTCCGGGGCCCCCCCCGTGCCGCCCACGGGTGACTCCGGG
GCCCCCCCCGTGCCGCCCACGGGTGACTCCGGCGCCCCCCCCCGTGCCGC
CCACGGGTGACTCCGGCGCCCCCCCCGTGCCGCCCACGGGTGACTCCGGG
GCCCCCCCCGTGACCCCCACGGGTGACTCCGGGGCCCCCCCTGTGCCCCC
CACGGGTGACTCTGAGGCTGCCCCTGTGCCCCCCACAGATGACTCC
SEQ ID NO:9
GAGGCCACCCCTGTGCCCCCCACAGGGGACTCCGAGGCCACTCCCGTGCC
CCCCACGGGTGACTCCGAGACCGCCCCCGTGCCGCCCACGGGTGACTCCG
GGGCCCCCCCCGTGCCGCCCACGGGTGACTCCGGGGCCCCCCCCGTGCCG
CCCACGGGTGACTCCGGGGCCCCCCCCGTGCCGCCCACGGGTGACTCCGG
GGCCCCCCCCGTGCCGCCCACGGGTGACTCCGGGGCCCCCCCCGTGCCGC
CCACGGGTGACTCCGGGGCCCCCCCCGTGCCGCCCACGGGTGACTCCGGC
GCCCCCCCCGTGCCGCCCACGGGTGACGCCGGGCCCCCCCCCCGTGCCGC
CCACGGGTGACTCCGGCGCCCCCCCCGTGCCGCCCACGGGTGACTCCGGG
GCCCCCCCCGTGACCCCCACGGGTGACTCCGAGACCGCCCCCGTGCCGCC
CACGGGTGACTCCGGGGCCCCCCCTGTGCCCCCCACGGGTGACTCTGAGG
CTGCCCCTGTGCCCCCCACAGATGACTCC
SEQ ID NO:10
GAGGCCACCCCTGTGCCCCCCACAGGGGACTCCGAGGCCACTCCCGTGCC
CCCCACGGGTGACTCCGAGACCGCCCCCGTGCCGCCCACGGGTGACTCCG
GGGCCCCCCCCGTGCCGCCCACGGGTGACTCCGGGGCCCCCCCCGTGCCG
CCCACGGGTGACTCCGGGGCCCCCCCCGTGCCGCCCACGGGTGACTCCGG
GGCCCCCCCCGTGCCGCCCACGGGTGACTCCGGGGCCCCCCCCGTGCCGC
CCACGGGTGACTCCGGGGCCCCCCCCGTGCCGCCCACGGGTGACTCCGGC
GCCCCCCCCGTGCCGCCCACGGGTGACTCCGGGCCCCCCCCCGTGCCGCC
CACGGGTGACGCCGGCGCCCCCCCCCGTGCCGCCCACGGGTGACTCCGGG
GCCCCCCCCGTGACCCCCACGGGTGACTCCGAGACCGCCCCCGTGCCGCC
CACGGGTGACTCCGGGGCCCCCCCTGTGCCCCCCACGGGTGACTCTGAGG
CTGCCCCTGTGCCCCCCACAGATGACTCC
SEQ ID NO:11
GAGGCCACCCCTGTGCCCCCCACAGGGGACTCCGAGGCCACTCCCGTGCC
CCCCACGGGTGACTCCGAGACCGCCCCCGTGCCGCCCACGGGTGACTCCG
GGGCCCCCCCCGTGCCGCCCACGGGTGACTCCGGGGCCCCCCCCGTGCCG
CCCACGGGTGACTCCGGGGCCCCCCCCGTGCCGCCCACGGGTGACTCCGG
GGCCCCCCCCGTGCCGCCCACGGGTGACTCCGGGGCCCCCCCCGTGCCGC
CCACGGGTGACTCCGGGGCCCCCCCCGTGCCGCCCACGGGTGACTCCGGC
GCCCCCCCCGTGCCGCCCACGGGTGACGCCGGGCCCCCCCCCGTGCCGCC
CACGGGTGACTCCGGCGCCCCCCCCGTGCCGCCCACGGGTGACTCCGGGG
CCCCCCCCGTGACCCCCACGGGTGACTCCGAGACCGCCCCCGTGCCGCCC
ACGGGTGACTCCGGGGCCCCCCCTGTGCCCCCCACGGGTGACTCTGAGGC
TGCCCCTGTGCCCCCCACAGATGACTCC
SEQ ID NO:12
GAGGCCACCCCTGTGCCCCCCACAGGGGACTCCGAGGCCACTCCCGTGCC
CCCCACGGGTGACTCCGAGACCGCCCCCGTGCCGCCCACGGGTGACTCCG
GGGCCCCCCCCGTGCCGCCCACGGGTGACTCCGGCGCCCCCCCCGTGCCG
CCCACGGGTGACGCCGGGCCCCCCCCCGTGCCGCCCACGGGTGACGCCGG
CGCCCCCCCCGTGCCGCCCACGGGTGACTCCGGGCCCCCCCCCGTGCCGC
CCACGGGTGACGCCGGGGCCCCCCCCGTGACCCCCACGGGTGACTCCGAG
ACCGCCCCCGTGCCGCCCACGGGTGACTCCGGGGCCCCCCCTGTGCCCCC
CACGGGTGACTCTGAGGCTGCCCCTGTGCCCCCCACAGATGACTCC
SEQ ID NO:13
EATPCPPQGTPRPLPCPPRVTPRPPPCRPRVTPGPPPCRPRVTPGPPPCR
PRVTPGPPPCRPRVTPGPPPCRPRVTPAPPPCRPRVTPGPPPCRPRVTPA
PPPCRPRVTPGPPP
SEQ ID NO:14
EATPVPPTGDSEATPVPPTGDSETAPVPPTGDSGAPPCRPRVTPGPPPCR
PRVTPGPPPCRPRVTPGPPPCRPRVTPGPPPCRPRVTPGPPPCRPRVTPA
PPPCRPRVTPGPPPCRPRVTPAPPPCRPRVTPGPPP
SEQ ID NO:15
EATPVPPTGDSEATPVPPTGDSETAPVPPTGDSGAPPVPPTGDSGAPPVP
PTGDSGAPPVPPTGDSGAPPVPPTGDSGAPPVPPTGDSGAPPRAAHG
SEQ ID NO:16
EATPVPPTGDSEATPVPPTGDSETAPVPPTGDSGAPPVPPTGDSGAPPVP
PTGDSGAPPVPPTGDSGAPPVPPTGDSGAPPVPPTGDSGAPPVPPTGDSG
APPRAAHG
SEQ ID NO:17
EATPVPPTGDSEATPVPPTGDSETAPVPPTGDSGAPPVPPTGDSGAPPVP
PTGDSGAPPVPPTGDSGAPPVPPTGDSGAPPVPPTGDSGAPPVPPTGDAG
PPPRAAHG
SEQ ID NO:18
EATPVPPTGDSEATPVPPTGDSETAPVPPTGDSGAPPVPPTGDSGAPPVP
PTGDSGAPPVPPTGDSGAPPVPPTGDSGAPPVPPTGDSGAPPvPPTGDSG
APPVPPTGDAGPPPRAAHG
SEQ ID NO:19
EATPVPPTGDSEATPVPPTGDSETAPVPPTGDSGAPPVPPTGDSGAPPVP
PTGDSGAPPVPPTGDSGAPPVPPTGDSGAPPVPPTGDSGAPPVPPTGDSG
APPVPPTGDSGAPPRAAHG
SEQ ID NO:20
EATPVPPTGDSEATPVPPTGDSETAPVPPTGDSGAPPVPPTGDSGAPPVP
PTGDSGAPPVPPTGDSGAPPVPPTGDSGAPPVPPTGDSGAPPVPPTGDSG
APPVPPTGDSGPPPVPPTGDAGAPPRAAHG
SEQ ID NO:21
EATPVPPTGDSEATPVPPTGDSETAPVPPTGDSGAPPVPPTGDSGAPPVP
PTGDSGAPPVPPTGDSGAPPVPPTGDSGAPPVPPTGDSGAPPVPPTGDSG
APPVPPTGDAGPPPVPPTGDSGAPPVPPTGDSGAPPVTPTGDSETAPVPP
TGDSGAPPVPPTGDSEAAPVPPTDDSKFAQMPAVIRF
SEQ ID NO:22
EATPVPPTGDSEATPVPPTGDSETAPVPPTGDSGAPPVPPTGDSGAPPVP
PTGDAGPPPVPPTGDAGAPPVPPTGDSGPPPVPPTGDAGAPPVTPTGDSE
TAPVPPTGDSGAPPVPPTGDSEAAPVPPTDDSKEAQMPAVIRF
SEQ ID NO:23
GTC CCT CAC TCA TTC TTC TAT GGC AAC
SEQ ID NO:24
TCC TGC AGC TTA GCC TTG GG
SEQ ID NO:25
CACACACTGGGAACCCT

The CEL genomic sequence. Sequence from http://genome.ucsc.edu. Note that in exon 11 it is indicated an intron within the VNTR matching exactly one repeat on a 17 repeat allele. Hence, the indicated intron is a result of the discrepancy between the genomic sequence from a 17 repeat allele and from the more common 16 repeat allele. Hence, there is no intron in the VNTR. Exons in upper case, everything else in lower case.

1. A method for determining a predisposition to exocrine pancreatic dysfunction in a subject comprising the step of determining in a biological sample isolated from said subject one or more SNP(s) and/or one or more DNP(s) and/or one or more genomic rearrangement(s) in a) the CEL gene and/or b) in chromosome regions comprising said CEL gene, and/or c) in transcriptional products comprising said one or more SNP(s) and/or one or more DNP(s) and/or one or more genomic rearrangement(s), and/or d) translational products arising from said transcriptional products.

2. A method for determining a predisposition to diabetes in a subject comprising the step of determining in a biological sample isolated from said subject one or more polymorphisms in a) the CEL gene and/or b) in chromosome regions comprising said CEL gene, and/or c) in transcriptional products comprising said one or more polymorphisms, and/or d) translational products arising from said transcriptional products.

3-9. (canceled)

10. The method according to any of the claims 1 or 2, wherein
i) at least one of the SNP(s) and/or DNP(s) and/or genomic rearrangement(s) is determined in a non-coding region of the CEL gene such as an intron region, or
ii) at least one of the polymorphisms is determined in a non-coding region of the CEL gene such as an intron region.

11. The method according to any of claims 1 or 2, wherein
i) at least one SNP and/or DNP and/or genomic rearrangement is determined in the region comprising a nucleotide sequence controlling expression of CEL gene, such as a promoter region, or
ii) at least one polymorphism is determined in the region comprising a nucleotide sequence controlling expression of CEL gene, such as a promoter region.

12. The method according to any of the claims 1 or 2, wherein the predisposition to exocrine pancreatic dysfunction and/or diabetes is determined by determining a polymorphism selected from the group of polymorphisms consisting of but not limited to ex11-1, ex11-2, ex11-3, ex11-4, ex11-5, ex11-6, ex11-7, ex11-8, ex11-9 and ex11-10.

13. The method according to claim 12, wherein the predisposition to exocrine pancreatic dysfunction and/or diabetes is determined by determining a polymorphism selected from the group of polymorphisms consisting of but not limited to ex11-1, ex11-2, ex11-3, ex11-4, ex11-5, ex11-6, ex11-7, ex11-8, ex11-9 and ex11-10 and at least one further polymorphism which is in linkage disequilibrium with said polymorphism.

14. The method according to claim 13, wherein at least one of the further polymorphism(s) is/are determined in the region comprising 2500 base pairs upstream or downstream from a polymorphism selected from the group of polymorphisms consisting of but not limited to ex11-1, ex11-2, ex11-3, ex11-4, ex11-5, ex11-6, ex11-7, ex11-8, ex11-9 and ex11-10.

15. (canceled)

16. The method according to any of the claims 1 or 2, wherein the polymorphism(s) is(are) present in i) a nucleotide sequence selected from SEQ ID NO: 1 ii) a nucleotide sequence having at least 90% sequence identity with a sequence of (i), or a fragment thereof, or iii) a nucleotide sequence being complementary to any of the sequences of (i) or (ii).

17-30. (canceled)

31. The method according to any of claims 1 or 2, wherein the presence or absence of a SNP or a polymorphism is determined in a target nucleic acid sequence isolated from a biological sample.

32. The method according to claim 31, said method comprising amplification of the target nucleotide sequence.

33. The method according to claim 31, wherein the nucleotide sequence is a genomic DNA sequence, an mRNA sequence, or a cDNA sequence.

34. The method according to claim 33, wherein the nucleic sequence is selected from a group of nucleic acid sequences consisting of SEQ ID NO: 1 and SEQ ID NO: 2, or a sequence complementary thereof.

35. The method according to claim 34, wherein amplification comprises use of a primer pair selected from oligonucleotide sequences being 100% identical to a subsequence of the CEL gene or a sequence being complementary thereof, comprising or adjacent to a SNP and/or DNP or other polymorphisms and/or genomic rearrangements of the invention, said SNP and/or DNP and/or genomic rearrangement being associated to exocrine pancreatic dysfunction.

36. The method according to claim 34, wherein amplification comprises use of a primer pair selected from oligonucleotide sequences being 100% identical to a subsequence of the CEL gene or a sequence being complementary thereof, comprising or adjacent to a polymorphism of the invention, said polymorphism being associated to diabetes.

37-39. (canceled)

40. The method according to any of claims 1 or 2, wherein the method employs analysis of insertion or deletion variation in the first six repeats of the CEL VNTR in exon 11 and/or determination of the number of CEL VNTR repeats.

41. The method of claim 40 employing for said analysis and/or determination duplex PCR using fluorescent primers.

42. The method of claim 41, wherein primers employed for duplex PCR are labelled with a label selected from the group consisting of FAM-blue and NED-green.

43. The method of claim 41, wherein the PCR step is followed by fragment analysis.

44. The method of claim 43, wherein insertions or deletions are detected by a shift in the migration length of a given fragment.

45. The method of claim 43, wherein two narrow peaks indicate heterozygosity for 1 bp insertion or deletion.

46-84. (canceled)


SEQUENCE LISTING (25 sequences)

SEQ ID NO: 1 - DNA, 9883 bp, Homo sapiens
ggccacccagaggctgatgctcaccatggggcgcctgcaactggttgtgttgggcctcacctgctgctgggcagtggcgagtgccgcgaaggtaagagcccagcagaggggcaggtcctgctgctctctcgctcaatcagatctggaaacttcgggccaggctgagaaagagcccagcacagccccgcagcagatcccgggcactcacgctcatttctatggggacaggtgccaggtagaacacaggatgcccaattccatttgaatttcagataaactgccaagaactgctgtgtaagtatgtcccatgcaatatttgaaacaaatttctatgggccgggcgcagtggctcacacctgcaatcccaccagtttgggaggccgaggtgggtggatcacttgaggtcaggagttggagaccagcctggccaacatggtgaaaccccgtctctactaaaaatacaaatattaatcgggcgtggtggtgggtgcctgtaatcccagctactcgggaggctgaggcaggagaaccgcttgaagctgggaggtggagattgcggtgagctgagatcacgctactgcactccagcctgggtgacagggcgagactctgtctcaaaaaatagaaaaagaaaaaaatgaaacatactaaaaaacaattcactgtttacctgaaattcaaatgtaactgggcctcttgaatttacatttgctaatcctggtgattccacctctctgttgttcccattttacagaaggggaaacgggcccaggggcagggagtgtggagagcaggcagacgggtggagagaagcaggcaggcagtttgcccagcatggcacagctgctgcctcctattcctgtgcaggaagctgaaagccgagctactccacacccgggtccgggtccctccagaaagagagccggcaggcaggagctctctcgaggcatccataaattctaccctctctgcctgtgaaggagaagccacagaaaccccaagccccacaggaagccggtgtcggtgcccggcccagtccctgcccccagcaggagtcacacaggggaccccagatcccaaccacgctgttctgccgcctgcggtgtctcaggccctggggactcctgtctccacctctgctgcctgctctccacactccctggccctgggaccgggaggtttgggcagtggtcctgggctcctgactcaaaggagaggtcaccttcttcttgggcgagctcttcttggggtgctgagaggccttcggcaggtcatcacgacccctccccatttccccaccctgaggccctctggccagtctcaattgcacagggatcacgccactggcacaaggagacacagatgcctcgcaggggatgcccacgatgcctgcatgtgttgcttctggttcctttcctccagttccaaccgccgcactctcccacaccagtgtgacagggggcccatcaccctagacttcagagggctgctgggaccctggctgggcctgggggtgtagggccaccctgcccttccccacctggaacctggcacaggtgacagccagcaagcaatgacctggtcccaccatgcaccacgggaagagggagctgctgcccaagatggacaggaggtggcactggggcagacagctgcttctcaacagggtgacttcaagcccaaaagctgcccagcctcagttccgtcagggacagagggtggatgagcaccaacctccaggcccctcgtgggggtggacagcttggtgcacagaggccattttcatggcacagggaagcgtggcgggggtgggaggtgtggtccctagggggttctttaccagcagggggctcaggaactgtggggacttgggcatggggccatcgactttgtgcccagccagctaggccctgtgcagggagatgggaggagggaaaagcaggccccacccctcagaaaggaggaaggttggtgtgaaacatcccgggtacactgagcattgggtacactcctcccgggagctggacaggcctcccatgtgatggcaaacaggccgacaggagacacggctgttgctcgtcttccacatggggaaactgaggatcggagtcaaagctgggcggccatagccagaacccaaacctccatcccacctcttggccggcttccctagtgggaacactggttgaaccagtttcctctaagattctgggagcaggacacccccagggataaggagaggaacaggaatcctaaagccctgagcattgcagggcagggggtgctgcctgggtctcctgtgcagagctgtcctgctttgaagctgtctttgcctctgggcacgcggagtcggcttgccttgccccctccggattcaggccgatggggcttgagcccccctgaccctgcccgtgtctccctcgcagctgggcgccgtgtacacagaaggtgggttcgtggaaggcgtcaataagaagctcggcctcctgggtgactctgtggacatcttcaagggcatccccttcgcagctcccaccaaggccctggaaaatcctcagccacatcctggctggcaaggtgggagtgggtggtgccggactggccctgcggcggggcgggtgagggcggctgccttcctcatgccaactcctgccacctgcagggaccctgaaggccaagaacttcaagaagagatgcctgcaggccaccatcacccaggacagcacctacggggatgaagactgcctgtacctcaacatttgggtgccccagggcaggaagcaaggtctgcctcccctctactccccaagggaccctcccatgcagccactgccccgggtctactcctggcttgagtctgggggctgcaaagctgaacttccatgaaatcccacagaggcggggaggggagcgcccactgccgttgcccagcctggggcagggcagcgccttggagcacctccctgtcttggccccaggcacctgctgcacagggacaggggaccggctggagacagggccaggcggggcgtctggggtcaccagccgctcccccatctcagtctcccgggacctgcccgttatgatctggatctatggaggcgccttcctcatggggtccggccatggggccaacttcctcaacaactacctgtatgacggcgaggagatcgccacacgcggaaacgtcatcgtggtcaccttcaactaccgtgtcggcccccttgggttcctcagcactggggacgccaatctgccaggtgcgtgggtgccttcggccctgaggtggggcgaccagcatgctgagcccagcagggagattttcctcagcacccctcaccccaaacaaccagtggcggttcacagaaagacccggaagctggagtagaatcatgagatgcaggaggcccttggtagctgtagtaaaataaaagatgctgcagaggccgggagagatggctcacgcctgtaatcccagcactttaggaggcccacacaggtgggtcacttgagcgcagaagttcaagaccagcctgaaaatcactgggagacccccatctctacacaaaaattaaaaattagctggggactgggcgcggcggctcacccctgtaatcccagcacgttgggagcccaaggtgggtagatcacctgaggtcaggagtttgagaccagcctgactaaaatggagaaacctcttctctactaaaaatacaaaattagccaggcgtggtggcgcttgcctgtaatcccagctactcgggaggctgaggcaggagaatcgcttgaactcaggaggcggaggttgcggtgagccgagatcatgccactgcactccagcctggagaacaagagtaaaactctgtctcaaaaaaaaaaaaaaaaaaaaaaatagccaggcgtggtatctcatgcctctgtcctcagctacctgggaggcagaggtggaaggatcgcttgagcccaggggttcaaagctgcagtgagccgtggtcgtgccactgcactccagcctgggcgacagagtgaggccccatctcaaaaataagaggctgtgggacagacagacaggcagacaggctgaggctcagagagaaaccaggagagcagagctgagtgagagacagagaacaataccttgaggcagagacagctgtggacacagaagtggcaggacacagacaggagggactggggcaggggcaggagaggtgcatgggcctgaccatcctgcccccgacaaacaccaccccctccagcaccacaccaacccaacctcctggggacccaccccatacagcaccgcacccgactcagcctcctggggacccacccactccagcaaccaacgtgacctagtctcctggggacccaccccctccagcaccctacccgacccagcttcttagggacccaccatttgccaactggggctctgccatggccccaactctgttgagggcatttccaccccacctatgctgatctcccctcctggaggccaggcctgggccactggtctctagcaccccctcccctgccctgcccccaggtaactatggccttcgggatcagcacatggccattgcttgggtgaagaggaatatcgcggccttcgggggggaccccaacaacatcacgctcttcggggagtctgctggaggtgccagcgtctctctgcaggtctcgggatccctgtggggagggcctgccccacaggttgagaggaagctcaaacgggaaggggagggtgggaggaggagcgtggagctggggctgtggtgctggggtgtccttgtcccagcgtggggtgggcagagtggggagcggccttggtgacgggatttctgggtcccgtagaccctctccccctacaacaagggcctcatccggcgagccatcagccagagcggcgtggccctgagtccctgggtcatccagaaaaacccactcttctgggccaaaaaggtaaacggaggagggcagggctgggcggggtgggggctgtccacatttccgttctttatcctggaccccatccttgccttcaaatggttctgagccctgagctccggcctcacctacctgctggccttggttctgcccccaggtggctgagaaggtgggttgccctgtgggtgatgccgccaggatggcccagtgtctgaaggttactgatccccgagccctgacgctggcctataaggtgccgctggcaggcctggagtgtgagtagctgctcgggttggcccatggggtctcgaggtgggggttgaggggggtactgccagggagtactccggaggagagaggaaggtgccagagctgcggtcttgtcctgtcaccaactagctggtgtctcccctcgaaggccccagctgtaagggagagggggtgccgtttcttctttttttttgagatggagtctcactgttgcccaggctggagtgcagtgtcacgatctcagctcactgcaacctccacctcctgggttcaagtgattctctgactcaacctcccatgtagctgggactacaggcacatgccaccatgcccagataatttttctgtgtgtttagtagggatggagtttcatcgtgttagctaggatgatctcggtcttgggacctcatgatctgcccacctcggcctcccaaagtgctggaattacaggcgtgagccactgtgcccggccccttctttattcttatctcccatgagttacagactcccctttgagaagctgatgaacatttggggccccctcccccacctcatgcattcatatgcagtcatttgcatataattttagggagactcatagacctcagaccaagagcctttgtgctagatgaccgttcattcattcgttcattcattcagcaaacatttactgaaccgtagcactggggcccagcctccagctccactattctgtaccccgggaaggcctggggacccattccacaaacacctctgcatgtcagccttaccagcttgctacgctaaggctgtccctcactcattcttctatggcaacatgccatgaagccaagtcatctgcacgtttacctgacatgagctcaactgcacgggctggacaagcccaaacaaagcaacccccacggccccgctagaagcaaaacctgctgtgctgggcccagtgacagccaggccccgcctgcctcagcagccactgggtcctctaggggcccgtccaggggtctggagtacaatgcagacctcccaccatttttggctgatggactggaacccagccctgagagagggagctccttctccatcagttccctcagtggcttctaagtttcctccttcctgcttcaggcccagcaaagagagagaggagagggaggggctgccgctgaagaggacagatctggccctagacagtgactctcagcctggggacgtgtggcagggcctggagacatctgtgattgtcacagctggggagggggtgctcctggcacctcgtgggtcgaggccggggatgctctaaacatcctacagggcacaggatgcccctgatggtgcagaatcaaccctgccccaagtgtccatagatcagagaagggaggacatagccaattccagccctgagaggcaaggggcggctcaggggaaactgggaggtacaagaacctgctaacctgctggctctcccacccagaccccatgctgcactatgtgggcttcgtccctgtcattgatggagacttcatccccgctgacccgatcaacctgtacgccaacgccgccgacatcgactatatagcaggcaccaacaacatggacggccacatcttcgccagcatcgacatgcctgccatcaacaagggcaacaagaaagtcacggagtaagcagggggcacaggactcaggggcgacccgtgcgggagggccgccgggaaagcactggcgagggggccagcctggaggaggaaggcattgagtggaggactgggagtgaggaagttagcaccggtcggggtgagtatgcacacaccttcctgttggcacaggctgagtgtcagtgcctacttgattcccccagggaggacttctacaagctggtcagtgagttcacaatcaccaaggggctcagaggcgccaagacgacctttgatgtctacaccgagtcctgggcccaggacccatcccaggagaataagaagaagactgtggtggactttgagaccgatgtcctcttcctggtgcccaccgagattgccctagcccagcacagagccaatgccaagtgaggatctgggcagcgggtggctcctgggggccttcctggggtgctgcaccttccagccgaggcctcgctgtgggtggctctcaggtgtctgggttgtctgggaaagtggtgcttgagtccccacctgtgcctgcctgatccactttgctgaggcctggcaagacttgagggcctctttttacctcccagcctacagggctttacaaaccctatgatcctctgccctgctcagccctgcaccccatggtccttcccactggagagttcttgagctaccttccatcccccatgctgtgtgcactgagagaacactggacaatagtttctatccactgactcttatgggcctcaactttgcccataatttcagcccaccaccacattaaaaatcttcatgtaataatagccaattataataaaaaataaggccagacacagtagctcatgcctgtaatcccagcacattgggaggtcaaggtgggaggatcacttgaggtcaggagtctgagactagtctggccaacatggcaaaaccccatctctactaaaaatacaaaaattatccaggcatggtggtgcatgcctataatcctagctactcaggaggctgaggtagcagaattgattgacccagggaggtggaggttgcagtgagccgagattacgccactgcactccagcaggggcaacagagtgagactgtgtctcgaataaataagtaaataaataataaaaataaaaaataagttaggaatacgaaaaagataggaagataaaagtatacctagaagtctaggatgaaagctttgcagcaactaagcagtacatttagctgtgagcctcctttcagtcaaggcaaaaagggaaacagttgagggcctataccttgtccaatctaattgaagaatgcacattcacttggagagcaaaatatttcttgatactgaattctagaaggaaggtgcctcacaatgttttgtggaggtgaagtataaattcagctgaaattgtggaacccatgaatccatgaatttggttctcagctttcccttccctgggtgtaagaagccccatctcttcatgtgaattccccagacacttccctgcccactgcccgggacctccctccaagtccggtctctgggctgatcggtccccagtgagcaccctgcctacttgggtggtctctcccctccaggagtgccaagacctacgcctacctgttttcccatccctctcggatgcccgtctaccccaaatgggtgggggccgaccatgcagatgacattcagtacgttttcgggaagcccttcgccacccccacgggctaccggccccaagacaggacagtctctaaggccatgatcgcctactggaccaactttgccaaaacagggtaagacgtgggttgagtgcagggcggagggccacagccgagaagggcctcccaccacgaggccttgttccctcatttgccagtggagggactttgggcaagtcacttaacctccccctgcatcggaatccatgtgtgtttgaggatgagagttactggcagagccccaagcccatgcacgtgcacagccagtgcccagtatgcagtgaggggcatggtgcccagggccagctcagagggcggggatggctcaggcgtgcaggtggagagcagggcttcagccccctgggagtccccagcccctgcacagcctcttctcactctgcagggaccccaacatgggcgactcggctgtgcccacacactgggaaccctacactacggaaaacagcggctacctggagatcaccaagaagatgggcagcagctccatgaagcggagcctgagaaccaacttcctgcgctactggaccctcacctatctggcgctgcccacagtgaccgaccaggaggccacccctgtgccccccacaggggactccgaggccactcccgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgacgccgggcccccccccgtgccgcccacgggtgactccggcgccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactccaaggaagctcagatgcctgcagtcattaggttttagcgtcccatgagccttggtatcaagaggccacaagagtgggaccccaggggctcccctcccatcttgagctcttcctgaataaagcctcatacccct

SEQ ID NO: 2 - DNA, 2238 bp, Homo sapiens
atgctcaccatggggcgcctgcaactggttgtgttgggcctcacctgctgctgggcagtggcgagtgccgcgaagctgggcgccgtgtacacagaaggtgggttcgtggaaggcgtcaataagaagctcggcctcctgggtgactctgtggacatcttcaagggcatccccttcgcagctcccaccaaggccctggaaaatcctcagccacatcctggctggcaagggaccctgaaggccaagaacttcaagaagagatgcctgcaggccaccatcacccaggacagcacctacggggatgaagactgcctgtacctcaacatttgggtgccccagggcaggaagcaagtctcccgggacctgcccgttatgatctggatctatggaggcgccttcctcatggggtccggccatggggccaacttcctcaacaactacctgtatgacggcgaggagatcgccacacgcggaaacgtcatcgtggtcaccttcaactaccgtgtcggcccccttgggttcctcagcactggggacgccaatctgccaggtaactatggccttcgggatcagcacatggccattgcttgggtgaagaggaatatcgcggccttcgggggggaccccaacaacatcacgctcttcggggagtctgctggaggtgccagcgtctctctgcagaccctctccccctacaacaagggcctcatccggcgagccatcagccagagcggcgtggccctgagtccctgggtcatccagaaaaacccactcttctgggccaaaaaggtggctgagaaggtgggttgccctgtgggtgatgccgccaggatggcccagtgtctgaaggttactgatccccgagccctgacgctggcctataaggtgccgctggcaggcctggagtaccccatgctgcactatgtgggcttcgtccctgtcattgatggagacttcatccccgctgacccgatcaacctgtacgccaacgccgccgacatcgactatatagcaggcaccaacaacatggacggccacatcttcgccagcatcgacatgcctgccatcaacaagggcaacaagaaagtcacggaggaggacttctacaagctggtcagtgagttcacaatcaccaaggggctcagaggcgccaagacgacctttgatgtctacaccgagtcctgggcccaggacccatcccaggagaataagaagaagactgtggtggactttgagaccgatgtcctcttcctggtgcccaccgagattgccctagcccagcacagagccaatgccaagagtgccaagacctacgcctacctgttttcccatccctctcggatgcccgtctaccccaaatgggtgggggccgaccatgcagatgacattcagtacgttttcgggaagcccttcgccacccccacgggctaccggccccaagacaggacagtctctaaggccatgatcgcctactggaccaactttgccaaaacaggggaccccaacatgggcgactcggctgtgcccacacactgggaaccctacactacggaaaacagcggctacctggagatcaccaagaagatgggcagcagctccatgaagcggagcctgagaaccaacttcctgcgctactggaccctcacctatctggcgctgcccacagtgaccgaccaggaggccacccctgtgccccccacaggggactccgaggccactcccgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgacgccgggcccccccccgtgccgcccacgggtgactccggcgccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactccaaggaagctcagatgcctgcagtcattaggttttag

SEQ ID NO: 3 - DNA, 461 bp, Homo sapiens
gaggccaccccgtgccccccacaggggactccgaggccactcccgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgacgccgggcccccccccgtgccgcccacgggtgactccggcgccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactcc

SEQ ID NO: 4 - DNA, 527 bp, Homo sapiens
gaggccacccctgtgccccccacaggggactccgaggccactcccgtgccccccacgggtgactccgagaccgcccccgtgccgcccacgggtgactccggggcccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgacgccgggcccccccccgtgccgcccacgggtgactccggcgccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactcc

SEQ ID NO: 5 - DNA, 430 bp, Homo sapiens
gaggccacccctgtgccccccacaggggactccgaggccactcctgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgcccccccccgtgccgcccacgggtgactccggcgccccccccgtgccgcccacgggtgactccggggccccccccgtgacccccacgggtgactccggggccccccctgtgccccccacgggtgactctgaggctgcccctgtgccccccacagatgactcc

SEQ ID NO: 6 - DNA, 430 bp, Homo sapiens
gaggccactcctgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgcccccccccgtgccgcccacgggtgactccggcgccccccccgtgccgcccacgggtgactccggggccccccccgtgacccccacgggtgactccggggccccccctgtgccccccacgggtgactctgaggctgcccctgtgccccccacagatgactcc

SEQ ID NO: 7 - DNA, 496 bp, Homo sapiens
gaggccacccctgtgccccccacaggggactccgaggccactcctgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgacgccgggccccccccccgtgccgcccacgggtgactccggcgccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactcc

SEQ ID NO: 8 - DNA, 496 bp, Homo sapiens
gaggccacccctgtgccccccacaggggactccgaggccactcctgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgcccccccccgtgccgcccacgggtgactccggcgccccccccgtgccgcccacgggtgactccggggccccccccgtgacccccacgggtgactccggggccccccctgtgccccccacgggtgactctgaggctgcccctgtgccccccacagatgactcc

SEQ ID NO: 9 - DNA, 529 bp, Homo sapiens
gaggccacccctgtgccccccacaggggactccgaggccactcccgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgacgccgggccccccccccgtgccgcccacgggtgactccggcgccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactcc

SEQ ID NO: 10 - DNA, 529 bp, Homo sapiens
gaggccacccctgtgccccccacaggggactccgaggccactcccgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgactccgggcccccccccgtgccgcccacgggtgacgccggcgcccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactcc

SEQ ID NO: 11 - DNA, 528 bp, Homo sapiens
gaggccacccctgtgccccccacaggggactccgaggccactcccgtgccccccacgggtgactccgagaccgcccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggggccccccccgtgccgcccacgggtgactccggcgccccccccgtgccgcccacgggtgacgccgggcccccccccgtgccgcccacgggtgactccggcgccccccccgtgccgcccacgggtgactccggggccccccccgtgacccccacgggtgactccgagaccgcccccgtgccgcccacgggtgactccggggccccccctgtgccccccacgggtgactctgaggctgcccctgtgccccccacagatgactcc

SEQ ID NO: 12 - DNA, 396 bp, Homo sapiens
gaggccacccctgtgccccccacaggggactccgaggccactcccgtgccccccacgggtgactccgagaccgcccccgtgccgcccacgggtgactccggggccccccccgtgccgcccacgggtgactccggcgccccccccgtgccgcccacgggtgacgccgggcccccccccgtgccgcccacgggtgacgccggcgccccccccgtgccgcccacgggtgactccgggcccccccccgtgccgcccacgggtgacgccggggccccccccgtgacccccacgggtgactccgagaccgcccccgtgccgcccacgggtgactccggggccccccctgtgccccccacgggtgactctgaggctgcccctgtgccccccacagatgactcc

SEQ ID NO: 13 - protein, 114 aa, Homo sapiens
EATPCPPQGTPRPLPCPPRVTPRPPPCRPRVTPGPPPCRPRVTPGPPPCRPRVTPGPPPCRPRVTPGPPPCRPRVTPAPPPCRPRVTPGPPPCRPRVTPAPPPCRPRVTPGPPP

SEQ ID NO: 14 - protein, 136 aa, Homo sapiens
EATPVPPTGDSEATPVPPTGDSETAPVPPTGDSGAPPCRPRVTPGPPPCRPRVTPGPPPCRPRVTPGPPPCRPRVTPGPPPCRPRVTPGPPPCRPRVTPAPPPCRPRVTPGPPPCRPRVTPAPPPCRPRVTPGPPP

SEQ ID NO: 15 - protein, 97 aa, Homo sapiens
EATPVPPTGDSEATPVPPTGDSETAPVPPTGDSGAPPVPPTGDSGAPPVPPTGDSGAPPVPPTGDSGAPPVPPTGDSGAPPVPPTGDSGAPPRAAHG

SEQ ID NO: 16 - protein, 108 aa, Homo sapiens
EATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPRAAHG

SEQ ID NO: 17 - protein, 108 aa, Homo sapiens
EATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDAGPPPRAAHG

SEQ ID NO: 18 - protein, 119 aa, Homo sapiens
EATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDAGPPPRAAHG

SEQ ID NO: 19 - protein, 119 aa, Homo sapiens
EATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDSGAPPRAAHG

SEQ ID NO: 20 - protein, 130 aa, Homo sapiens
EATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDSGPPPVPPTGDAGAPPRAAHG

SEQ ID NO: 21 - protein, 187 aa, Homo sapiens
EATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDAGPPPVPPTGDSGAPPVPPTGDSGAPPVTPTGDSETAPVPPTGDSGAPPVPPTGDSEAAPVPPTDDSKEAQMPAVIRF

SEQ ID NO: 22 - protein, 143 aa, Homo sapiens
EATPVPPTGDSEATPVPPTGDSETAPVPPTGDSGAPPVPPTGDSGAPPVPPTGDAGPPPVPPTGDAGAPPVPPTGDSGPPPVPPTGDAGAPPVTPTGDSETAPVPPTGDSGAPPVPPTGDSEAAPVPPTDDSKEAQMPAVIRF

SEQ ID NO: 23 - DNA, 27 bp, Homo sapiens
gtccctcactcattcttctatggcaac

SEQ ID NO: 24 - DNA, 20 bp, Homo sapiens
tcctgcagcttagccttggg

SEQ ID NO: 25 - DNA, 17 bp, Homo sapiens
cacacactgggaaccct